(19) United States
(12) Patent Application Publication
(10) Pub. No.: US 2020/0024339 A1
(43) Pub. Date: Jan. 23, 2020

(54) COMPOSITIONS AND METHODS FOR MODULATING CELL SIGNALING

(71) Applicants: Scholar Rock, Inc., Cambridge, MA (US); Children's Medical Center Corporation, Boston, MA (US)

(72) Inventors: Timothy Alan Springer, Newton, MA (US); Leonard Ira Zon, Wellesley, MA (US); Nagesh K. Mahanthappa, Cambridge, MA (US)

(21) Appl. No.: 16/583,799

(22) Filed: Sep. 26, 2019

Related U.S. Application Data
(63) Continuation of application No. 14/439,284, filed on Apr. 29, 2015.
(60) Provisional application No. 61/722,969, filed on Nov. 6, 2012.
(60) Provisional application No. 61/722,919, filed on Nov. 6, 2012.

(51) Int. Cl. C07K 16/22; C07K 14/495; C07K 14/475; A61K 39/395

(52) U.S. Cl. CPC C07K 16/22; C07K 14/495; C07K 2317/75; A61K 39/395; C07K 2317/76; C07K 14/475

(57) ABSTRACT

The present invention provides growth factor-directed agents (GDAs), which act as either antagonists or agonists of cell signaling, particularly in the TGF-beta and related extracellular matrix signaling pathways. Such GDAs include monoclonal antibodies, fusion proteins and novel polypeptide compositions and/or conjugates of these compositions.

### CROSS REFERENCE TO RELATED APPLICATIONS

This application claims priority to U.S. Provisional Patent Application No. 61/722,919 filed Nov. 6, 2012, entitled Compositions and Methods for Modulating Cell Signaling and U.S. Provisional Patent Application No. 61/722,969 filed Nov. 6, 2012, entitled Compositions and Methods for Modulating Cell Signaling, the contents of each of which are herein incorporated by reference in their entireties.

### SEQUENCE LISTING

The instant application contains a Sequence Listing which has been submitted electronically in ASCII format and is hereby incorporated by reference in its entirety. Said ASCII copy, created on Oct. 30, 2013, is named 20351000PCT_SL.txt and is 1,425,051 bytes in size.

### FIELD OF THE INVENTION

The present invention provides growth factor-directed agents (GDAs), which act as either antagonists or agonists of cell signaling, particularly in the TGF-beta and related extracellular matrix signaling pathways. Such GDAs may comprise antibodies, fusion proteins, polypeptides, nucleic acids and/or small molecule compositions and/or conjugates thereof.

Further provided are methods, kits and assays for exploiting the provided GDAs. Also provided are novel antigens useful, for example in the design, development, production, generation, manufacture and/or discovery of antibody based GDAs.

### BACKGROUND OF THE INVENTION

Growth factors are cell signaling molecules that stimulate a variety of cellular activities. Due to their broad-reaching influence within biological systems, growth factor signaling is tightly regulated, often through interactions with other biomolecules, the extracellular and/or cellular matrix or withing a particular cell environment or niche. These interactions may be direct or indirect.

Growth factors of the transforming growth factor beta (TGF-beta) family are involved in a variety of cellular processes. Almost all signaling in the TGF-beta family goes through a common pathway whereby a dimeric ligand is recognized by a heterotetrameric receptor complex containing two type I and two type II receptors. Each receptor has a serine-threonine kinase domain. Type II receptors phosphorylate type I receptors, which in turn phosphorylate receptor-regulated Smads that translocate to and accumulate in the nucleus and regulate transcription.

There are 33 different members of the TGF-beta family in humans. Members include the bone morphogenetic proteins (BMP), inhibin, activin, growth and differentiation factor (GDF), myostatin, nodal, anti-Mullerian hormone, and lefty proteins. Each member of the family has a prodomain of 200 to 450 residues and a C-terminal mature growth factor domain of about 110 residues (See FIG. 1).

The prodomain and mature growth factor domains are synthesized as a single polypeptide chain. The prodomains guide proper folding and dimerization of the C-terminal growth factor domains. The prodomains have very recently been recognized to have important functions in directing the growth factor (after secretion) to specific locations in the extracellular matrix and cellular matrix (ECCM), until other signals are received that cause release of the growth factor from latency. Release from latency occurs in a highly localized environment whereby most family members act over a distance of only a few cell diameters, and once they reach the circulation are rapidly cleared. Most prodomain-growth factor complexes are secreted as homodimers. However some can be secreted as heterodimers. This feature is important in the inhibin-activin family and also in BMPs, where the BMP2/BMP7 heterodimer is a potent player. Cleavage at a furin site between the prodomain and growth factor domain occurs either intracellularly prior to secretion or extracellularly after secretion. Further extracellular proteases are sometimes involved in cleavage at additional sites; these include the so-called BMP 1 or tolloid proteases.

The recent solution of the crystal structure of the latent form of TGF-beta is a first for the entire TGF-beta family and offers deep insights into these complexes (Shi, M. et al., Latent TGF-β structure and activation. Nature. 2011 Jun. 15; 474(7351):343-9).

### SUMMARY OF THE INVENTION

The present invention provides compounds, compositions, methods and kits and assays for the modulation of cell signaling, particularly the control and/or regulation of growth factor signaling.

Specifically provided are methods of modulating the function of a growth factor prodomain complex (GPC) comprising contacting the GPC with one or more growth factor-directed agents (GDAs). In this method, the GDA may comprise a monoclonal antibody. The monoclonal antibody may bind a member selected from the group consisting of a growth factor, a prodomain, the ECCM, a GPC modulatory factor or an epitope formed by the combination of a region or portion of any of the foregoing.

The present invention provides a method of stabilizing a GPC comprising contacting the GPC with a GPC targeting monoclonal antibody. In this method, stabilization may result in the inhibition of growth factor release from the GPC. Also provided is a method of increasing the level of free growth factor in a cell niche comprising contacting said GPC with a GPC targeting monoclonal antibody.

The present invention provides a method of modulating a cell signaling pathway comprising contacting a cell comprising a GPC with a GPC targeting monoclonal antibody. In one embodiment, modulation comprises upregulation or an increase in the level of a cell signaling molecule. In another embodiment, modulation comprises downregulation or a decrease in the level of a cell signaling molecule. In either of the previous two embodiments, the cell signaling molecule may be selected from the group of targets consisting of those listed in Tables 3, 4, 5, 6 and 7. In a further embodiment, the cell signaling molecule is selected from the group consisting of the TGF-beta superfamily of targets listed in Table 3.

The present invention provides a method of altering the distribution of TGF-beta polypeptides in a cell or cell niche comprising contacting a GPC of said cell or cell niche with a GDA. In one embodiment, the GDA comprises a monoclonal antibody. In a further embodiment, the TGF-beta polypeptides are selected from the group consisting of those listed in Table 3 and combinations thereof.

The present invention provides an isolated monoclonal antibody characterized in that it is specifically immunoreactive with a polypeptide having at least 10 consecutive amino acids of any of the sequences selected from the group consisting of SEQ ID NOs 1-73. In one embodiment, the isolated monoclonal antibody may be human or humanized. In another embodiment, the isolated monoclonal antibody may be immunoreactive in the extracellular environment. In another embodiment, the isolated monoclonal antibody may be immunoreactive with a GPC that has not undergone furin cleavage. In another embodiment, the isolated monoclonal antibody is an inhibitory antibody. In a further embodiment, the inhibitory antibody may inhibit the release of a growth factor from a GPC when coming into contact with the GPC. In a further embodiment, the inhibitory antibody may inhibit the signaling pathway of the growth factor when contacting a GPC. In a further embodiment, the growth factor signaling pathway may be one involving a member of the TGF-beta superfamily selected from the group consisting of any of those listed in Table 3. In another embodiment, the isolated monoclonal antibody is part of a composition. This composition may function to decrease the concentration of a growth factor in or within a cell or cell niche. This composition may further reduce the residence time of the growth factor within the cell or cell niche. The composition may elicit a neomorphic change within the cell or cell niche. The isolated monoclonal antibody may be a releasing antibody. In one embodiment, the releasing antibody promotes the release of a growth factor from a GPC when contacting the GPC. In a further embodiment, the growth factor is a TGF-beta superfamily member selected from the group consisting of any of those listed in Table 3. In another embodiment, the releasing antibody promotes a growth factor signaling pathway upon contacting a GPC. In another embodiment, contacting a GPC with the releasing antibody increases the concentration of the growth factor within a cell or cell niche. In another embodiment, contacting a GPC with the releasing antibody increases the residence time of the growth factor within the cell or cell niche. In another embodiment, contacting a GPC with the releasing antibody elicits a neomorphic change within the cell or cell niche. The composition comprising an isolated monoclonal antibody of the current invention may promote the clearance of a GPC by phagocytosis or pinocytosis.

The present invention provides a monoclonal antibody obtained by a method comprising the steps of contacting a mammal with at least one peptide, wherein the peptide is at least 70% identical to the sequences selected from the group consisting of the SEQ ID NOs 1-73, collecting cells producing the antibody from the mammal and immortalizing the cells obtained, thereby creating a hybridoma expressing the monoclonal antibodies.

The present invention provides a method for preparing a polypeptide encoding a GPC targeting antibody comprising the steps of obtaining a host cell, incubating the host cell in culture under conditions to promote expression of the polypeptide encoding a GPC targeting antibody and purifying the expressed antibody from the host cell.

The present invention provides a pharmaceutical composition comprising as an active ingredient, a monoclonal antibody specific to a GPC or component or an antibody fragment thereof comprising at least an antigen-binding portion, wherein the antibody recognizes an antigenic determinant epitope selected from the group consisting of the SEQ ID NOs 1-73 and a pharmaceutically acceptable carrier.

The present invention provides a method of treating a subject suffering from a disorder or disease associated with aberrant GPC signaling comprising the step of administering to the subject in need thereof, an antibody specific to a GPC wherein the antibody comprises an antigen-binding-portion and wherein the antibody recognizes an antigenic determinant epitope selected from the group consisting of SEQ ID NOs 1-73.

Finally, the present invention provides a kit or assay comprising a monoclonal antibody and instructions for use thereof.

### BRIEF DESCRIPTION OF THE FIGURES

FIG. 1 is a linear representation of a growth factor prodomain complex (GPC) monomer. Within the polypeptide are a secretion signal peptide, prodomain, growth factor domain as well as cysteine residue sites for disulfide bond formation. The GPC is a combination of at least 1 prodomain and 1 mature growth factor.

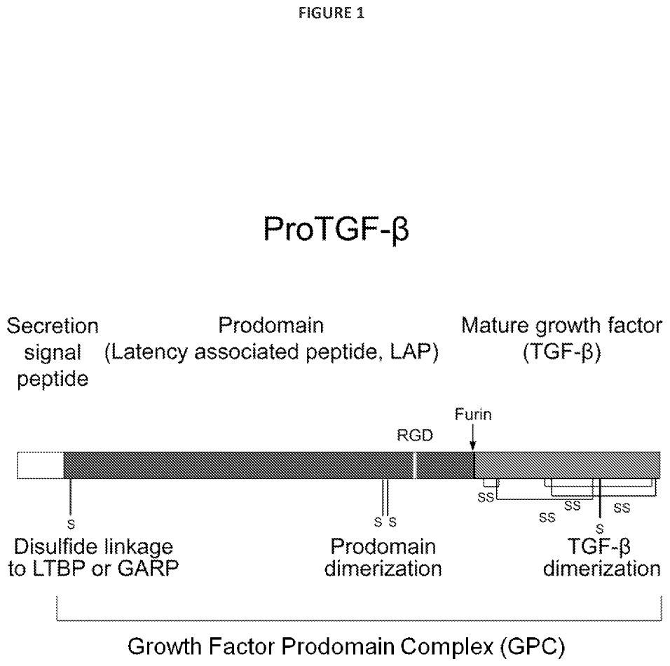

FIG. 2 is a diagram depicting alternative views of the cellular environment of a growth factor and the biomolecules that may be involved in growth factor signaling when the growth factor environment is contacted with one or more GDAs of the invention. FIG. 2A. Growth factors may comprise regulatory elements. The GDAs may interact with the regulatory element portion of the growth factor to secure the growth factor in a latent or inactive conformation or to promote growth factor release and/or activity. The ECCM is shown about the growth factor. FIG. 2B. Where the growth factor is found within a growth factor prodomain complex (GPC), a prodomain functions as a regulatory element for a growth factor. Whether the growth factor remains latent or active upon contact with a GDA may depend upon further interactions with components of the ECCM and/or GPC modulatory factors defined herein. In such embodiments, GDAs may interact with any of the elements pictured (GPC, GPC modulatory factor and/or ECCM) to stabilize the latent and/or inactive conformation of the growth factor or to promote growth factor release and/or activity.

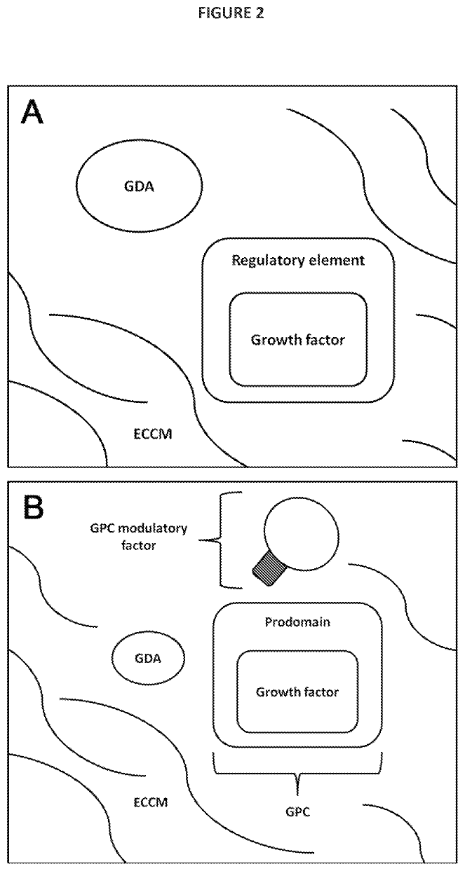

FIG. 3 is a diagram of the TGF-beta superfamily tree, where divergence is proportional to branch length.

### DETAILED DESCRIPTION

The present invention provides growth factor-directed agents (GDAs), which act as either antagonists or agonists of cell signaling, particularly in the TGF-beta and related extracellular matrix signaling pathways.

GDAs may comprise antibodies, fusion proteins, novel polypeptides, nucleic acids and/or small molecule compositions and/or conjugates thereof. Further provided are methods, kits and assays for exploiting GDAs as well as novel antigens for production of specific GDAs such as antibodies.

### I. Compositions of the Invention

### Growth Factor Directed Agents (GDAs)

In certain embodiments, the present invention provides compounds as well as compositions that comprise at least one growth factor directed agent, or GDA. As used herein, the term “growth factor” refers to one or more biomolecules that stimulate changes in cell behavior including, but not limited to changes in cell growth, cell proliferation and cell differentiation. Growth factors may be peptides or polypeptides and may be associated with other types of biomolecules. Common growth factors include, but are not limited to bone morphogenetic proteins (BMPs), brain-derived neurotrophic factor (BDNF), epidermal growth factor (EGF), erythropoietin (EPO), fibroblast growth factor (FGF), glial cell line-derived neurotrophic factor (GDNF), granulocyte colony-stimulating factor (G-CSF), granulocyte macrophage colony-stimulating factor (GM-CSF), growth differentiation factor-9 (GDF9), hepatocyte growth factor (HGF), hepatoma-derived growth factor (HDGF), insulin-like growth factor (IGF), migration-stimulating factor, nerve growth factor (NGF), placental growth factor (PGF), platelet-derived growth factor (PDGF), thrombopoietin (TPO), transforming growth factor alpha (TGF-α) family members, transforming growth factor beta (TGF-beta) family members, tumor necrosis factor-alpha (TNF-α), vascular endothelial growth factor (VEGF), and Wnt and Notch signaling pathway members.

As used herein, the term “growth factor directed agent” or “GDA” refers to an exogenously supplied compound, composition or entity which functions to alter, modulate, antagonize, agonize or in some way perturb growth factor associated cell signaling. In general, GDAs may be any chemical entity. In some embodiments, GDAs may comprise polymeric or nonpolymeric entities. In some embodiments, GDAs may comprise one or more antibodies, fusion proteins, novel polypeptides, nucleic acids, glycans, lipids and/or small molecule compositions and/or conjugates and/or combinations thereof. In some embodiments, GDAs are directed toward one or more regulatory elements. In some embodiments, GDAs modulate TGF-beta or TGF-beta family member growth factor associated cell signaling. In some embodiments, GDAs modulate growth factor associated cell signaling by contacting at least one growth factor prodomain complex (GPC). In some embodiments, GDAs modulate growth factor associated cell signaling by contacting one or more extacellular and/or cellular matrix (ECCM) components.

### Growth Factor Directed Agents (GDAs): Target Sites

In some embodiments, GDAs are directed toward at least one target site. As used herein, the term “target site” refers to one or more regions of interaction between GDA compounds or compositions and biomolecules or biostructures in a cell, tissue, organ or organism. In some embodiments, target sites may reside exclusively on one protein or may be formed by two or more proteins.

In some embodiments, target sites may comprise biomolecules including, but not limited to proteins, sugars, lipids and nucleic acid molecules. In some embodiments, target sites comprise any other form of binding epitope. GDA target sites contemplated include any and all possible regions of interaction for altering, enhancing or inhibiting growth factor function. A target site may be found in or on a growth factor, a growth factor prodomain, a growth factor prodomain complex (GPC), a GPC modulatory factor or any biomolecule of the ECCM.

Alternatively or additionally, such sites may include regions of interaction between ECCM components, regulatory elements, growth factors, receptors, ligands, GPCs and GPC modulatory factors.

As used herein, the term “GPC” or “growth factor prodomain complex” refers to a combination of mature growth factor with its prodomain. In some embodiments, polypeptides of GPC prodomains are contiguous with a growth factor. In some embodiments, polypeptides of GPC prodomains are not linked via regular peptide bonds with a growth factor, but remain associated through chemical bonds and/or molecular interactions including, but not limited to non-covalent bonds, ionic bonds, hydrogen bonds, hydrophobic interactions, dipole-dipole interactions and/or Van der Waals forces. In some embodiments, GPCs comprise TGF-beta family members.

Target sites may include one or more regulatory elements. As used herein, the term “regulatory element” refers to one or more regions, moieties, or domains, contiguous with, present within or on, and/or bound directly or indirectly to one or more growth factors that modulates growth factor activity. GDAs may bind or interact with any number of target sites on or along regulatory elements and/or GPCs or associated structures to agonize, antagonize or otherwise modulate growth factor activity.

In some embodiments, contact between a GDA and a regulatory elements leads to a conformational, structural and/or 3-dimensional change in regulatory element and/or growth factor structure. In some embodiments, contact between GDAs and regulatory elements leads to release of growth factor from regulatory elements.

In some embodiments, contact between GDAs and regulatory elements results in an increase in growth factor activity. In some embodiments, contact between GDAs and regulatory elements leads to a decrease in growth factor activity.

In some embodiments, contact between GDAs and regulatory elements prevents release of a growth factor from a regulatory element.

In some embodiments, regulatory elements comprise at least one prodomain of a GPC. As used herein, the term “prodomain” refers to an N-terminal protein domain synthesized contiguously with functional proteins, but typically cleaved from mature proteins. Prodomains may be a few (10) to hundreds of amino acids in length. In some embodiments, prodomains are post-translationally modified. Such post-translational modifications include, but are not limited to phosphorylation, ubiquitination, glycosylation and pyrolization.

In some embodiments, GDAs may serve to stabilize retention of mature growth factors by GPCs to reduce growth factor activity. GDAs may release mature growth factors from GPCs to enhance growth factor activity.

In some embodiments, GDAs modulate the ratio of active and/or free growth factor relative to inactive and/or sequestered growth factor upon introduction of GDAs to one or more GPCs or one ore more natural depots of GPCs or to any other forms of growth factor sequestration. In some embodiments, GDA-induced modulations such growth factor ratios may be localized to a particular cell niche. In some embodiments, GPC contact with a GPC modulatory factor may stimulate release of mature growth factor in the absence of a GDA.

As used herein, the term “GPC modulatory factor” refers to any endogenous biomolecule or biomolecules capable of modulating GPCs through direct or indirect interaction with the GPC. GPC modulatory factors include, but are not limited to integrins, tolloid/BMP proteases, thrombospondin, fibrillins, metalloproteases, crypto and furin/PACE. In some embodiments, GPC modulatory factors comprise one or more cells or entities bound to cells.

Administration or contact with one or more GDAs of the invention may, in turn, trigger the contact of GPCs with GPC modulatory factors, producing any of several outcomes. In some embodiments, contact of GPCs with GPC modulatory factors may result in release of mature growth factor from a GPC. In some embodiments, contact of GPCs with GPC modulatory factors may result in retention of growth factors by GPCs. In some embodiments, contact of GPCs with GPC modulatory factors may result in increased growth factor activity. In some embodiments, contact of GPCs with GPC modulatory factors may result in decreased growth factor activity.

### Growth Factor Directed Agents (GDAs): ECCM and Niches

As used herein, the term, “extracellular and cellular matrix” or ECCM refers to both the extracellular matrix and the cellular matrix as well as additional proteins or molecules (including but not limited to proteins, nucleic acids, membranes, lipids and sugars) that may be directly or indirectly associated with components of the extracellular and cell surface environments. In some embodiments, ECCM components include molecules such as, but not limited to, latent TGF-beta-binding protein (LTBP), fibrillin, elastin, collagen and the like. In some embodiments, ECCM components include cells and platelets. In some embodiments, ECCM components include cell and platelet surface associated proteins and molecules including, but not limited to glycoprotein-A repetitions predominant protein (GARP), receptors, proteoglycans, carbohydrate molecules, integral membrane proteins, glycolipids and the like. In some embodiments, ECCM components include GPC modulatory factors. In some embodiments, modulation of a growth factor signaling pathway may occur within a particular cell niche. Therefore, GDAs may be designed to operate, target and/or function within a particular cell niche. As used herein, the term “cell niche” refers to a unique set of physiologic conditions in a cellular system within a tissue, organ or organ system within or derived from a mammalian organism. A cell niche may occur in vivo, in vitro, ex vivo, or in situ. Given the complex nature and the dynamic processes involved in growth factor signaling, a cell niche may be characterized functionally, spatially or temporally or may be used to refer to any environment that encompasses one or more cells. As such, in some embodiments a cell niche includes the environment of any cell adjacent to another cell that provides support, such as for example a nurse cell.

As used herein, a “functional cell niche” is one which is characterized by a set of biomolecular functions, whether catalytic (such as those involving enzymes), structural (such as co-factors, etc), electronic (such as ion concentration or pH measures), replicative, regenerative and/or repairing (such as DNA and RNA functions), transmissive (such as ion channels, etc), functioning milieus (such as a lipid or glycosylation layers or coatings, matrices, etc), apoptotic or combinations thereof. In some embodiments, GDAs within a functional cell niche will produce one or more phenotypic changes in at least one biomolecular function associated with a functional cell niche. An example of a functional cell niche is one that involves TGF-beta growth factor signaling. GDAs acting within a TGF-beta functional cell niche, may produce phenotypic changes such as the modulation (up or down) of growth factor or GPC levels. Changes effected by the action of GDAs may also manifest as downstream changes associated with biomolecular functions triggered by growth factor or GPC levels.

As used herein, a “spatial cell niche” is one which is characterized by orientation in space. A spatial cell niche, when found within an organ or tissue, typically includes only cells within that organ or tissue. A spatial cell niche may comprise a microenvironment within a cell niche in which a GDA functions. In such cases, cell niches may be defined by contacted cells and neighboring cells in direct contact with contacted cells. Examples of spatial cell niches include sites or regions in tissues or organs, such as for example in tumors. GDAs may act within a spatial niche to effect changes in niches or overall organisms. Such changes may be phenotypic changes as evidenced by modulation (up or down) of biomolecule level and/or activity or by structural changes in the spatial niches such as cell shapes or connectivity to other cells.

As used herein, a “temporally defined cell niche” is one which is characterized by timed events. A temporal cell niche may comprise a series or set of events such as time to onset or duration of GDA effects, measures in Tₘₐₓ, Cₘₐₓ or other pharmacokinetic or pharmacodynamic parameters typically measured over time.

In some embodiments, GDAs may act to modulate one or more distinct or separate cell niches and may act repeatedly. In some embodiments, GDAs modulate the ratio of active and/or free growth factor relative to inactive and/or sequestered growth factor upon the introduction of GDAs to one or more cell niches, one or more natural depots or to any other sites of growth factor sequestration. In some embodiments, the ratio may be modulated by at least 10%, 20%, 30%, 40%, 50% or more.

Modulation of a cell niche may be by at least 10%, 20%, 30%, 40%, 50% or more. Measurement of perturbation or modulation will be determined based upon the type of cell niche and such methods are known in the art to those of skill. For example, alteration or modulation of a spatial cell niche may be defined by at least 10% change in the location or conformation of a cell or cell microenvironment. Such changes are easily detectible with standard microscopic techniques, with fluorescent microscopic techniques or by using labeling studies. Such changes may also be detected at the level of protein and/or gene expression using techniques known in the art including, but not limited to Western blot, Northern blot, reverse transcriptase (RT) polymerase chain reaction (PCR) conversion of mRNA to DNA followed by PCR amplification for use in Southern blotting or Real Time RT-PCR, PCR array, gene array, cell-based reporter assays and the like. In some embodiments, the sensitivity of such assays is at least 10%, at least 20%, at least 30% or more than 30%. In some embodiments, modulation may be measured according to methods that apply surface Plasmon resonance technology.

In some embodiments, modulation may be measured through the detection of a biological response and/or the detection of protein modifications resulting from cell signaling events such as protein modifications in cell signaling cascades. Such modifications include, but are not limited to protein phosphorylation, protein dephosphorylatoin, protein ubiquitinylation, protein degradation, protein cleavage, protein localization and protein mislocalization.

In the case of TGF-beta family member signaling, phosphorylation of SMAD proteins or activation of transcription of downstream genes may be detected.

In one embodiment, detection methods may include the use of antibodies to phosphoSMAD followed by immunoperoxidase-based visualization of bound antibodies. Alternatively, induction of gene expression may be detected in monolayer cell cultures or tissue sections using a TGF-beta-inducible promoter driving expression of GFP or luciferase. Methods disclosed above have been described in previous publications including, Wang, R. et al., GARP regulates the bioavailability and activation of TGFβ. Mol Biol Cell. 2012 March; 23(6):1129-39; Abe, M. et al., An assay for transforming growth factor-beta using cells transfected with a plasminogen activator inhibitor-1 promoter-luciferase construct. Anal Biochem. 1994 Feb. 1; 216(2):276-84; U.S. Pat. Nos. 7,015,906; 5,967,979; 7,863,569; 7,297,961; 7,738,107; 6,784,999 and 7,358,056 all of which are incorporated herein by reference in their entirety.

### Growth Factor Directed Agents: Antibodies

GDAs may comprise antibodies or fragments thereof. As used herein, the term “antibody” is used in the broadest sense and specifically covers various embodiments including, but not limited to monoclonal antibodies, polyclonal antibodies, multispecific antibodies (e.g. bispecific antibodies formed from at least two intact antibodies), and antibody fragments such as diabodies so long as they exhibit a desired biological activity. Antibodies are primarily amino-acid based molecules but may also comprise one or more modifications such as with sugar moieties.

“Antibody fragments” comprise a portion of an intact antibody, preferably comprising an antigen binding region thereof. Examples of antibody fragments include Fab, Fab′, F(ab′)₂, and Fv fragments; diabodies; linear antibodies; single-chain antibody molecules; and multispecific antibodies formed from antibody fragments. Papain digestion of antibodies produces two identical antigen-binding fragments, called “Fab” fragments, each with a single antigen-binding site. Also produced is a residual “Fc” fragment, whose name reflects its ability to crystallize readily. Pepsin treatment yields an F(ab′)₂ fragment that has two antigen-binding sites and is still capable of cross-linking antigen. GDAs may comprise one or more of these fragments. For the purposes herein, an “antibody” may comprise a heavy and light variable domain as well as an Fc region.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain.

As used herein, the term “variable domain” refers to specific antibody domains that differ extensively in sequence among antibodies and are used in the binding and specificity of each particular antibody for its particular antigen.

As used herein, the term “Fv” refers to antibody fragments which contain a complete antigen-recognition and antigen-binding site. This region consists of a dimer of one heavy chain and one light chain variable domain in tight, non-covalent association.

Antibody “light chains” from any vertebrate species can be assigned to one of two clearly distinct types, called kappa and lambda based on amino acid sequences of their constant domains. Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2.

“Single-chain Fv” or “scFv” as used herein, refers to a fusion protein of V_{H} and V_{L} antibody domains, wherein these domains are linked together into a single polypeptide chain. In some embodiments, the Fv polypeptide linker enables the scFv to form the desired structure for antigen binding.

The term “diabodies” refers to small antibody fragments with two antigen-binding sites, which fragments comprise a heavy chain variable domain V_{H} connected to a light chain variable domain V_{L} in the same polypeptide chain. By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993), the contents of each of which are incorporated herein by reference in their entirety.

The term “monoclonal antibody” as used herein refers to an antibody obtained from a population of substantially homogeneous cells (or clones), i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen

The modifier “monoclonal” indicates the character of the antibody as being obtained from a substantially homogeneous population of antibodies, and is not to be construed as requiring production of the antibody by any particular method. The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from the hypervariable region from an antibody of the recipient are replaced by residues from the hypervariable region from an antibody of a non-human species (donor antibody) such as mouse, rat, rabbit or nonhuman primate having the desired specificity, affinity, and capacity.

The term “hypervariable region” when used herein in reference to antibodies refers to regions within the antigen binding domain of an antibody comprising the amino acid residues that are responsible for antigen binding. The amino acids present within the hypervariable regions determine the structure of the complementarity determining region (CDR). As used herein, the “CDR” refers to the region of an antibody that comprises a structure that is complimentary to its target antigen or epitope.

In some embodiments, GDAs of the present invention may be antibody mimetics. The term “antibody mimetic” referes to any molecule which mimics the function or effect of an antibody and which binds specifically and with high affinity to their molecular targets. In some embodiments, antibody mimetics may be monobodies, designed to incorporate the fibronectin type III domain (Fn3) as a protein scaffold (U.S. Pat. Nos. 6,673,901; 6,348,584). In some embodiments, antibody mimetics may be those known in the art including, but are not limited to affibody molecules, affilins, affitins, anticalins, avimers, DARPins, Fynomers and Kunitz and domain peptides. In other embodiments, antibody mimetics may include one or more non-peptide region.

As used herein, the term “antibody variant” refers to a biomolecule resembling an antibody in structure and/or function comprising some differences in their amino acid sequence, composition or structure as compared to a native antibody.

The preparation of antibodies, whether monoclonal or polyclonal, is know in the art. Techniques for the production of antibodies are well known in the art and described, e.g. in Harlow and Lane “Antibodies, A Laboratory Manual”, Cold Spring Harbor Laboratory Press, 1988 and Harlow and Lane “Using Antibodies: A Laboratory Manual” Cold Spring Harbor Laboratory Press, 1999.

In one embodiment, GDAs comprising antibodies, antibody fragments, their variants or derivatives as described above are specifically immunoreactive with GPCs, GPC modulatory factors or the ECCM. In a preferred embodiment, GDAs comprising antibodies or antibody fragments are specifically immunoreactive with the TGF-beta GPC or TGF-beta growth factor. GDAs comprising antibodies or fragments of antibodies may also bind to target sites on TGF-beta family member GPCs, TGF-beta family members, Wnt signaling components or Notch signaling components.

In some embodiments, antibodies of the present invention may be immunoreactive to receptors, natural antagonists or other components of the growth factor cell signaling machinery.

### Growth Factor Directed Agents (GDAs): Antibodies, Characterization

Antibodies of the present invention may be characterized by their target molecule(s), by the antigens used to generate them, by their function (whether as agonists or antagonists) and/or by the cell niche in which they function.

GDA antibodies of the present invention may function as releasing (agonist) or inhibiting (antagonist) antibodies. As used herein, the term “releasing antibody” refers to an antibody that increases the ratio of active and/or free growth factor relative to inactive and/or sequestered growth factor upon the introduction of the antibody to a GPC, cell, niche, natural depot or any other site of growth factor sequestration. In this context, the releasing antibodies may be characterized as agonists. As used herein, a “natural depot” is a location within a cell, tissue or organ where increased levels of a biomolecule or ion are stored. For example, the extracellular matrix may act as a natural depot for one or more growth factors.

The contact necessary for release can be defined as direct or indirect contact of antibody with a GPC or a component thereof or with a cellular structure such as cell matrix or fibrillin for release of growth factor. Release of at least 5%, 10%, 20%, 30%, 40%, 50%, 60%, 70%, 80%, 90% or more of growth factor is sufficient to characterize a GDA antibody as a releasing antibody. It is understood that growth factor release after GDA antibody administration may be local and may occur over a sustained period of time and may include peaks or spikes of release. GDA antibodies of the present invention may act to release a growth factor over minutes, hours, days or longer.

Release profiles may have an initial peak or burst within from about 4 hours to about 7 days of contacting in vivo or shorter periods in vitro. For example, initial peak or burst may occur from about 4 hours to about 5 hours, or from about 4 hours to about 6 hours, or from about 4 hours to about 7 hours, or from about 4 hours to about 8 hours, or from about 4 hours to about 9 hours, or from about 4 hours to about 10 hours, or from about 4 hours to about 11 hours, or from about 4 hours to about 12 hours, or from about 4 hours to about 24 hours, or from about 4 hours to about 36 hours, or from about 4 hours to about 48 hours, or from about 1 day to about 7 days, or from about 1 day to about 2 days, or from about 1 day to about 3 days, or from about 1 day to about 4 days, or from about 4 days to about 5 days, or from about 4 days to about 6 days, or from about 4 days to about 7 days. GDAs may stimulate the release of 5 to 100% of the growth factor present. For example, the percent of growth factor release may be from about 5% to about 10%, or from about 5% to about 15%, or from about 5% to about 20%, or from about 5% to about 25%, or from about 10% to about 30%, or from about 10% to about 40%, or from about 10% to about 50%, or from about 10% to about 60%, or from about 20% to about 70%, or from about 20% to about 80%, or from about 40% to about 90%, or from about 40% to about 100%.

As used herein, the term “inhibitory antibody” or “stabilizing antibody” refers to an antibody that decreases the ratio of active and/or free growth factor relative to inactive and/or sequestered growth factor upon the introduction of the antibody to one or more GPCs, cell, niches, natural depots and/or any other sites of growth factor sequestration. In this context, antibodies may be characterized as antagonists. As used herein, an “antagonist” is one which interferes with or inhibits the physiological action of another. Antagonist action may even result in stimulation or activation of signaling downstream and hence may act agonistically relative to another pathway, separate from the one being antagonized. Pathways are interelated, so, in one nonlimiting example, a TGF-beta antagonist could act as a BMP agonist and vice versa. As used herein, “downstream” means any signaling or cellular event that happens after the action, binding or targeting by GDAs.

Contact necessary for inhibition or stabilization may be direct or indirect contact between antibody and GPC or a component thereof or with cellular structures such as cell matrix or fibrillin whereby release of growth factor is inhibited Inhibition of release of at least 5%, 10%, 20%, 30%, 40%, 50%, 60%, 70%, 80%, 90% or more of growth factors is sufficient to characterize GDA antibodies as inhibitory or stabilizing. Inhibitory GDA antibodies may stabilize GPCs and trap them as heterodimers.

It is understood that inhibition of growth factor release after contact with a GDA antibody may be local and may occur over a sustained period of time and may include peaks, troughs or spikes Inhibitory antibodies which may also function to stabilize GPCs may be defined by their release kinetics. Release of growth factor and corresponding release kinetics, even locally, may be directly measured or inferred by downstream signaling events. In some embodiments, changes in protein or nucleic acid concentrations or phenotypic responses may be indicative of the effects of GDAs.

GDA antibodies may act to inhibit release of a growth factor over minutes, hours, or days Inhibition or stabilization profiles may have an initial trough within from about 4 hours to about 7 days of introduction in vivo or shorter periods in vitro. For example, initial trough of inhibition or stabilization may occur from about 4 hours to about 5 hours, or from about 4 hours to about 6 hours, or from about 4 hours to about 7 hours, or from about 4 hours to about 8 hours, or from about 4 hours to about 9 hours, or from about 4 hours to about 10 hours, or from about 4 hours to about 11 hours, or from about 4 hours to about 12 hours, or from about 4 hours to about 24 hours, or from about 4 hours to about 36 hours, or from about 4 hours to about 48 hours, or from about 1 day to about 7 days, or from about 1 day to about 2 days, or from about 1 day to about 3 days, or from about 1 day to about 4 days, or from about 4 days to about 5 days, or from about 4 days to about 6 days, or from about 4 days to about 7 days. GDA introduction may lead to inhibition or stabilization of 5% to 100% of growth factor present. For example, the percent of growth factor inhibition or stabilization may be from about 5% to about 10%, or from about 5% to about 15%, or from about 5% to about 20%, or from about 5% to about 25%, or from about 10% to about 30%, or from about 10% to about 40%, or from about 10% to about 50%, or from about 10% to about 60%, or from about 20% to about 70%, or from about 20% to about 80%, or from about 40% to about 90%, or from about 40% to about 100%.

GDAs comprising antibodies may act to decrease local concentration of one or more GPC through removal by phagocytosis, pinocytosis, or inhibiting assembly in the ECCM. GDA introduction may lead to the removal of 5% to 100% of the growth factor present in a given area. For example, the percent of growth factor removal may be from about 5% to about 10%, or from about 5% to about 15%, or from about 5% to about 20%, or from about 5% to about 25%, or from about 10% to about 30%, or from about 10% to about 40%, or from about 10% to about 50%, or from about 10% to about 60%, or from about 20% to about 70%, or from about 20% to about 80%, or from about 40% to about 90%, or from about 40% to about 100%.

Measures of release, inhibition or removal of a growth factor may be made relative to a standard or to the natural release or activity of growth factor under normal physiologic conditions, in vitro or in vivo. Measurements may also be made relative to the presence or absence of GDA antibodies. Such methods of measuring growth factor levels, release, inhibition or removal include standard measurement in tissue or fluids such as serum or blood such as Western blot, enzyme-linked immunosorbent assay (ELISA), activity assays, reporter assays, luciferase assays, polymerase chain reaction (PCR) arrays, gene arrays, Real Time reverse transcriptase (RT) PCR and the like.

GDA antibodies may bind or interact with any number of locations on or along GPCs or their associated structures to either enhance or inhibit growth factor signaling. GDA antibody target sites contemplated include any and all possible sites for altering, enhancing or inhibiting GPC function. In some embodiments, such sites include, but are not limited to sites on or within growth factors, regulatory elements, GPCs, GPC modulatory factors, growth factor receiving cells or receptors, ECCM components and/or epitopes formed by combining regions or portions of any of the foregoing.

GDA compounds of the present invention exert their effects via binding (reversibly or irreversibly) to one or more target sites. While not wishing to be bound by theory, target sites which represent a binding site for an antibody, are most often formed by proteins or protein domains or regions. However, target sites may also include biomolecules such as sugars, lipids, nucleic acid molecules or any other form of binding epitope.

One type of antagonist antibody of the present invention would bind to the prodomain of TGF-beta and stabilize against integrin-mediated release, for example, by blocking the RGD site or by stabilizing the structure. Such an antibody would be useful in the treatment of Camurati-Engelmann disease, in which mutations in the prodomain cause excessive TGF-beta activation. Such antibodies would also be useful in Marfan's syndrome, in which mutations in fibrillins or LTBP alter TGF-beta and BMP activation.

In some embodiments, GDA antibodies selectively inhibit the release of TGF-beta from GPCs associated with LTBP's but not those associated with GARP. Such antibodies function as anti-fibrotic therapeutics but exhibit minimal inflammatory effects. In one embodiment, GPC-LTBP complex binding antibodies do not bind GPC-GARP complexes. Such antibodies, while not specific to a particular LTBP or GPC, do bind to the GPC close to or overlapping the GARP binding site, such that binding is impeded by GARP, but not by LTBPs. In one embodiment, antibodies are provided that selectively bind a combinatorial epitope between GARP and proTGF-beta. In one embodiment, GDA antibodies are provided which induce release of TGF-beta from GARP-proTGF-beta complexes. Such antibodies are selected for their ability to bind to the GARP prodomain binary complex but not GARP-proTGF-beta ternary complex, GARP alone, or prodomain alone.

Alternatively or additionally, GDA antibodies of the present invention may function as ligand mimetics which would induce internalization of the GPC. They may act as nontraditional payload carriers, acting to deliver or ferry bound or conjugated drug payloads to specific GPC or GPC-related sites.

Changes elicited by antibodies of the present invention may result in a neomorphic change in the cell. As used herein, “a neomorphic change” is a change or alteration that is new or different. For example, an antibody that elicits the release or stabilization of a growth factor not typically associated with the GPC targeted by the antibody, would be a neomorphic antibody and the release would be a neomorphic change.

In some embodiments, compounds or agents of the invention act to alter or control proteolytic events. Such events may be intracellular or extracellular. They may include the alteration of furin cleavage or other proteolytic processing events. Such events may comprise proteolytic processing of growth factor signaling molecules or downstream cascades initiated by growth factor signaling molecules.

GDA antibodies may induce or inhibit dimerization or multimerization of growth factors (ligands) or their receptors. Such action may be through the stabilization of monomeric, dimeric or multimeric forms. It may also be through the disruption of dimeric or multimeric complexes.

GDA antibodies may act on homo or heterodimers of the monomeric units comprising either receptor groups or GPCs or other signaling molecule pairs.

Antibodies of the present invention may be internalized into cells prior to binding to its target. Upon internalization, they may act to increase or decrease a signaling event, release or stabilize one or more GPCs, block or facilitate growth factor release, or alter one or more cell niche.

GDA compounds and compositions of the invention may also alter the residence time of the growth factor in the GPC or GPC in the extracellular/cellular matrix (ECCM). This alteration may result in irreversible localization or transient localization.

### Growth Factor Directed Agents: Antigens

Although it was recently realized that TGF-beta family members all have a prodomain with a common three dimensional structure, the sequence and hence structure of the prodomains are highly divergent (Shi, M. et al., Latent TGF-β structure and activation. Nature. 2011 Jun. 15; 474(7351):343-9). This divergence encodes great specialization of the prodomains in regulating targeting and release of the growth factors in the extracellular environment.

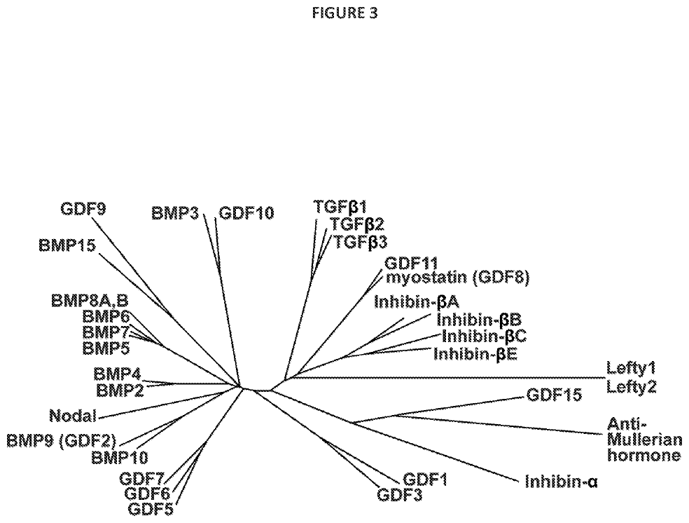

There are subfamilies within the TGF-beta family; these correspond to the major branches shown in FIG. 3, where divergence is proportional to branch length. Between human and mouse, TGF-beta1, 2 and 3 prodomains are 85, 96, and 97% identical; whereas the growth factor domains are 99, 97, and 100% identical. Thus, both the prodomains and growth factor domains are highly conserved across species. By contrast, among human TGF-beta1/TGF-beta 2, TGF-beta 1/TGF-beta 3, and TGF-beta 2/TGF-beta 3, the prodomains are 34, 33, and 47% identical; whereas the mature growth factor domains are 71, 77, and 79% identical. Thus, even within a TGF-beta subfamily, the prodomains are poorly conserved, whereas the growth factor domains, which bind to identical type I and II receptors are much better conserved. In keeping with the high conservation among the TGF-beta 1, 2, and 3 growth factor domains of 71 to 79%, it has been possible to raise antibodies which are cross-reactive, but not specific, among TGF-beta 1, 2, and 3 in humans. These antibodies have been in use in clinical trials.

The great difference among the 3 TGF-beta prodomains is reflected by diversity in the biological mechanisms that release them from latency. For example, both TGF-beta 1 and TGF-beta 3 have an RGD sequence in their prodomains that is required for their activation, which occurs as a consequence of force exerted on the prodomain by integrins αᵥβ₆ and αᵥβ₈. TGF-beta 2 has no such RGD sequence and its activation involves distinct mechanisms that include proteases.

Within the TGF-beta subfamily as within other subfamilies there are also some subtle differences. For example, while TGF-beta 1, 2 and 3 bind exactly the same type I and type II receptors, TGF-beta 2 has markedly lower affinity; thus its signaling is uniquely dependent on a co-receptor known as beta-glycan. TGF-beta 2 also differs from TGF-beta 1 and 3 by having two splice variants that differ by a short insertion in the prodomain. Heterodimers may also form among 1, 2, and 3.

The high sequence identity between human and mouse orthologs in the TGF-beta family has important implications for the derivation of antibodies. It has been quite difficult to raise antibodies specific for TGF-beta 2 and TGF-beta 3 by immunization across species, in agreement with their 96 to 97% identity cited above. Furthermore there are few antibodies to TGF-beta 1, and their biology is poorly characterized. Add to this that the divergence between human and mouse TGF-beta1 is not evenly distributed throughout the sequence but limited to certain structural loops (the β1-β2, β3-β4, and α4-β7 loops and to a region from the β8-β9 loop to the portion of the β9-β10 loop preceding the RGD sequence) and antibody development is almost impossible.

The present invention utilizes the divergent prodomain polypeptide and peptide sequences, in whole or in part, as antigens to design and develop GDAs using the methods described herein.

As used herein, an “antigen” is a substance which induces or evokes an immune response in an organism. An immune response is characterized by the reaction of the cells, tissues and/or organs of an organism to the presence of a foreign substance. Such immune response typically leads to the production by the organism of one or more antibodies against the foreign substance, e.g., antigen or a portion of the antigen. Antigens of the invention may comprise a peptide, polypeptide, fusion protein, or any of the foregoing and may be conjugated or complexed to one or more separate adjuvants or heterologous proteins.

As used herein, an “adjuvant” is a pharmacological or immunological agent that modifies the effect of other agents. Adjuvants according to the present invention include, but are not limited chemical compositions, biomolecules, therapeutics, and/or therapeutic regimens.

In some embodiments, antigens of the invention comprise at least a portion of a prodomain of a TGF-beta family member and optionally one or more regions of intervening or flanking homologous or heterologous sequences. As used herein, “prodomain” means a region or section of a molecule preceding another. Often prodomains are synthesized as part of a larger protein and may play any number of roles from structural to functional, acting as a binding site, a protective partner, a signaling molecule, a trafficking entity, a stability enhancer, etc. The prodomains of the TGF-beta family members serve as unique antigens of the invention. Ranging in size from 200-450 amino acids, the entire prodomain or a portion thereof may be used as an antigen.

As used herein, a prodomain-derived antigen may comprise at least 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 250, 300, 350, 400, 450 or more amino acids selected from SEQ ID NOs. 1-37.

Intervening sequences which may be used as a component of an antigen may include homologous or heterologous sequences. These may be selected from other prodomains or from non-prodomain protein sequences.

Flanking sequences which may be used as a component of an antigen may include homologous or heterologous sequences. These may be selected from other prodomains or from non-prodomain protein sequences.

A list of exemplary TGF-beta family pro-proteins, i.e. the protein after removal of the secretion signal sequence, is shown in Table 1. The pro-protein contains, and is the precursor of, the prodomain and the growth factor. Shown in the Table are the names of the originating TGF-beta family member and the pro-protein sequence. Also identified in “bold” and “underlined” are furin cleavage sites. Upon furin cleavage, the resulting prodomain retains this site, whereas the mature growth factor begins following the furin cleavage site. It is noted that Lefty1 and Lefty2 are not cleaved. It is noted that some prodomains may be cleaved by furin (PACE) enzymes at additional sites, and by tolloid proteases. In some embodiments, pro-proteins may be cleaved at a first furin cleavage site (the first site being the site closest to the N-terminus). In some embodiments, pro-proteins may be cleaved at a furin cleavage site other than a first furin cleavage site.

TABLE 1
Pro-proteins of the TGF-beta family

| | | SEQ |
|---|---|---|
| TGF Member | Prodomain and growth factor Sequence | ID NO |
| TGF-beta 1 | LSTCKTIDMELVKRKRIEAIRGQILSKLRLASPPSQGEVPPGPLPEAV | 1 |
| | LALYNSTRDRVAGESAEPEPEPEADYYAKEVTRVLMVETHNEIYD | |
| | KFKQSTHSIYMFFNTSELREAVPEPVLLSRAELRLLRLKLKVEQHVE | |
| | LYQKYSNNSWRYLSNRLLAPSDSPEWLSFDVTGVVRQWLSRGGEI | |
| | EGFRLSAHCSCDSRDNTLQVDINGFTTGRRGDLATIHGMNRPFLLL | |
| | MATPLERAQHLQSSRHRRALDTNYCFSSTEKNCCVRQLYIDFRKD | |
| | LGWKWIHEPKGYHANFCLGPCPYIWSLDTQYSKVLALYNQHNPGA | |
| | SAAPCCVPQALEPLPIVYYVGRKPKVEQLSNMIVRSCKCS | |
| TGF-beta 2 | SLSTCSTLDMDQFMRKRIEAIRGQILSKLKLTSPPEDYPEPEEVPPEVI | 2 |
| | SIYNSTRDLLQEKASRRAAACERERSDEEYYAKEVYKIDMPPFFPSE | |
| | NAIPPTFYRPYFRIVRFDVSAMEKNASNLVKAEFRVFRLQNPKARV | |
| | PEQRIELYQILKSKDLTSPTQRYIDSKVVKTRAEGEWLSFDVTDAVH | |
| | EWLHHKDRNLGFKISLHCPCCTFVPSNNYIIPNKSEELEARFAGIDG | |
| | TSTYTSGDQKTIKSTRKKNSGKTPHLLLMLLPSYRLESQQTNRRKK | |
| | RALDAAYCFRNVQDNCCLRPLYIDFKRDLGWKWIHEPKGYNANFC | |
| | AGACPYLWSSDTQHSRVLSLYNTINPEASASPCCVSQDLEPLTILYY | |
| | IGKTPKIEQLSNMIVKSCKCS | |
| TGF-beta 3 | SLSLSTCTTLDFGHIKKKRVEAIRGQILSKLRLTSPPEPTVMTHVPYQ | 3 |
| | VLALYNSTRELLEEMHGEREEGCTQENTESEYYAKEIHKFDMIQGL | |
| | AEHNELAVCPKGITSKVFRFNVSSVEKNRTNLFRAEFRVLRVPNPSS | |
| | KRNEQRIELFQILRPDEHIAKQRYIGGKNLPTRGTAEWLSFDVTDTV | |
| | REWLLRRESNLGLEISIHCPCHTFQPNGDILENIHEVMEIKFKGVDNE | |
| | DDHGRGDLGRLKKQKDHHNPHLILMMIPPHRLDNPGQGGQRKKR | |
| | ALDTNYCFRNLEENCCVRPLYIDFRQDLGWKWVHEPKGYYANFCS | |
| | GPCPYLRSADTTHSTVLGLYNTLNPEASASPCCVPQDLEPLTILYYV | |
| | GRTPKVEQLSNMVVKSCKCS | |
| GDF11 | AEGPAAAAAAAAAAAAAGVGGERSSRPAPSVAPEPDGCPVCVWR | 4 |
| | QHSRELRLESIKSQILSKLRLKEAPNISREVVKQLLPKAPPLQQILDL | |
| | HDFQGDALQPEDFLEEDEYHATTETVISMAQETDPAVQTDGSPLCC | |
| | HFHFSPKVMFTKVLKAQLWVYLRPVPRPATVYLQILRLKPLTGEGT | |
| | AGGGGGGRRHIRIRSLKIELHSRSGHWQSIDFKQVLHSWFRQPQSN | |
| | WGIEINAFDPSGTDLAVTSLGPGAEGLHPFMELRVLENTKRSRRNL | |
| | GLDCDEHSSESRCCRYPLTVDFEAFGWDWIIAPKRYKANYCSGQCE | |
| | YMFMQKYPHTHLVQQANPRGSAGPCCTPTKMSPINMLYFNDKQQI | |
| | IYGKIPGMVVDRCGCS | |
| Myostatin | NENSEQKENVEKEGLCNACTWRQNTKSSRIEAIKIQILSKLRLETAP | 5 |
| (GDF8) | NISKDVIRQLLPKAPPLRELIDQYDVQRDDSSDGSLEDDDYHATTET | |
| | IITMPTESDFLMQVDGKPKCCFFKFSSKIQYNKVVKAQLWIYLRPVE | |
| | TPTTVFVQILRLIKPMKDGTRYTGIRSLKLDMNPGTGIWQSIDVKTV | |
| | LQNWLKQPESNLGIEIKALDENGHDLAVTFPGPGEDGLNPFLEVKV | |
| | TDTPKRSRRDFGLDCDEHSTESRCCRYPLTVDFEAFGWDWIIAPKR | |
| | YKANYCSGECEFVFLQKYPHTHLVHQANPRGSAGPCCTPTKMSPIN | |
| | MLYFNGKEQIIYGKIPAMVVDRCGCS | |
| Inhibin-beta A | SPTPGSEGHSAAPDCPSCALAALPKDVPNSQPEMVEAVKKHILNML | 6 |
| | HLKKRPDVTQPVPKAALLNAIRKLHVGKVGENGYVEIEDDIGRRAE | |
| | MNELMEQTSEIITFAESGTARKTLHFEISKEGSDLSVVERAEVWLFL | |
| | KVPKANRTRTKVTIRLFQQQKHPQGSLDTGEEAEEVGLKGERSELL | |
| | LSEKVVDARKSTWHVFPVSSSIQRLLDQGKSSLDVRIACEQCQESG | |
| | ASLVLLGKKKKKEEEGEGKKKGGGEGGAGADEEKEQSHRPFLML | |
| | QARQSEDHPHRRRRRGLECDGKVNICCKKQFFVSFKDIGWNDWII | |
| | APSGYHANYCEGECPSHIAGTSGSSLSFHSTVINHYRMRGHSPFANL | |
| | KSCCVPTKLRPMSMLYYDDGQNIIKKDIQNMIVEECGCS | |
| Inhibin-beta B | SPTPPPTPAAPPPPPPPGSPGGSQDTCTSCGGFRRPEELGRVDGDFLE | 7 |
| | AVKRHILSRLQMRGRPNITHAVPKAAMVTALRKLHAGKVREDGRV | |
| | EIPHLDGHASPGADGQERVSEIISFAETDGLASSRVRLYFFISNEGNQ | |
| | NLFVVQASLWLYLKLLPYVLEKGSRRKVRVKVYFQEQGHGDRWN | |
| | MVEKRVDLKRSGWHTFPLTEAIQALFERGERRLNLDVQCDSCQEL | |
| | AVVPVFVDPGEESHRPFVVVQARLGDSRHRIRKRGLECDGRTNLC | |
| | CRQQFFIDFRLIGWNDWIIAPTGYYGNYCEGSCPAYLAGVPGSASSF | |
| | HTAVVNQYRMRGLNPGTVNSCCIPTKLSTMSMLYFDDEYNIVKRD | |
| | VPNMIVEECGCA | |
| Inhibin-beta C | TPRAGGQCPACGGPTLELESQRELLLDLAKRSILDKLHLTQRPTLNR | 8 |
| | PVSRAALRTALQHLHGVPQGALLEDNREQECEIISFAETGLSTINQT | |
| | RLDFHFSSDRTAGDREVQQASLMFFVQLPSNTTWTLKVRVLVLGP | |
| | HNTNLTLATQYLLEVDASGWHQLPLGPEAQAACSQGHLTLELVLE | |
| | GQVAQSSVILGGAAHRPFVAARVRVGGKHQIHRRGIDCQGGSRMC | |
| | CRQEFFVDFREIGWHDWIIQPEGYAMNFCIGQCPLHIAGMPGIAASF | |
| | HTAVLNLLKANTAAGTTGGGSCCVPTARRPLSLLYYDRDSNIVKTD | |
| | IPDMVVEACGCS | |
| Inhibin-beta E | QGTGSVCPSCGGSKLAPQAERALVLELAKQQILDGLHLTSRPRITHP | 9 |
| | PPQAALTRALRRLQPGSVAPGNGEEVISFATVTDSTSAYSSLLTFHL | |
| | STPRSHHLYHARLWLHVLPTLPGTLCLRIFRWGPRRRRQGSRTLLA | |
| | EHHITNLGWHTLTLPSSGLRGEKSGVLKLQLDCRPLEGNSTVTGQP | |
| | RRLLDTAGHQQPFLELKIRANEPGAGRARRRTPTCEPATPLCCRRD | |
| | HYVDFQELGWRDWILQPEGYQLNYCSGQCPPHLAGSPGIAASFHSA | |
| | VFSLLKANNPWPASTSCCVPTARRPLSLLYLDHNGNVVKTDVPDM | |
| | VVEACGCS | |
| Lefty1 | LTGEQLLGSLLRQLQLKEVPTLDRADMEELVIPTHVRAQYVALLQR | 10 |
| | SHGDRSRGKRFSQSFREVAGRFLALEASTHLLVFGMEQRLPPNSEL | |
| | VQAVLRLFQEPVPKAALHRHGRLSPRSARARVTVEWLRVRDDGSN | |
| | RTSLIDSRLVSVHESGWKAFDVTEAVNFWQQLSRPRQPLLLQVSVQ | |
| | REHLGPLASGAHKLVRFASQGAPAGLGEPQLELHTLDLGDYGAQG | |
| | DCDPEAPMTEGTRCCRQEMYIDLQGMKWAENWVLEPPGFLAYEC | |
| | VGTCRQPPEALAFKWPFLGPRQCIASETDSLPMIVSIKEGGRTRPQV | |
| | VSLPNMRVQKCSCASDGALVPRRLQP | |
| Lefty2 | LTEEQLLGSLLRQLQLSEVPVLDRADMEKLVIPAHVRAQYVVLLRR | 11 |
| | SHGDRSRGKRFSQSFREVAGRFLASEASTHLLVFGMEQRLPPNSEL | |
| | VQAVLRLFQEPVPKAALHRHGRLSPRSAQARVTVEWLRVRDDGSN | |
| | RTSLIDSRLVSVHESGWKAFDVTEAVNFWQQLSRPRQPLLLQVSVQ | |
| | REHLGPLASGAHKLVRFASQGAPAGLGEPQLELHTLDLRDYGAQG | |
| | DCDPEAPMTEGTRCCRQEMYIDLQGMKWAKNWVLEPPGFLAYEC | |
| | VGTCQQPPEALAFNWPFLGPRQCIASETASLPMIVSIKEGGRTRPQV | |
| | VSLPNMRVQKCSCASDGALVPRRLQP | |
| GDF15 | LSLAEASRASFPGPSELHSEDSRFRELRKRYEDLLTRLRANQSWEDS | 12 |
| | NTDLVPAPAVRILTPEVRLGSGGHLHLRISRAALPEGLPEASRLHRA | |
| | LFRLSPTASRSWDVTRPLRRQLSLARPQAPALHLRLSPPPSQSDQLL | |
| | AESSSARPQLELHLRPQAARGRRRARARNGDHCPLGPGRCCRLHT | |
| | VRASLEDLGWADWVLSPREVQVTMCIGACPSQFRAANMHAQIKTS | |
| | LHRLKPDTVPAPCCVPASYNPMVLIQKTDTGVSLQTYDDLLAKDC | |
| | HCI | |
| Anti-Mullerian | LLGTEALRAEEPAVGTSGLIFREDLDWPPGIPQEPLCLVALGGDSNG | 13 |
| hormone | SSSPLRVVGALSAYEQAFLGAVQRARWGPRDLATFGVCNTGDRQA | |
| | ALPSLRRLGAWLRDPGGQRLVVLHLEEVTWEPTPSLRFQEPPPGGA | |
| | GPPELALLVLYPGPGPEVTVTRAGLPGAQSLCPSRDTRYLVLAVDR | |
| | PAGAWRGSGLALTLQPRGEDSRLSTARLQALLFGDDHRCFTRMTP | |
| | ALLLLPRSEPAPLPAHGQLDTVPFPPPRPSAELEESPPSADPFLETLTR | |
| | LVRALRVPPARASAPRLALDPDALAGFPQGLVNLSDPAALERLLDG | |
| | EEPLLLLLRPTAATTGDPAPLHDPTSAPWATALARRVAAELQAAAA | |
| | ELRSLPGLPPATAPLLARLLALCPGGPGGLGDPLRALLLLKALQGLR | |
| | VEWRGRDPRGPGRAQRSAGATAADGPCALRELSVDLRAERSVLIP | |
| | ETYQANNCQGVCGWPQSDRNPRYGNHVVLLLKMQVRGAALARPP | |
| | CCVPTAYAGKLLISLSEERISAHHVPNMVATECGCR | |
| Inhibin- | CQGLELARELVLAKVRALFLDALGPPAVTREGGDPGVRRLPRRHA | 14 |
| alpha | LGGFTHRGSEPEEEEDVSQAILFPATDASCEDKSAARGLAQEAEEGL | |
| | FRYMFRPSQHTRSRQVTSAQLWFHTGLDRQGTAASNSSEPLLGLLA | |
| | LSPGGPVAVPMSLGHAPPHWAVLHLATSALSLLTHPVLVLLLRCPL | |
| | CTCSARPEATPFLVAHTRTRPPSGGERARRSTPLMSWPWSPSALRL | |
| | LQRPPEEPAAHANCHRVALNISFQELGWERWIVYPPSFIFHYCHGG | |
| | CGLHIPPNLSLPVPGAPPTPAQPYSLLPGAQPCCAALPGTMRPLHVR | |
| | TTSDGGYSFKYETVPNLLTQHCACI | |
| GDF1 | PVPPGPAAALLQALGLRDEPQGAPRLRPVPPVMWRLFRRRDPQETR | 15 |
| | SGSRRTSPGVTLQPCHVEELGVAGNIVRHIPDRGAPTRASEPASAAG | |
| | HCPEWTVVFDLSAVEPAERPSRARLELRFAAAAAAAPEGGWELSV | |
| | AQAGQGAGADPGPVLLRQLVPALGPPVRAELLGAAWARNASWPR | |
| | SLRLALALRPRAPAACARLAEASLLLVTLDPRLCHPLARPRRDAEP | |
| | VLGGGPGGACRARRLYVSFREVGWHRWVIAPRGFLANYCQGQCA | |
| | LPVALSGSGGPPALNHAVLRALMHAAAPGAADLPCCVPARLSPISV | |
| | LFFDNSDNVVLRQYEDMVVDECGCR | |
| GDF3 | QEYVFLQFLGLDKAPSPQKFQPVPYILKKIFQDREAAATTGVSRDLC | 16 |
| | YVKELGVRGNVLRFLPDQGFFLYPKKISQASSCLQKLLYFNLSAIKE | |
| | REQLTLAQLGLDLGPNSYYNLGPELELALFLVQEPHVWGQTTPKPG | |
| | KMFVLRSVPWPQGAVHFNLLDVAKDWNDNPRKNFGLFLEILVKED | |
| | RDSGVNFQPEDTCARLRCSLHASLLVVTLNPDQCHPSRKRRAAIPV | |
| | PKLSCKNLCHRHQLFINFRDLGWHKWIIAPKGFMANYCHGECPFSL | |
| | TISLNSSNYAFMQALMHAVDPEIPQAVCIPTKLSPISMLYQDNNDNV | |
| | ILRHYEDMVVDECGCG | |
| GDF5 | APDLGQRPQGTRPGLAKAEAKERPPLARNVFRPGGHSYGGGATNA | 17 |
| | NARAKGGTGQTGGLTQPKKDEPKKLPPRPGGPEPKPGHPPQTRQAT | |
| | ARTVTPKGQLPGGKAPPKAGSVPSSFLLKKAREPGPPREPKEPFRPP | |
| | PITPHEYMLSLYRTLSDADRKGGNSSVKLEAGLANTITSFIDKGQDD | |
| | RGPVVRKQRYVFDISALEKDGLLGAELRILRKKPSDTAKPAAPGGG | |
| | RAAQLKLSSCPSGRQPASLLDVRSVPGLDGSGWEVFDIWKLFRNFK | |
| | NSAQLCLELEAWERGRAVDLRGLGFDRAARQVHEKALFLVFGRTK | |
| | KRDLFFNEIKARSGQDDKTVYEYLFSQRRKRRAPLATRQGKRPSK | |
| | NLKARCSRKALHVNFKDMGWDDWIIAPLEYEAFHCEGLCEFPLRS | |
| | HLEPTNHAVIQTLMNSMDPESTPPTCCVPTRLSPISILFIDSANNVVY | |
| | KQYEDMVVESCGCR | |
| GDF6 | FQQASISSSSSSAELGSTKGMRSRKEGKMQRAPRDSDAGREGQEPQ | 18 |
| | PRPQDEPRAQQPRAQEPPGRGPRVVPHEYMLSIYRTYSIAEKLGINA | |
| | SFFQSSKSANTITSFVDRGLDDLSHTPLRRQKYLFDVSMLSDKEELV | |
| | GAELRLFRQAPSAPWGPPAGPLHVQLFPCLSPLLLDARTLDPQGAPP | |
| | AGWEVFDVWQGLRHQPWKQLCLELRAAWGELDAGEAEARARGP | |
| | QQPPPPDLRSLGFGRRVRPPQERALLVVFTRSQRKNLFAEMREQLG | |
| | SAEAAGPGAGAEGSWPPPSGAPDARPWLPSPGRRRRRTAFASRHG | |
| | KRHGKKSRLRCSKKPLHVNFKELGWDDWIIAPLEYEAYHCEGVCD | |
| | FPLRSHLEPTNHAIIQTLMNSMDPGSTPPSCCVPTKLTPISILYIDAGN | |
| | NVVYKQYEDMVVESCGCR | |
| GDF7 | RDGLEAAAVLRAAGAGPVRSPGGGGGGGGGGRTLAQAAGAAAVP | 19 |
| | AAAVPRARAARRAAGSGFRNGSVVPHHFMMSLYRSLAGRAPAGA | |
| | AAVSASGHGRADTITGFTDQATQDESAAETGQSFLFDVSSLNDADE | |
| | VVGAELRVLRRGSPESGPGSWTSPPLLLLSTCPGAARAPRLLYSRA | |
| | AEPLVGQRWEAFDVADAMRRHRREPRPPRAFCLLLRAVAGPVPSP | |
| | LALRRLGFGWPGGGGSAAEERAVLVVSSRTQRKESLFREIRAQARA | |
| | LGAALASEPLPDPGTGTASPRAVIGGRRRRRTALAGTRTAQGSGGG | |
| | AGRGHGRRGRSRCSRKPLHVDFKELGWDDWIIAPLDYEAYHCEGL | |
| | CDFPLRSHLEPTNHAIIQTLLNSMAPDAAPASCCVPARLSPISILYIDA | |
| | ANNVVYKQYEDMVVEACGCR | |
| BMP10 | SPIMNLEQSPLEEDMSLFGDVFSEQDGVDFNTLLQSMKDEFLKTLN | 20 |
| | LSDIPTQDSAKVDPPEYMLELYNKFATDRTSMPSANIIRSFKNEDLF | |
| | SQPVSFNGLRKYPLLFNVSIPHHEEVIMAELRLYTLVQRDRMIYDG | |
| | VDRKITIFEVLESKGDNEGERNMLVLVSGEIYGTNSEWETFDVTDAI | |
| | RRWQKSGSSTHQLEVHIESKHDEAEDASSGRLEIDTSAQNKHNPLLI | |
| | VFSDDQSSDKERKEELNEMISHEQLPELDNLGLDSFSSGPGEEALLQ | |
| | MRSNIIYDSTARIRRNAKGNYCKRTPLYIDFKEIGWDSWIIAPPGYE | |
| | AYECRGVCNYPLAEHLTPTKHAIIQALVHLKNSQKASKACCVPTKL | |
| | EPISILYLDKGVVTYKFKYEGMAVSECGCR | |
| BMP9 (GDF2) | KPLQSWGRGSAGGNAHSPLGVPGGGLPEHTFNLKMFLENVKVDFL | 21 |
| | RSLNLSGVPSQDKTRVEPPQYMIDLYNRYTSDKSTTPASNIVRSFSM | |
| | EDAISITATEDFPFQKHILLFNISIPRHEQITRAELRLYVSCQNHVDPS | |
| | HDLKGSVVIYDVLDGTDAWDSATETKTFLVSQDIQDEGWETLEVS | |
| | SAVKRWVRSDSTKSKNKLEVTVESHRKGCDTLDISVPPGSRNLPFF | |
| | VVFSNDHSSGTKETRLELREMISHEQESVLKKLSKDGSTEAGESSHE | |
| | EDTDGHVAAGSTLARRKRSAGAGSHCQKTSLRVNFEDIGWDSWII | |
| | APKEYEAYECKGGCFFPLADDVTPTKHAIVQTLVHLKFPTKVGKAC | |
| | CVPTKLSPISVLYKDDMGVPTLKYHYEGMSVAECGCR | |
| Noda1 | TVATALLRTRGQPSSPSPLAYMLSLYRDPLPRADIIRSLQAEDVAVD | 22 |
| | GQNWTFAFDFSFLSQQEDLAWAELRLQLSSPVDLPTEGSLAIEIFHQ | |
| | PKPDTEQASDSCLERFQMDLFTVTLSQVTFSLGSMVLEVTRPLSKW | |
| | LKRPGALEKQMSRVAGECWPRPPTPPATNVLLMLYSNLSQEQRQL | |
| | GGSTLLWEAESSWRAQEGQLSWEWGKRHRRHHLPDRSQLCRKVK | |
| | FQVDFNLIGWGSWIIYPKQYNAYRCEGECPNPVGEEFHPTNHAYIQ | |
| | SLLKRYQPHRVPSTCCAPVKTKPLSMLYVDNGRVLLDHHKDMIVE | |
| | ECGCL | |
| BMP2 | LVPELGRRKFAAASSGRPSSQPSDEVLSEFELRLLSMFGLKQRPTPS | 23 |
| | RDAVVPPYMLDLYRRHSGQPGSPAPDHRLERAASRANTVRSFHHE | |
| | ESLEELPETSGKTTRRFFFNLSSIPTEEFITSAELQVFREQMQDALGN | |
| | NSSFHHRINIYEIIKPATANSKFPVTRLLDTRLVNQNASRWESFDVTP | |
| | AVMRWTAQGHANHGFVVEVAHLEEKQGVSKRHVRISRSLHQDEH | |
| | SWSQIRPLLVTFGHDGKGHPLHKREKRQAKHKQRKRLKSSCKRHP | |
| | LYVDFSDVGWNDWIVAPPGYHAFYCHGECPFPLADHLNSTNHAIV | |
| | QTLVNSVNSKIPKACCVPTELSAISMLYLDENEKVVLKNYQDMVV | |
| | EGCGCR | |
| BMP4 | GASHASLIPETGKKKVAEIQGHAGGRRSGQSHELLRDFEATLLQMF | 24 |
| | GLRRRPQPSKSAVIPDYMRDLYRLQSGEEEEEQIHSTGLEYPERPAS | |
| | RANTVRSFHHEEHLENIPGTSENSAFRFLFNLSSIPENEVISSAELRLF | |
| | REQVDQGPDWERGFHRINIYEVMKPPAEVVPGHLITRLLDTRLVHH | |
| | NVTRWETFDVSPAVLRWTREKQPNYGLAIEVTHLHQTRTHQGQHV | |
| | RISRSLPQGSGNWAQLRPLLVTFGHDGRGHALTRRRRAKRSPKHH | |
| | SQRARKKNKNCRRHSLYVDFSDVGWNDWIVAPPGYQAFYCHGDC | |
| | PFPLADHLNSTNHAIVQTLVNSVNSSIPKACCVPTELSAISMLYLDE | |
| | YDKVVLKNYQEMVVEGCGCR | |
| BMP5 | DNHVHSSFIYRRLRNHERREIQREILSILGLPHRPRPFSPGKQASSAPL | 25 |
| | FMLDLYNAMTNEENPEESEYSVRASLAEETRGARKGYPASPNGYP | |
| | RRIQLSRTTPLTTQSPPLASLHDTNFLNDADMVMSFVNLVERDKDF | |
| | SHQRRHYKEFRFDLTQIPHGEAVTAAEFRIYKDRSNNRFENETIKISI | |
| | YQIIKEYTNRDADLFLLDTRKAQALDVGWLVFDITVTSNHWVINPQ | |
| | NNLGLQLCAETGDGRSINVKSAGLVGRQGPQSKQPFMVAFFKASE | |
| | VLLRSVRAANKRKNQNRNKSSSHQDSSRMSSVGDYNTSEQKQAC | |
| | KKHELYVSFRDLGWQDWIIAPEGYAAFYCDGECSFPLNAHMNATN | |
| | HAIVQTLVHLMFPDHVPKPCCAPTKLNAISVLYFDDSSNVILKKYR | |
| | NMVVRSCGCH | |
| BMP6 | CCGPPPLRPPLPAAAAAAAGGQLLGDGGSPGRTEQPPPSPQSSSGFL | 26 |
| | YRRLKTQEKREMQKEILSVLGLPHRPRPLHGLQQPQPPALRQQEEQ | |
| | QQQQQLPRGEPPPGRLKSAPLFMLDLYNALSADNDEDGASEGERQ | |
| | QSWPHEAASSSQRRQPPPGAAHPLNRKSLLAPGSGSGGASPLTSAQ | |
| | DSAFLNDADMVMSFVNLVEYDKEFSPRQRHHKEFKFNLSQIPEGEV | |
| | VTAAEFRIYKDCVMGSFKNQTFLISIYQVLQEHQHRDSDLFLLDTR | |
| | VVWASEEGWLEFDITATSNLWVVTPQHNMGLQLSVVTRDGVHVH | |
| | PRAAGLVGRDGPYDKQPFMVAFFKVSEVHVRTTRSASSRRRQQSR | |
| | NRSTQSQDVARVSSASDYNSSELKTACRKHELYVSFQDLGWQDWII | |
| | APKGYAANYCDGECSFPLNAHMNATNHAIVQTLVHLMNPEYVPKP | |
| | CCAPTKLNAISVLYFDDNSNVILKKYRNMVVRACGCH | |
| BMP7 | DFSLDNEVHSSFIHRRLRSQERREMQREILSILGLPHRPRPHLQGKH | 27 |
| | NSAPMFMLDLYNAMAVEEGGGPGGQGFSYPYKAVFSTQGPPLASL | |
| | QDSHFLTDADMVMSFVNLVEHDKEFFHPRYHHREFRFDLSKIPEGE | |
| | AVTAAEFRIYKDYIRERFDNETFRISVYQVLQEHLGRESDLFLLDSR | |
| | TLWASEEGWLVFDITATSNHWVVNPRHNLGLQLSVETLDGQSINP | |
| | KLAGLIGRHGPQNKQPFMVAFFKATEVHFRSIRSTGSKQRSQNRSK | |
| | TPKNQEALRMANVAENSSSDQRQACKKHELYVSFRDLGWQDWIIA | |
| | PEGYAAYYCEGECAFPLNSYMNATNHAIVQTLVHFINPETVPKPCC | |
| | APTQLNAISVLYFDDSSNVILKKYRNMVVRACGCH | |
| BMP8A | GGGPGLRPPPGCPQRRLGARERRDVQREILAVLGLPGRPRPRAPPA | 28 |
| | ASRLPASAPLFMLDLYHAMAGDDDEDGAPAEQRLGRADLVMSFV | |
| | NMVERDRALGHQEPHWKEFRFDLTQIPAGEAVTAAEFRIYKVPSIH | |
| | LLNRTLHVSMFQVVQEQSNRESDLFFLDLQTLRAGDEGWLVLDVT | |
| | AASDCWLLKRHKDLGLRLYVETEDGHSVDPGLAGLLGQRAPRSQQ | |
| | PFVVTFFRASPSPIRTPRAVRPLRRRQPKKSNELPQANRLPGIFDDV | |
| | RGSHGRQVCRRHELYVSFQDLGWLDWVIAPQGYSAYYCEGECSFP | |
| | LDSCMNATNHAILQSLVHLMKPNAVPKACCAPTKLSATSVLYYDS | |
| | SNNVILRKHRNMVVKACGCH | |
| BMP8B | GGGPGLRPPPGCPQRRLGARERRDVQREILAVLGLPGRPRPRAPPA | 29 |
| | ASRLPASAPLFMLDLYHAMAGDDDEDGAPAERRLGRADLVMSFV | |
| | NMVERDRALGHQEPHWKEFRFDLTQIPAGEAVTAAEFRIYKVPSIH | |
| | LLNRTLHVSMFQVVQEQSNRESDLFFLDLQTLRAGDEGWLVLDVT | |
| | AASDCWLLKRHKDLGLRLYVETEDGHSVDPGLAGLLGQRAPRSQQ | |
| | PFVVTFFRASPSPIRTPRAVRPLRRRQPKKSNELPQANRLPGIFDDV | |
| | HGSHGRQVCRRHELYVSFQDLGWLDWVIAPQGYSAYYCEGECSFP | |
| | LDSCMNATNHAILQSLVHLMMPDAVPKACCAPTKLSATSVLYYDS | |
| | SNNVILRKHRNMVVKACGCH | |
| BMP15 | MEHRAQMAEGGQSSIALLAEAPTLPLIEELLEESPGEQPRKPRLLGH | 30 |
| | SLRYMLELYRRSADSHGHPRENRTIGATMVRLVKPLTSVARPHRGT | |
| | WHIQILGFPLRPNRGLYQLVRATVVYRHHLQLTRFNLSCHVEPWV | |
| | QKNPTNHFPSSEGDSSKPSLMSNAWKEMDITQLVQQRFWNNKGHR | |
| | ILRLRFMCQQQKDSGGLELWHGTSSLDIAFLLLYFNDTHKSIRKAKF | |
| | LPRGMEEFMERESLLRRTRQADGISAEVTASSSKHSGPENNQCSLH | |
| | PFQISFRQLGWDHWIIAPPFYTPNYCKGTCLRVLRDGLNSPNHAIIQ | |
| | NLINQLVDQSVPRPSCVPYKYVPISVLMIEANGSILYKEYEGMIAES | |
| | CTCR | |
| GDF9 | SQASGGEAQIAASAELESGAMPWSLLQHIDERDRAGLLPALFKVLS | 31 |
| | VGRGGSPRLQPDSRALHYMKKLYKTYATKEGIPKSNRSHLYNTVR | |
| | LFTPCTRHKQAPGDQVTGILPSVELLFNLDRITTVEHLLKSVLLYNIN | |
| | NSVSFSSAVKCVCNLMIKEPKSSSRTLGRAPYSFTFNSQFEFGKKHK | |
| | WIQIDVTSLLQPLVASNKRSIHMSINFTCMKDQLEHPSAQNGLFNM | |
| | TLVSPSLILYLNDTSAQAYHSWYSLHYKRRPSQGPDQERSLSAYPV | |
| | GEEAAEDGRSSHHRHRRGQETVSSELKKPLGPASFNLSEYFRQFLL | |
| | PQNECELHDFRLSFSQLKWDNWIVAPHRYNPRYCKGDCPRAVGHR | |
| | YGSPVHTMVQNIIYEKLDSSVPRPSCVPAKYSPLSVLTIEPDGSIAYK | |
| | EYEDMIATKCTCR | |
| BMP3 | ERPKPPFPELRKAVPGDRTAGGGPDSELQPQDKVSEHMLRLYDRYS | 32 |
| | TVQAARTPGSLEGGSQPWRPRLLREGNTVRSFRAAAAETLERKGL | |
| | YIFNLTSLTKSENILSATLYFCIGELGNISLSCPVSGGCSHHAQRKHIQ | |
| | IDLSAWTLKFSRNQSQLLGHLSVDMAKSHRDIMSWLSKDITQLLRK | |
| | AKENEEFLIGFNITSKGRQLPKRRLPFPEPYILVYANDAAISEPESVV | |
| | SSLQGHRNFPTGTVPKWDSHIRAALSIERRKKRSTGVLLPLQNNELP | |
| | GAEYQYKKDEVWEERKPYKTLQAQAPEKSKNKKKQRKGPHRKS | |
| | QTLQFDEQTLKKARRKQWIEPRNCARRYLKVDFADIGWSEWIISPK | |
| | SFDAYYCSGACQFPMPKSLKPSNHATIQSIVRAVGVVPGIPEPCCVP | |
| | EKMSSLSILFFDENKNVVLKVYPNMTVESCACR | |
| GDF10 | SHRAPAWSALPAAADGLQGDRDLQRHPGDAAATLGPSAQDMVAV | 33 |
| | HMHRLYEKYSRQGARPGGGNTVRSFRARLEVVDQKAVYFFNLTS | |
| | MQDSEMILTATFHFYSEPPRWPRALEVLCKPRAKNASGRPLPLGPP | |
| | TRQHLLFRSLSQNTATQGLLRGAMALAPPPRGLWQAKDISPIVKAA | |
| | RRDGELLLSAQLDSEERDPGVPRPSPYAPYILVYANDLAISEPNSVA | |
| | VTLQRYDPFPAGDPEPRAAPNNSADPRVRRAAQATGPLQDNELPGL | |
| | DERPPRAHAQHFHKHQLWPSPFRALKPRPGRKDRRKKGQEVFMAA | |
| | SQVLDFDEKTMQKARRKQWDEPRVCSRRYLKVDFADIGWNEWIIS | |
| | PKSFDAYYCAGACEFPMPKIVRPSNHATIQSIVRAVGIIPGIPEPCCVP | |
| | DKMNSLGVLFLDENRNVVLKVYPNMSVDTCACR | |
| GDNF | FPLPAGKRPPEAPAEDRSLGRRRAPFALSSDSNMPEDYPDQFDDVM | 34 |
| | DFIQATIKRLKRSPDKQMAVLPRRERNRQAAAANPENSRGKGRRG | |
| | QRGKNRGCVLTAIHLNVTDLGLGYETKEELIFRYCSGSCDAAETTY | |
| | DKILKNLSRNRRLVSDKVGQACCRPIAFDDDLSFLDDNLVYHILRK | |
| | HSAKRCGCI | |
| NRTN | IWMCREGLLLSHRLGPALVPLHRLPRTLDARIARLAQYRALLQGAP | 35 |
| | DAMELRELTPWAGRPPGPRRRAGPRRRRARARLGARPCGLRELEV | |
| | RVSELGLGYASDETVLFRYCAGACEAAARVYDLGLRRLRQRRRLR | |
| | RERVRAQPCCRPTAYEDEVSFLDAHSRYHTVHELSARECACV | |
| PSPN | WGPDARGVPVADGEFSSEQVAKAGGTWLGTHRPLARLRRALSGP | 36 |
| | CQLWSLTLSVAELGLGYASEEKVIFRYCAGSCPRGARTQHGLALAR | |
| | LQGQGRAHGGPCCRPTRYTDVAFLDDRHRWQRLPQLSAAACGCG | |
| | G | |
| ARTN | SLGSAPRSPAPREGPPPVLASPAGHLPGGRTARWCSGRARRPPPQPS | 37 |
| | RPAPPPPAPPSALPRGGRAARAGGPGSRARAAGARGCRLRSQLVP | |
| | VRALGLGHRSDELVRFRFCSGSCRRARSPHDLSLASLLGAGALRPPP | |
| | GSRPVSQPCCRPTRYEAVSFMDVNSTWRTVDRLSATACGCLG | |

According to the present invention, the entire GPC, or the pro-proteins listed in Table 1, may be used as antigens. Alternatively regions of the prodomains may be used. In some embodiments, fragments generated after cleavage at furin cleavage sites may be used. In some embodiments, variants lacking one or more furin cleavage site may be used as antigens. In some embodiments, variants comprising mutated furin cleavage sites may be used as antigens. In some embodiments, regions comprising the alpha-1 helix region may be used. Table 2 lists the alpha-1 helix region of each of the prodomains. In some embodiments, mutants of any of the aforementioned may be used as antigens. Such mutants may include cysteine mutants that are incapable of binding with ECCM components including, but not limited to LTBP and GARP.

TABLE 2
Alpha-1 Regions as Antigens

| | | SEQ |
|---|---|---|
| TGF Member | Alpha-1 Region Sequence | ID NO |
| TGF-beta 1 | LSTCKTIDMELVKRKRIEAIRGQILSKLRL | 38 |
| TGF-beta 2 | LSTCSTLDMDQFMRKRIEAIRGQILSKLKL | 39 |
| TGF-beta 3 | LSTCTTLDFGHIKKKRVEAIRGQILSKLRL | 40 |
| GDF11 | DGCPVCVWRQHSRELRLESIKSQILSKLRL | 41 |
| Myostatin | GLCNACTWRQNTKSSRIEAIKIQILSKLRL | 42 |
| (GDF 8) | | |
| Inhibin-beta A | ALAALPKDVPNSQPEMVEAVKKHILNMLHL | 43 |
| Inhibin-beta B | GGFRRPEELGRVDGDFLEAVKRHILSRLQM | 44 |
| Inhibin-beta C | GGPTLELESQRELLLDLAKRSILDKLHL | 45 |
| Inhibin-beta E | GGSKLAPQAERALVLELAKQQILDGLHL | 46 |
| Lefty1 | LTGEQLLGSLLRQLQL | 47 |
| Lefty2 | LTEEQLLGSLLRQLQL | 48 |
| GDF15 | LSL | 49 |
| Anti-Mullerian | GAWLRDPGGQRLVVLHLEEVTWEPTPSLRF | 50 |
| hormone | | |
| Inhibin-alpha | CQGLELARELVLAKVRALFLDAL | 51 |
| GDF1 | PVPPGPAAALLQALGL | 52 |
| GDF3 | QEYVFLQFLGL | 53 |
| GDF5 | VTPKGQLPGGKAPPKAGSVPSSFLLKKARE | 54 |
| GDF6 | KEGKMQRAPRDSDAGREGQEPQPRPQDEPR | 55 |
| GDF7 | RAAGAGPVRSPGGGGGGGGGGRTLAQAAGA | 56 |
| BMP10 | FGDVFSEQDGVDFNTLLQSMKDEFLKTLNL | 57 |
| BMP9 (GDF2) | VPGGGLPEHTFNLKMFLENVKVDFLRSLNL | 58 |
| Noda1 | TVATALLRTRGQ | 59 |
| BMP2 | AAASSGRPSSQPSDEVLSEFELRLLSMFGL | 60 |
| BMP4 | QGHAGGRRSGQSHELLRDFEATLLQMFGL | 61 |
| BMP5 | NHVHSSFIYRRLRNHERREIQREILSILGL | 62 |
| BMP6 | PQSSSGFLYRRLKTQEKREMQKEILSVLGL | 63 |
| BMP7 | NEVHSSFIHRRLRSQERREMQREILSILGL | 64 |
| BMP8A/BMP8B | LRPPPGCPQRRLGARERRDVQREILAVLGL | 65 |
| BMP15 | HRAQMAEGGQSSIALLAEAPTLPLIEELL | 66 |
| GDF9 | AELESGAMPWSLLQHIDERDRAGLLPALFK | 67 |
| BMP3 | ERPKPPFPELRKAVPGDRTA | 68 |
| GDF10 | SHRAPAWSALPAAADGLQGDRDL | 69 |
| GDNF | FPLPAGKRPPEAPAEDRSLGR | 70 |
| NRTN | HRLGPALVPLHRLPRTLDARIARLAQYRAL | 71 |
| PSPN | WGPDA | 72 |
| ARTN | PREGPPPVLASPAGHLPGGRTARWCSGRAR | 73 |

Antibodies of the present invention, as well as antigens used to generate them, are primarily amino acid-based molecules. These molecules may be “peptides,” “polypeptides,” or “proteins.”

As used herein, the term “peptide” refers to an amino-acid based molecule having from 2 to 50 or more amino acids. Special designators apply to the smaller peptides with “dipeptide” referring to a two amino acid molecule and “tripeptide” referring to a three amino acid molecule. Amino acid based molecules having more than 50 contiguous amino acids are considered polypeptides or proteins.

The terms “amino acid” and “amino acids” refer to all naturally occurring L-alpha-amino acids as well as non-naturally occurring amino acids. Amino acids are identified by either the one-letter or three-letter designations as follows: aspartic acid (Asp:D), isoleucine (Ile:I), threonine (Thr:T), leucine (Leu:L), serine (Ser:S), tyrosine (Tyr:Y), glutamic acid (Glu:E), phenylalanine (Phe:F), proline (Pro:P), histidine (His:H), glycine (Gly:G), lysine (Lys:K), alanine (Ala:A), arginine (Arg:R), cysteine (Cys:C), tryptophan (Trp:W), valine (Val:V), glutamine (Gln:Q) methionine (Met:M), asparagines (Asn:N), where the amino acid is listed first followed parenthetically by the three and one letter codes, respectively.

### Growth Factor Directed Agents (GDAs): Variations

GDAs of the present invention may exist as a whole polypeptide, a plurality of polypeptides or fragments of polypeptides, which independently may be encoded by one or more nucleic acids, a plurality of nucleic acids, fragments of nucleic acids or variants of any of the aforementioned. As used herein, “polypeptide” means a polymer of amino acid residues (natural or unnatural) linked together most often by peptide bonds. The term, as used herein, refers to proteins, polypeptides, and peptides of any size, structure, or function. In some instances the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and preferably, they will be at least about 80%, more preferably at least about 90% identical (homologous) to a native or reference sequence.

In some embodiments “variant mimics” are provided. As used herein, the term “variant mimic” is one which contains one or more amino acids which would mimic an activated sequence. For example, glutamate may serve as a mimic for phosphoro-threonine and/or phosphoro-serine. Alternatively, variant mimics may result in deactivation or in an inactivated product containing the mimic, e.g., phenylalanine may act as an inactivating substitution for tyrosine; or alanine may act as an inactivating substitution for serine. The amino acid sequences of the GDAs of the invention may comprise naturally occurring amino acids and as such may be considered to be proteins, peptides, polypeptides, or fragments thereof. Alternatively, the GDAs may comprise both naturally and non-naturally occurring amino acids.

The term “amino acid sequence variant” refers to molecules with some differences in their amino acid sequences as compared to a native or starting sequence. The amino acid sequence variants may possess substitutions, deletions, and/or insertions at certain positions within the amino acid sequence. “Native” or “starting” sequence should not be confused with a wild type sequence. As used herein, a native or starting sequence is a relative term referring to an original molecule against which a comparison may be made. “Native” or “starting” sequences or molecules may represent the wild-type (that sequence found in nature) but do not have to be the wild-type sequence.

Ordinarily, variants will possess at least about 70% homology to a native sequence, and preferably, they will be at least about 80%, more preferably at least about 90% homologous to a native sequence.

“Homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

By “homologs” as it applies to amino acid sequences is meant the corresponding sequence of other species having substantial identity to a second sequence of a second species.

“Analogs” is meant to include polypeptide variants which differ by one or more amino acid alterations, e.g., substitutions, additions or deletions of amino acid residues that still maintain the properties of the parent polypeptide.

The term “derivative” is used synonymously with the term “variant” and refers to a molecule that has been modified or changed in any way relative to a reference molecule or starting molecule.

The present invention contemplates several types of GDAs which are amino acid based including variants and derivatives. These include substitutional, insertional, deletion and covalent variants and derivatives. As such, included within the scope of this invention are GDA molecules containing substitutions, insertions and/or additions, deletions and covalently modifications. For example, sequence tags or amino acids, such as one or more lysines, can be added to the peptide sequences of the invention (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to proteins are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Insertional variants” when referring to proteins are those with one or more amino acids inserted immediately adjacent to an amino acid at a particular position in a native or starting sequence. “Immediately adjacent” to an amino acid means connected to either the alpha-carboxy or alpha-amino functional group of the amino acid.

“Deletional variants” when referring to proteins, are those with one or more amino acids in the native or starting amino acid sequence removed. Ordinarily, deletional variants will have one or more amino acids deleted in a particular region of the molecule.

The term “derivatives,” as referred to herein includes modifications of a native or starting protein with an organic proteinaceous or non-proteinaceous derivatizing agent, and post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the proteins used in accordance with the present invention.

Other post-translational modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)).

Covalent derivatives specifically include fusion molecules in which proteins of the invention are covalently bonded to a non-proteinaceous polymer. The non-proteinaceous polymer ordinarily is a hydrophilic synthetic polymer, i.e. a polymer not otherwise found in nature. However, polymers which exist in nature and are produced by recombinant or in vitro methods are useful, as are polymers which are isolated from nature. Hydrophilic polyvinyl polymers fall within the scope of this invention, e.g. polyvinylalcohol and polyvinylpyrrolidone. Particularly useful are polyvinylalkylene ethers such a polyethylene glycol, polypropylene glycol. The proteins may be linked to various non-proteinaceous polymers, such as polyethylene glycol, polypropylene glycol or polyoxyalkylenes, in the manner set forth in U.S. Pat. Nos. 4,640,835; 4,496,689; 4,301,144; 4,670,417; 4,791,192 or 4,179,337.

“Features” when referring to proteins are defined as distinct amino acid sequence-based components of a molecule. Features of the proteins of the present invention include surface manifestations, local conformational shape, folds, loops, half-loops, domains, half-domains, sites, termini or any combination thereof.

As used herein when referring to proteins the term “surface manifestation” refers to a polypeptide based component of a protein appearing on an outermost surface.

As used herein when referring to proteins the term “local conformational shape” means a polypeptide based structural manifestation of a protein which is located within a definable space of the protein.

As used herein when referring to proteins the term “fold” means the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

As used herein the term “turn” as it relates to protein conformation means a bend which alters the direction of the backbone of a peptide or polypeptide and may involve one, two, three or more amino acid residues.

As used herein when referring to proteins the term “loop” refers to a structural feature of a peptide or polypeptide which reverses the direction of the backbone of a peptide or polypeptide and comprises four or more amino acid residues. Oliva et al. have identified at least 5 classes of protein loops (J. Mol Biol 266 (4): 814-830; 1997).

As used herein when referring to proteins the term “half-loop” refers to a portion of an identified loop having at least half the number of amino acid resides as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids). For example, a loop identified as a 7 amino acid loop could produce half-loops of 3 amino acids or 4 amino acids (7/2=3.5+/−0.5 being 3 or 4).

As used herein when referring to proteins the term “domain” refers to a motif of a polypeptide having one or more identifiable structural or functional characteristics or properties (e.g., binding capacity, serving as a site for protein-protein interactions.

As used herein when referring to proteins the term “half-domain” means portion of an identified domain having at least half the number of amino acid resides as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5+/−0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein when referring to proteins the terms “site” as it pertains to amino acid based embodiments is used synonymous with “amino acid residue” and “amino acid side chain”. A site represents a position within a peptide or polypeptide that may be modified, manipulated, altered, derivatized or varied within the polypeptide based molecules of the present invention.

As used herein the terms “termini or terminus” when referring to proteins refers to an extremity of a peptide or polypeptide. Such extremity is not limited only to the first or final site of the peptide or polypeptide but may include additional amino acids in the terminal regions. The polypeptide based molecules of the present invention may be characterized as having both an N-terminus (terminated by an amino acid with a free amino group (NH2)) and a C-terminus (terminated by an amino acid with a free carboxyl group (COOH)). Proteins of the invention are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide based moiety such as an organic conjugate.

Once any of the features have been identified or defined as a component of a molecule of the invention, any of several manipulations and/or modifications of these features may be performed by moving, swapping, inverting, deleting, randomizing or duplicating. Furthermore, it is understood that manipulation of features may result in the same outcome as a modification to the molecules of the invention. For example, a manipulation which involved deleting a domain would result in the alteration of the length of a molecule just as modification of a nucleic acid to encode less than a full length molecule would.

Modifications and manipulations can be accomplished by methods known in the art such as site directed mutagenesis. The resulting modified molecules may then be tested for activity using in vitro or in vivo assays such as those described herein or any other suitable screening assay known in the art.

### Isotopic Variations

The GDAs of the present invention may contain one or more atoms that are isotopes. As used herein, the term “isotope” refers to a chemical element that has one or more additional neutron. In one embodiment, compounds of the present invention may be deuterated. As used herein, the term “deuterated” refers to a substance that has had one or more hydrogen atoms replaced by deuterium isotopes. Deuterium isotopes are isotopes of hydrogen. The nucleus of hydrogen contains one proton while deuterium nuclei contain both a proton and a neutron. The GDAs may be deuterated in order to change a physical property of the compound, such as stability, or to allow the compounds to be used in diagnostic and experimental applications.

### Growth Factor Directed Agents (GDAs): Conjugates and Combinations

It is contemplated by the present invention that the GDAs, antigens and/or antibodies of the present invention may be complexed, conjugated or combined with one or more homologous or heterologous molecules. As used herein, “homologous molecule” means a molecule which is similar in at least one of structure or function relative to a starting molecule while a “heterologous molecule” is one that differs in at least one of structure or function relative to a starting molecule. Structural homologs are therefore molecules which are substantially structurally similar. They can be identical. Functional homologs are molecules which are substantially functionally similar. They can be identical.

GDAs of the invention may comprise conjugates. Such conjugates of the invention may include a naturally occurring substance or ligand, such as a protein (e.g., human serum albumin (HSA), low-density lipoprotein (LDL), high-density lipoprotein (HDL), or globulin); an carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid, an oligonucleotide (e.g. an aptamer). Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

The conjugates can also include targeting groups, e.g., a cell or tissue targeting agent or group, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptide mimetic or an aptamer.

Targeting groups can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Targeting groups may also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, multivalent fucose, or aptamers.

The targeting group can be any ligand that is capable of targeting a specific receptor. Examples include, without limitation, folate, GalNAc, galactose, mannose, mannose-6P, apatamers, integrin receptor ligands, chemokine receptor ligands, transferrin, biotin, serotonin receptor ligands, PSMA, endothelin, GCPII, somatostatin, LDL, and HDL ligands. In particular embodiments, the targeting group is an aptamer. The aptamer can be unmodified or have any combination of modifications disclosed herein.

In still other embodiments, the GDA is covalently conjugated to a cell penetrating polypeptide. The cell-penetrating peptide may also include a signal sequence. The conjugates of the invention can be designed to have increased stability; increased cell transfection; and/or altered the biodistribution (e.g., targeted to specific tissues or cell types).

Conjugating moieties may be added to the GDA antibodies such that they allow labeling or flagging the GPC for clearance. Such tagging/flagging molecules include, but are not limited to ubiquitin, fluorescent molecules, human influenza hemaglutinin (HA), c-myc (a 10 amino acid segment of the human protooncogene myc with sequence EQKLISEEDL (SEQ ID NO: 317)), histidine (His), flag (a short peptide of sequence DYKDDDDK (SEQ ID NO: 318)), glutathione S-transferase (GST), V5 (a paramyxovirus of simian virus 5 epitope), biotin, avidin, streptavidin, horse radish peroxidase (HRP) and digoxigenin.

In some embodiments, GDAs may be combined with other GDAs, GPCs, GPC modulatory factors or other molecule in the treatment of a disease or condition. Such combinations may hasten or slow the release of a growth factor from a natural depot or from a GPC (including any GPC administered in combination with a GDA of the invention).

### Growth Factor Directed Agents: Antibodies, Manufacture

Antibodies of the present invention may be polyclonal or monoclonal or recombinant, produced by methods known in the art or as described in this application.

In some embodiments, the antibodies of the present invention may be labeled for purposes of detection with a detectable label known by one of skill in the art. The label can be a radioisotope, fluorescent compound, chemiluminescent compound, enzyme, or enzyme co-factor, or any other labels known in the art. In some aspects, the antibody that binds to a desired antigen is not labeled, but may be detected by binding of a labeled secondary antibody that specifically binds to the primary antibody.

Antibodies of the present invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), intracellularly made antibodies (i.e., intrabodies), and epitope-binding fragments of any of the above. Antibodies of the present invention can be from any animal origin including birds and mammals. Preferably, such antibodies are of human, murine (e.g., mouse and rat), donkey, sheep, rabbit, goat, guinea pig, camel, horse, or chicken origin. The antibodies of the present invention can be monospecific or multispecific (e.g., bispecific, trispecific, or of greater multispecificity). Multispecific antibodies can be specific for different epitopes of a peptide of the present invention, or can be specific for both a peptide of the present invention, and a heterologous epitope, such as a heterologous peptide or solid support material. (See, e.g., WO 93/17715; WO 92/08802; WO 91/00360; WO 92/05793; Tutt, A. et al., Trispecific F(ab)3 derivatives that use cooperative signaling via the TCR/CD3 complex and CD2 to activate and redirect resting cytotoxic T cells. J Immunol. 1991 Jul. 1; 147(1):60-9; U.S. Pat. Nos. 4,474,893; 4,714,681; 4,925,648; 5,573,920; 5,601,819; and Kostelny, S. A. et al., Formation of a bispecific antibody by the use of leucine zippers. J Immunol. 1992 Mar. 1; 148(5):1547-53). For example, the antibodies may be produced against a peptide containing repeated units of a peptide sequence of the present invention, or they may be produced against a peptide containing two or more peptide sequences of the present invention, or the combination thereof.

In some embodiments, antibodies can be prepared from any region of the proteins or peptides taught herein, for example prodomains or regions thereof. In addition, if a polypeptide is a receptor protein, e.g., a TGF-beta receptor, antibodies can be developed against (e.g., to target, bind or interact with) an entire receptor or portions of the receptor, for example, an intracellular domain, an extracellular domain, the entire transmembrane domain, specific transmembrane segments, any of the intracellular or extracellular loops, or any portions of these regions. Antibodies can also be developed against specific functional sites, such as the site of ligand binding, or sites that are glycosylated, phosphorylated, myristylated, or amidated, for example.

In the present invention, the peptides for generating antibodies preferably contain a sequence of at least 4, at least 5, at least 6, at least 7, more preferably at least 8, at least 9, at least 10, at least 11, at least 12, at least 13, at least 14, at least 15, and, preferably, between about 5 to about 50 amino acids in length, more preferably between about 10 to about 30 amino acids in length, even more preferably between about 10 to about 20 amino acids in length.

In the present invention, where larger polypeptides or proteins are used for generating antibodies, these preferably are at least 50, at least 55, at least 60, at least 70, at least 80, at least 90, or more amino acids in length.

Monoclonal antibodies of the present invention can be prepared using well-established methods known by those skilled in the art. In one embodiment, the monoclonal antibodies are prepared using hybridoma technology (Kohler, G. et al., Continuous cultures of fused cells secreting antibody of predefined specificity. Nature. 1975 Aug. 7; 256(5517):495-7). In a hybridoma method, a mouse, hamster, or other appropriate host animal, is typically immunized with an immunizing agent (e.g., a peptide of the invention) to elicit lymphocytes that produce or are capable of producing antibodies that will specifically bind to the immunizing agent. Alternatively, the lymphocytes may be immunized in vitro. The lymphocytes are then fused with an immortalized cell line using a suitable fusing agent, such as polyethylene glycol, to form a hybridoma cell (Goding, J. W., Monoclonal Antibodies: Principles and Practice. Academic Press. 1986; 59-1031). Immortalized cell lines are usually transformed mammalian cells, particularly myeloma cells of rodent, rabbit, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, D. et al., A human hybrid myeloma for production of human monoclonal antibodies. J Immunol. 1984 December; 133(6):3001-5; Brodeur, B. et al., Monoclonal Antibody Production Techniques and Applications. Marcel Dekker, Inc., New York. 1987; 33:51-63).

The culture medium in which the hybridoma cells are cultured can then be assayed for the presence of monoclonal antibodies. Preferably, the binding specificity (i.e., specific immunoreactivity) of monoclonal antibodies produced by the hybridoma cells is determined by immunoprecipitation or by an in vitro binding assay, such as radioimmunoassay (RIA) or enzyme-linked immunosorbent assay (ELISA). Such techniques and assays are known by those skilled in the art. The binding specificity of the monoclonal antibody can, for example, be determined by Scatchard analysis (Munson, P. J. et al., Ligand: a versatile computerized approach for characterization of ligand-binding systems. Anal Biochem. 1980 Sep. 1; 107(1):220-39).

After the desired hybridoma cells are identified, the clones may be subcloned by limiting dilution procedures and grown by standard methods. Suitable culture media for this purpose include, for example, Dulbecco's Modified Eagle's Medium or RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

In another embodiment, the monoclonal antibodies of the present invention can also be made by recombinant DNA methods, such as those described in U.S. Pat. No. 4,816,567, which is hereby incorporated by reference in its entirety. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

In another embodiment, antibodies of the present invention can also be produced by various procedures known by those skilled in the art. For the production of polyclonal antibodies in vivo, host animals, such as rabbits, rats, mice, sheep, or goats, are immunized with either free or carrier-coupled peptides, for example, by intraperitoneal and/or intradermal injection. Injection material is typically an emulsion containing about 100 μg of peptide or carrier protein. Various adjuvants can also be used to increase the immunological response, depending on the host species. Adjuvants include, but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art. Several booster injections may be needed, for instance, at intervals of about two weeks, to provide a useful titer of antibody which can be detected, for example, by ELISA assay using free peptide adsorbed to a solid surface. The titer of antibodies in serum from an immunized animal can be increased by selection of antibodies, e.g., by adsorption of the peptide onto a solid support and elution of the selected antibodies according to methods well known in the art.

GDAs comprising antibodies, variants and fragments thereof may be selected and produced using high throughput methods of discovery. In one embodiment, GDAs comprising synthetic antibodies, variants and fragments thereof are produced through the use of display libraries. The term “display” as used herein, refers to the expression or “display” of proteins or peptides on the surface of a given host. The term “library” as used herein, refers to a collection of unique cDNA sequences. A library may contain from as little as two unique cDNAs to hundreds of billions of unique cDNAs. In a preferred embodiment, GDAs comprising synthetic antibodies are produced using antibody display libraries or antibody fragment display libraries. The term “antibody fragment display library” as used herein, refers to a display library wherein each member encodes an antibody fragment containing at least one variable region of an antibody. Such antibody fragments are preferably Fab fragments, but other antibody fragments such as single-chain variable fragments (scFvs) are contemplated as well. In an Fab antibody fragment library, each Fab encoded may be identical except for the amino acid sequence contained within the variable loops of the complementarity determining regions (CDRs) of the Fab fragment. In an alternative or additional embodiment, amino acid sequences within the individual V_{H} and/or V_{L} regions may differ as well.

Display libraries may be expressed in a number of possible hosts including, but not limited to yeast, bacteriophage, bacteria and retroviruses. Additional display technologies that may be used include ribosome-display, microbead-display and protein-DNA linkage techniques. In a preferred embodiment, Fab display libraries are expressed in yeast or in bacteriophages (also referred to herein as “phages” or “phage particles”. When expressed, the Fabs decorate the surface of the phage or yeast where they can interact with a given antigen. An antigen comprising a GPC, growth factor, or an antigen from a desired target site may be used to select phage particles or yeast cells expressing antibody fragments with the highest affinity for that antigen. The DNA sequence encoding the CDR of the bound antibody fragment can then be determined through sequencing using the bound particle or cell. In one embodiment, positive selection is used in the development of antibodies. As used herein, the term “positive selection” refers to processes by which antibodies and/or fragments thereof are selected from display libraries based on affinity for antigens containing target sites. In some embodiments, negative selection is utilized in the development of antibodies. As used herein, the term “negative selection” refers to processes by which antigens that lack target sites for antibody production are used to exclude antibodies and/or fragments thereof from a given display library during antibody development. In some embodiments, both positive and negative selection are utilized during multiple rounds of selection in the development of antibodies using display libraries.

In yeast display, cDNA encoding different antibody fragments are introduced into yeast cells where they are expressed and the antibody fragments are “displayed” on the cell surface as described by Chao et al. (Chao, G. et al., Isolating and engineering human antibodies using yeast surface display. Nat Protoc. 2006; 1(2):755-68). In yeast surface display, expressed antibody fragments contain an additional domain comprising the yeast agglutinin protein, Aga2p. This domain allows the antibody fragment fusion protein to attach to the outer surface of the yeast cell through the formation of disulphide bonds with surface-expressed Aga1p. The result is a yeast cell, coated in a particular antibody fragment. Display libraries of cDNA encoding these antibody fragments are utilized initially in which the antibody fragments each have a unique sequence. These fusion proteins are expressed on the cell surface of millions of yeast cells where they can interact with a desired antigenic target peptide, incubated with the cells. Target peptides may be covalently or otherwise modified with a chemical or magnetic group to allow for efficient cell sorting after successful binding with a suitable antibody fragment takes place. Recovery may be by way of magnetic-activated cell sorting (MACS), fluorescence-activated cell sorting (FACS) or other cell sorting methods known in the art. Once a subpopulation of yeast cells is selected, the corresponding plasmids may be analyzed to determine the CDR sequence.

Bacteriophage display methods typically utilize filamentous phage including fd, F1 and M13 virions. Such strains are non-lytic, allowing for continued propagation of the host and increased viral titres. Examples of phage display methods that can be used to make the antibodies of the present invention include those disclosed in Miersch et al. (Miersch, S. et al., Synthetic antibodies: Concepts, potential and practical considerations. Methods. 2012 August; 57(4):486-98), Bradbury et al. (Bradbury, A. R. et al., Beyond natural antibodies: the power of in vitro display technologies. Nat Biotechnol. 2011 March; 29(3):245-54), Brinkman et al. (Brinkmann, U. et al., Phage display of disulfide-stabilized Fv fragments. J Immunol Methods. 1995 May 11; 182(1):41-50); Ames et al. (Ames, R. S. et al., Conversion of murine Fabs isolated from a combinatorial phage display library to full length immunoglobulins. J Immunol Methods. 1995 Aug. 18; 184(2):177-86); Kettleborough et al. (Kettleborough, C. A. et al., Isolation of tumor cell-specific single-chain Fv from immunized mice using phage-antibody libraries and the re-construction of whole antibodies from these antibody fragments. Eur J Immunol. 1994 April; 24(4):952-8); Persic et al. (Persic, L. et al., An integrated vector system for the eukaryotic expression of antibodies or their fragments after selection from phage display libraries. Gene. 1997 Mar. 10; 187(1):9-18.); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5, 969,108, each of which is incorporated herein by reference in its entirety. Antibody fragment expression on bacteriophages may be carried out by inserting the cDNA encoding the fragment into the gene expressing a viral coat protein. The viral coat of filamentous bacteriophages is made up of five coat proteins, encoded by a single-stranded genome. Coat protein pIII is the preferred protein for antibody fragment expression, typically at the N-terminus. If antibody fragment expression compromises the function of pIII, viral function may be restored through coexpression of a wild type pIII, although such expression will reduce the number of antibody fragments expressed on the viral coat, but may enhance access to the antibody fragment by the target antigen. Expression of viral as well as antibody fragment proteins may alternatively be encoded on multiple plasmids. This method may be used to reduce the overall size of infective plasmids and enhance the transformation efficiency.

As described above, after selection of a host expressing a high affinity antibody or antibody fragment, the coding regions from the antibody or antibody fragment can be isolated and used to generate whole antibodies, including human antibodies, or any other desired antigen binding fragment, and expressed in any desired host, including mammalian cells, insect cells, plant cells, yeast, and bacteria, e.g., as described in detail below.

The DNA sequence encoding a high affinity antibody can be mutated for additional rounds of selection in a process known as affinity maturation. The term “affinity maturation”, as used herein, refers to a method whereby antibodies are produced with increasing affinity for a given antigen through successive rounds of mutation and selection of antibody- or antibody fragment-encoding cDNA sequences. In a preferred embodiment, this process is carried out in vitro. To accomplish this, amplification of CDR coding sequences may be carried out using error-prone PCR to produce millions of copies containing mutations including, but not limited to point mutations, regional mutations, insertional mutations and deletional mutations. As used herein, the term “point mutation” refers to a nucleic acid mutation in which one nucleotide within a nucleotide sequence is changed to a different nucleotide. As used herein, the term “regional mutation” refers to a nucleic acid mutation in which two or more consecutive nucleotides are changed to different nucleotides. As used herein, the term “insertional mutation” refers to a nucleic acid mutation in which one or more nucleotides are inserted into a nucleotide sequence. As used herein, the term “deletional mutation” refers to a nucleic acid mutation in which one or more nucleotides are removed from a nucleotide sequence. Insertional or deletional mutations may include the complete replacement of an entire codon or the change of one codon to another by altering one or two nucleotides of the starting codon.

Mutagenesis may be carried out on CDR-encoding cDNA sequences to create millions of mutants with singular mutations in CDR heavy and light chain regions. In another approach, random mutations are introduced only at CDR residues most likely to improve affinity. These newly generated mutagenic libraries can be used to repeat the process to screen for clones that encode antibody fragments with even higher affinity for the target peptide. Continued rounds of mutation and selection promote the synthesis of clones with greater and greater affinity (Chao, G. et al., Isolating and engineering human antibodies using yeast surface display. Nat Protoc. 2006; 1(2):755-68).

Examples of techniques that can be used to produce antibodies and antibody fragments, such as Fabs and scFvs, include those described in U.S. Pat. Nos. 4,946,778 and 5,258,498; Miersch et al. (Miersch, S. et al., Synthetic antibodies: Concepts, potential and practical considerations. Methods. 2012 August; 57(4):486-98), Chao et al. (Chao, G. et al., Isolating and engineering human antibodies using yeast surface display. Nat Protoc. 2006; 1(2):755-68), Huston et al. (Huston, J. S. et al., Protein engineering of single-chain Fv analogs and fusion proteins. Methods Enzymol. 1991; 203:46-88); Shu et al. (Shu, L. et al., Secretion of a single-gene-encoded immunoglobulin from myeloma cells. Proc Natl Acad Sci USA. 1993 Sep. 1; 90(17):7995-9); and Skerra et al. (Skerra, A. et al., Assembly of a functional immunoglobulin Fv fragment in Escherichia coli. Science. 1988 May 20; 240(4855):1038-41), each of which is incorporated herein by reference in its entirety.

For some uses, including the in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal immunoglobulin and a human immunoglobulin constant region. Methods for producing chimeric antibodies are known in the art. (Morrison, S. L., Transfectomas provide novel chimeric antibodies. Science. 1985 Sep. 20; 229(4719):1202-7; Gillies, S. D. et al., High-level expression of chimeric antibodies using adapted cDNA variable region cassettes. J Immunol Methods. 1989 Dec. 20; 125(1-2):191-202.; and U.S. Pat. Nos. 5,807,715; 4,816,567; and 4,816,397, which are incorporated herein by reference in their entirety). Humanized antibodies are antibody molecules from non-human species that bind to the desired antigen and have one or more complementarity determining regions (CDRs) from the nonhuman species and framework regions from a human immunoglobulin molecule. Often, framework residues in the human framework regions are substituted with corresponding residues from the CDR and framework regions of the donor antibody to alter, preferably improve, antigen binding. These framework substitutions are identified by methods well known in the art, e.g., by modeling of the interactions of the CDR and framework residues to identify framework residues important for antigen binding, and by sequence comparison to identify unusual framework residues at particular positions. (U.S. Pat. Nos. 5,693,762 and 5,585,089; Riechmann, L. et al., Reshaping human antibodies for therapy. Nature. 1988 Mar. 24; 332(6162):323-7, which are incorporated herein by reference in their entireties). Antibodies can be humanized using a variety of techniques known in the art, including, for example, CDR-grafting (EP 239,400; PCT publication WO 91/09967; U.S. Pat. Nos. 5,225,539; 5,530,101; and 5,585,089); veneering or resurfacing (EP 592,106; EP 519,596; Padlan, E. A., A possible procedure for reducing the immunogenicity of antibody variable domains while preserving their ligand-binding properties. Mol Immunol. 1991 April-May; 28(4-5):489-98; Studnicka, G. M. et al., Human-engineered monoclonal antibodies retain full specific binding activity by preserving non-CDR complementarity-modulating residues. Protein Eng. 1994 June; 7(6):805-14; Roguska, M. A. et al., Humanization of murine monoclonal antibodies through variable domain resurfacing. Proc Natl Acad Sci USA. 1994 Feb. 1; 91(3):969-73); and chain shuffling (U.S. Pat. No. 5,565,332); each of which is incorporated herein by reference in their entirety.

Completely human antibodies are particularly desirable for therapeutic treatment of human patients, so as to avoid or alleviate immune reaction to foreign protein. Human antibodies can be made by a variety of methods known in the art, including the antibody display methods described above, using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin polynucleotides. For example, the human heavy and light chain immunoglobulin polynucleotide complexes can be introduced randomly, or by homologous recombination, into mouse embryonic stem cells. Alternatively, the human variable region, constant region, and diversity region may be introduced into mouse embryonic stem cells, in addition to the human heavy and light chain polynucleotides. The mouse heavy and light chain immunoglobulin polynucleotides can be rendered nonfunctional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention.

Thus, using such a technique, it is possible to produce useful human IgG, IgA, IgM, IgD and IgE antibodies. For an overview of the technology for producing human antibodies, see Lonberg and Huszar (Lonberg, N. et al., Human antibodies from transgenic mice. Int Rev Immunol. 1995; 13(1):65-93). For a detailed discussion of the technology for producing human antibodies and human monoclonal antibodies and protocols for producing such antibodies, see, e.g., PCT publications WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; 5,939,598; 6,075,181; and 6,114,598, each of which are incorporated by reference herein in their entirety. In addition, companies such as Abgenix, Inc. (Fremont, Calif.), Protein Design Labs, Inc. (Mountain View, Calif.) and Genpharm (San Jose, Calif.) can be engaged to provide human antibodies directed against a selected antigen using technology similar to the above described technologies.

Once an antibody molecule of the present invention has been produced by an animal, a cell line, chemically synthesized, or recombinantly expressed, it can be purified (i.e., isolated) by any method known in the art for the purification of an immunoglobulin or polypeptide molecule, for example, by chromatography (e.g., ion exchange, affinity, particularly by affinity for the specific antigen, Protein A, and sizing column chromatography), centrifugation, differential solubility, or by any other standard technique for the purification of proteins. In addition, the antibodies of the present invention or fragments thereof can be fused to heterologous polypeptide sequences described herein or otherwise known in the art, to facilitate purification.

### Nucleic Acids

The present invention embraces nucleic acid molecules. In some embodiments, nucleic acids encode GDAs. Such nucleic acid molecules include, without limitation, DNA molecules, RNA molecules, polynucleotides, oligonucleotides, mRNA molecules, vectors, plasmids and the like. In some embodiments, nucleic acids are themselves GDAs. Such GDAs may comprise nucleic acid aptamers. Also included are cells programmed or generated to express the nucleic acid molecules disclosed above.

### II. Targets of the Invention

Broadly, any biomolecule, cellular structure, cellular signaling pathway or cell niche capable of being bound, sequestered, contacted or altered by a GDA composition of the invention is considered a “target” of the invention. Targets of the present invention, when referring to the binding or interaction with GDAs such as antibodies include ECCM signaling molecules, specifically those of the TGF-beta and integrin families.

### The TGF-Beta Family

The TGF-beta family is of wide importance. In embryogenesis, its 33 members regulate all major developmental processes and the details of the formation of almost all organs. Although much of the key work regulated by this family is accomplished by the time of birth, afterwards the family continues to regulate many processes including immune responses, wound healing, bone growth, endocrine functions, and muscle mass.

In some cytokine families, restricted expression of the ligands or the receptors is key to regulating signaling. However, many TGF-beta superfamily ligands and receptors are expressed widely, and release of the ligand from a latent form stored extracellularly is the key activating step. The importance of the ligands is emphasized by their expansion in the evolution of TGF-beta signaling pathway components. TGF-beta, alone, has been implicated in fibrosis, and is a major target in kidney fibrosis, pulmonary fibrosis and myelofibrosis, each of which is recognized as a major medical unmet need.

The 33 members of the TGF-beta superfamily, as well as the four ligands related to glial cell derived neurotrophic factor (GDNF) are listed in Table 3.

TABLE 3
TGF-beta Superfamily Targets

| | | | SEQ |
|---|---|---|---|
| | | Accession | ID |
| Name | Symbol | Number | NO |
| Transforming growth factor beta 1 | TGFB1 | NP_000651.3 | 74 |
| Transforming growth factor beta 2, | TGFB2 | NP_001129071.1 | 75 |
| isoform 1 | | | |
| Transforming growth factor beta 2, | TGFB2 | NP_003229.1 | 76 |
| isoform 2 | | | |
| Transforming growth factor beta 3 | TGFB3 | NP_003230.1 | 77 |
| Growth/differentiation factor 11 | GDF11 | NP_005802.1 | 78 |
| Growth/differentiation factor 8 | GDF8 | NP_005250.1 | 79 |
| Inhibin beta A chain | INHBA | NP_002183.1 | 80 |
| Inhibin beta B chain | INHBB | NP_002184.2 | 81 |
| Inhibin beta C chain | INHBC | NP_005529.1 | 82 |
| Inhibin beta E chain | INHBE | NP_113667.1 | 83 |
| Left-right determination factor 1 | LEFTY1 | NP_066277.1 | 84 |
| Left-right determination factor 2 | LEFTY2 | NP_003231.2 | 85 |
| Growth/differentiation factor 15 | GDF15 | NP_004855.2 | 86 |
| Anti-Mullerian hormone | AMH | NP_000470.2 | 87 |
| Inhibin alpha | INHA | NP_002182.1 | 88 |
| Growth/differentiation factor 1 | GDF1 | NP_001483.3 | 89 |
| Growth/differentiation factor 3 | GDF3 | NP_065685.1 | 90 |
| Growth/differentiation factor 5 | GDF5 | NP_000548.1 | 91 |
| Growth/differentiation factor 6 | GDF6 | NP_001001557.1 | 92 |
| Growth/differentiation factor 7 | GDF7 | NP_878248.2 | 93 |
| Bone morphogenetic protein 10 | BMP10 | NP_055297.1 | 94 |
| Growth/differentiation factor 2 | GDF2 | NP_057288.1 | 95 |
| Nodal homolog (mouse) | NODAL | NP_060525.3 | 96 |
| Bone morphogenetic protein 2 | BMP2 | NP_001191.1 | 97 |
| Bone morphogenetic protein 4 | BMP4 | NP_001193.2 | 98 |
| Bone morphogenetic protein 5 | BMP5 | NP_066551.1 | 99 |
| Bone morphogenetic protein 6 | BMP6 | NP_001709.1 | 100 |
| Bone morphogenetic protein 7 | BMP7 | NP_001710.1 | 101 |
| Bone morphogenetic protein 8A | BMP8A | NP_861525.2 | 102 |
| Bone morphogenetic protein 8B | BMP8B | NP_001711.2 | 103 |
| Bone morphogenetic protein 15 | BMP15 | NP_005439.2 | 104 |
| Growth/differentiation factor 9 | GDF9 | NP_005251.1 | 105 |
| Bone morphogenetic protein 3 | BMP3 | NP_001192.2 | 106 |
| Growth/differentiation factor 10 | GDF10 | NP_004953.1 | 107 |
| Glial cell line-derived neurotrophic | GDNF | NP_000505.1 | 108 |
| factor | | | |
| Neurturin | NRTN | NP_004549.1 | 109 |
| Persephin | PSPN | NP_004149.1 | 110 |
| Artemin | ARTN | NP_476432.2 | 111 |

In one embodiment, GDA antibodies are designed to modulate myostatin activity. Myostatin is involved in regulating the degradation of damaged muscle fibrils, and its deficiency increases muscle mass (Rodino-Klapac, L. R. et al., Inhibition of myostatin with emphasis on follistatin as a therapy for muscle disease. Muscle Nerve. 2009 March; 39(3):283-96). In some embodiments, GDA antibodies serve to trigger degradation of the myostatin-prodomain complex and/or prevent activation of myostatin. In a further embodiment, GDA antibodies block the protease cleavage site recognized by members of the BMP1/tolloid protease family. Such antibodies to latent TGF-beta or latent myostatin might also be used to trigger degradation of latent TGF-beta-LTBP complexes, or latent myostatin complexes with proteoglycans after secretion by cells and prior to their deposition in the extracellular matrix. Such GDAs that inhibit myostatin activity may be used to repair and/or enhance muscle tissues.

### Integrins

Integrins are cell surface heterodimers formed by alpha and beta subunits, each of which has a transmembrane domain and in the N-terminal portion of the extracellular domain come together to form the ligand binding site. Thus, both the αᵥ and β₆ subunits form the ligand binding site of αᵥβ₆ integrin.

Like integrins αIIbβ₃, α₅β₁, and α₈β₁, αᵥ integrins recognize the Arg-Gly-Asp or RGD sequence as a key component of the ligand. However, αᵥβ₆ and αᵥβ₈ recognize more than just the RGD in TGF-beta, and are also quite unusual in binding with high affinity to TGF-beta1 and 3 without any need for activation. Essentially all other integrins require activation signals, provided by association with the actin cytoskeleton, for “inside-out” signals that induce a conformational change in the integrin ectodomain and increase affinity for ligand by about 1,000 to 10,000-fold. Knockouts of the β₆ and β₈ subunits have shown they are very important in pulmonary fibrosis and tolerance induction by dendritic cells, respectively.

Furthermore, the key role of the RGD motif and hence activation by αᵥβ₆ and αᵥβ₈ is shown by the similarity in the phenotypes of RGD/RGE TGF-beta1 knock-in and 136/138 and TGF-beta1 knockouts. The β₆ and β₈ (and β₅) subunits only associate with αᵥ.

A subset of half of integrin-α subunits contain an inserted, or αI, domain, which is the ligand-binding domain when present. Such integrins include the β₂ integrins selectively expressed on leukocytes such as α_{L}β₂, and the collagen-binding integrins α₁β₁, α₂β₁, α₁₀β₁, and α₁₁β₁. As its name very late activation antigen-1 (VLA-1) implies, α₁β₁ is expressed on lymphocytes only late (over a time course of days) after activation by specific antigen. Among all four collagen binding integrins, antibodies to α₁ have by far the most profound ability to inhibit in vivo models of disease in animals.

The crystal structures of many integrins have been determined. These include complexes of the integrin α₄β₇ bound to small molecule antagonists and the therapeutic antibodies Natalizumab and Vedolizumab (Yu, Y. et al. Structural specializations of α(4)β(7), an integrin that mediates rolling adhesion. J Cell Biol. 2012 Jan. 9; 196(1):131-46).

None of the antibodies in the art, however, bind to the ligand binding site which is defined by the small molecule antagonist in a cleft at the interface between the α4 and 137 subunits. Instead, the antibodies bind to epitopes that contain human/rodent amino acid substitutions, with the epitopes sufficiently close to the ligand binding site for the Fab fragment of the antibody to sterically interfere with one of the domains of the ligand, This domain is often not domain 1 that binds to the integrin but domain 2 which is nearby. Similarly, an antibody to the I domain of LFA 1 that was in the clinic for several years for the treatment of psoriasis, did not block binding directly. Instead, it sterically prevented domain 2 of ICAM-1 from coming close enough to LFA-1 to enable domain 1 of ICAM-1 to bind. The history of antibody therapeutic development to integrins is thus limited to particular human/rodent amino acid substitutions that enabled antibodies to be elicited, and secondarily to the functional selection of antibodies that also had the properties of blocking ligand binding.

In one embodiment, antibodies are contemplated which function specifically to bind or interact with the ligand binding site or other specific locations on the GPC.

Integrins are less conserved than TGF-beta family members. Among the collagen-binding integrin α-subunits, α₁ is 38, 34, and 37% identical to α₂, α₁₀, and α₁₁, respectively. However, conservation in the ligand-binding αI domain is higher, at 54-56%. Between human and mouse, the complete α₁ ectodomain and ligand-binding αI domain are 88% and 94% identical, respectively. The αᵥ subunit is 36 to 46% identical to other RGD-binding integrin α-subunits, and 19-25% identical to the α-subunits of other integrin subfamilies. Between human and mouse, the αᵥ and β₆ ectodomains are 93 and 90% identical, respectively.

Recently VLA-1 has also been identified as a receptor for semaphorin 7A. A subset of semaphorins, which mediate axon guidance and synapse formation in the nervous system, function in the immune system, and are also targets of the invention as blocking antibodies.

Integrins α₄β₁, α₄β₇, and αLβ₂ (LFA-1) are already proven targets for lymphocyte-mediated autoimmune disease including multiple sclerosis, Crohn's disease, and psoriasis. Integrins α₁β₁ (VLA-1) and the αᵥ integrins, αᵥβ₆ and αᵥβ₈, are promising targets for fibrosis and antibodies directed to each of these is contemplated by the present invention.

The integrin subunit targets of the present invention are listed in Table 4.

TABLE 4
Integrin Subunit Targets

| | | Accession | SEQ |
|---|---|---|---|
| Name | Gene Symbol | Number | ID NO |
| α₁ | ITGA1 | NP_852478.1 | 112 |
| α₂ | ITGA2 | NP_002194.2 | 113 |
| αIIb | ITGA2B | NP_000410.2 | 114 |
| α₃ | ITGA3 | NP_005492.1 | 115 |
| α₄ | ITGA4 | NP_000876.3 | 116 |
| α₅ | ITGA5 | NP_002196.2 | 117 |
| α₆ | ITGA6 | NP_001073286.1 | 118 |
| α₇ | ITGA7 | NP_001138468.1 | 119 |
| α₈ | ITGA8 | NP_003629.1 | 120 |
| α₉ | ITGA9 | NP_002198.2 | 121 |
| α₁₀ | ITGA10 | NP_003628.2 | 122 |
| α₁₁ | ITGA11 | NP_001004439.1 | 123 |
| α_{D} | ITGAD | NP_005344.2 | 124 |
| α_{E} | ITGAE | NP_002199.3 | 125 |
| α_{L} | ITGAL | NP_002200.2 | 126 |
| α_{M} | ITGAM | NP_001139280.1 | 127 |
| α_{V} | ITGAV | NP_002201.1 | 128 |
| α_{X} | ITGAX | NP_000878.2 | 129 |
| β₁ | ITGB1 | NP_391988.1 | 130 |
| β₂ | ITGB2 | NP_000202.2 | 131 |
| β₃ | ITGB3 | NP_000203.2 | 132 |
| β₄ | ITGB4 | NP_000204.3 | 133 |
| β₅ | ITGB5 | NP_002204.2 | 134 |
| β₆ | ITGB6 | NP_000879.2 | 135 |
| β₇ | ITGB7 | NP_000880.1 | 136 |
| β₈ | ITGB8 | NP_002205.1 | 137 |

### Type I, Type II and Type III Receptors

Type I, II and III receptors are also contemplated as targets of the present invention. Outside of the cell, TGF-beta can bind to either the type III TGF-beta receptor (RIII) or the type II TGF-beta receptor (RII). RIII merely functions in the presentation of TGF-beta to the RII receptor. RIII proteins are the most abundant and are important factors in determining overall TGF-beta signaling activity. They bind TGF-beta with high affinity and there are different types expressed in different cell types. TGF-beta receptor 3 (TGFBR3), also known as betaglycan, is ubiquitously expressed, while another RIII, endoglin, is primarily expressed in vascular endothelial cells. Both are type I transmembrane proteins with small intracellular domains and large extracellular domains. They are also both susceptible to cleavage of their extracellular domains, which become soluble antagonists, binding and neutralizing TGF-beta before it can interact with the cell.

Binding of TGF-beta to an RII leads to the recruitment of the type I TGF-beta receptor (RI). There are seven RI proteins also known as activin-like receptor kinases (ALKs) as well multiple RII proteins (listed in Table 5). Together, TGF-beta, RI and RII combine to form a growth factor-receptor complex (GRC). GRC formation stimulates the phosphorylation of RI on tyrosine and serine/threonine residues by an RI protein kinase. Once phosphorylated, RI itself exhibits kinase activity, leading to the phosphorylation and activation of Smad transcription factors.

Three groups of Smads exist including R-Smads (receptor-associated Smads), Co-Smads (co-operating Smads) and I-Smads (inhibitory Smads). R-Smads are transcription factors that remain inactive in the cytoplasm prior to activation by RI phosphorylation. Among the R-Smads are Smad1, Smad2, Smad3, Smad5 and Smad8. Phosphorylated R-Smads bind to the Co-Smad, Smad4, before traveling together to the nucleus to collaborate with other transcription factors in the expression of TGF-beta-responsive genes. Among the genes expressed are those of the I-Smads, Smad 6 and Smad7. Their expression is part of a negative-feedback loop to suppress continued TGF-beta signaling (Santibanez, J. F. et al., TGF-beta/TGF-beta receptor system and its role in physiological and pathological conditions. Clin Sci (Loud). 2011 September; 121(6):233-51; Blobe, G. C. et al., Role of transforming growth factor beta in human disease. N Engl J Med. 2000 May 4; 342(18):1350-8).

TABLE 5
Type I, II and III Receptor Targets

| | | Accession | SEQ |
|---|---|---|---|
| Name | Symbol | Number | ID NO |
| Type I Receptors | | | |
| Activin receptor-like kinase 1 | ALK1 | NP_001070869.1 | 138 |
| Activin receptor-like kinase 2 | ALK2 | NP_001104537.1 | 139 |
| Activin receptor-like kinase 3 | ALK3 | NP_004320.2 | 140 |
| Activin receptor-like kinase 4 | ALK4 | NP_064733.3 | 141 |
| Activin receptor-like kinase 5 | ALK5 | NP_004603.1 | 142 |
| Activin receptor-like kinase 6 | ALK6 | NP_001243722.1 | 143 |
| Activin receptor-like kinase 7 | ALK7 | NP_660302.2 | 144 |
| Type II Receptors | | | |
| TGF-beta receptor 2 | TGFBR2 | NP_001020018.1 | 145 |
| BMP receptor 2 | BMPR2 | NP_001195.2 | 146 |
| Type III Receptors | | | |
| TGF-beta receptor 3 | TGFBR3 | NP_003234.2 | 147 |
| Endoglin | ENG | NP_001108225.1 | 148 |

According to the present invention, antibodies may be designed which selectively block only the RI, RII or RIII receptor binding sites, and as such these antibodies would act as antagonists and function superior to antibodies that block binding to both sites. Antibodies specific for a single receptor binding site are also contemplated which may be used analogously to receptor-Fc fusion proteins as antagonists, which are currently in clinical trials and show efficacy. Additionally, antibodies that bind to sites of interaction between the receptor types to enhance or disrupt GRC formation are contemplated.

### Notch and Wnt Pathway Members

The proteins of the notch and wnt signaling pathways are over 90% identical between mouse and human. Notch 1 is mutated in 50% of acute lymphocytic leukemia and the mutations are activating. Canonical Notch signaling involves the binding of a ligand protein to a Notch transmembrane receptor. Binding initiates proteolytic cleavage, releasing the intracellular domain where it can travel to the nucleus and participate in Notch-dependent gene regulation (Andersson, E. R. et al., Notch signaling: simplicity in design, versatility in function. Development. 2011 September; 138(17):3593-612). In some embodiments, Notch transmembrane receptors comprise regulatory elements that modulate proteolytic cleavage of the receptor. Some progress has been made in the development of Notch antibodies. “Wnt” proteins, as referred to herein, are a class of cell signaling proteins known to direct cell polarity, plasticity, growth and proliferation. In some embodiments, Wnt proteins may be growth factors. They are named through a combination of the genes Wingless, identified in flies, and the Int-1 gene, identified by its upregulation in virally induced breast tumors. Canonical Wnt signaling involves the binding of a Wnt protein to a corresponding Frizzled (Fz) receptor and the coreceptor, low density lipoprotein receptor-like protein (LRP) 5 or 6. The intracellular effect is the rescue of beta-catenin from degradation, allowing it to travel to the nucleus and participate in genetic regulation. Wnt signaling has been shown to be disrupted in some diseases. Examples of such diseases include, but are not limited to cancer, diabetes and coronary artery disease (Clevers, H. et al., Wnt/β-catenin signaling and disease. Cell. 2012 Jun. 8; 149(6):1192-205). In some embodiments, Wnt proteins comprise regulatory elements. Such elements may be required for associations of Wnt with other factors. In one embodiment, Wnt regulatory elements modulate the interaction of Wnt with ECCM components including, but not limited to sugar moieties and/or proteoglycans. In some embodiments, GDAs may target regulatory elements on Notch and/or Wnt resulting in stimulation, enhancement, inhibition and/or blockage of Notch and/or Wnt activity. In some embodiments, GDAs may act to modulate Notch or Wnt activity by targeting proteins involved in Wnt and Notch-dependent cell signaling. Such targets are listed in Table 6.

TABLE 6
Notch and Wnt Targets

| | | Accession | SEQ ID |
|---|---|---|---|
| Name | Symbol | Number | NO |
| ADAM metallopeptidase domain 10 | ADAM10 | NP_001101.1 | 149 |
| ADAM metallopeptidase domain 17 | ADAM17 | NP_003174.3 | 150 |
| Adenomatous polyposis coli | APC | NP_001120982.1 | 151 |
| Amino-terminal enhancer of split | AES | NP_945320.1 | 152 |
| Axin 1 | AXIN1 | NP_003493.1 | 153 |
| Axin 2 | AXIN2 | NP_004646.3 | 154 |
| B-cell CLL/lymphoma 9 | BCL9 | NP_004317.2 | 155 |
| Beta-transducin repeat containing | BTRC | NP_378663.1 | 156 |
| Cas-Br-M (murine) ecotropic retroviral | CBL | NP_005179.2 | 157 |
| transforming sequence | | | |
| Casein kinase 1, alpha 1 | CSNK1A1 | NP_001020276.1 | 158 |
| Casein kinase 2, alpha 1 polypeptide | CSNK2A1 | NP_808227.1 | 159 |
| CASP8 and FADD-like apoptosis regulator | CFLAR | NP_001120655.1 | 160 |
| Catenin (cadherin-associated protein), beta | CTNNB1 | NP_001091679.1 | 161 |
| 1, 88 kDa | | | |
| Catenin, beta interacting protein 1 | CTNNBIP1 | NP_001012329.1 | 162 |
| CD44 molecule (Indian blood group) | CD44 | NP_000601.3 | 163 |
| C-fos induced growth factor (vascular | FIGF | NP_004460.1 | 164 |
| endothelial growth factor D) | | | |
| Conserved helix-loop-helix ubiquitous | CHUK | NP_001269.3 | 165 |
| kinase | | | |
| C-terminal binding protein 1 | CTBP1 | NP_001319.1 | 166 |
| CXXC finger protein 4 | CXXC4 | NP_079488.2 | 167 |
| Cyclin D1 | CCND1 | NP_444284.1 | 168 |
| Cyclin D2 | CCND2 | NP_001750.1 | 169 |
| Cyclin E1 | CCNE1 | NP_001229.1 | 170 |
| Cyclin-dependent kinase inhibitor 1A (p21, | CDKN1A | NP_001207707.1 | 171 |
| Cip1) | | | |
| Delta-like 1 (Drosophila) | DLL1 | NP_005609.3 | 172 |
| Delta-like 3 (Drosophila) | DLL3 | NP_058637.1 | 173 |
| Delta-like 4 (Drosophila) | DLL4 | NP_061947.1 | 174 |
| Deltex homolog 1 (Drosophila) | DTX1 | NP_004407.2 | 175 |
| Dickkopf homolog 1 (Xenopus laevis) | DKK1 | NP_036374.1 | 176 |
| Dickkopf homolog 3 (Xenopus laevis) | DKK3 | NP_001018067.1 | 177 |
| Disabled homolog 2, mitogen-responsive | DAB2 | NP_001334.2 | 178 |
| phosphoprotein (Drosophila) | | | |
| Dishevelled associated activator of | DAAM1 | NP_055807.1 | 179 |
| morphogenesis 1 | | | |
| Dishevelled, dsh homolog 1 (Drosophila) | DVL1 | NP_004412.2 | 180 |
| Dishevelled, dsh homolog 2 (Drosophila) | DVL2 | NP_004413.1 | 181 |
| DIX domain containing 1 | DIXDC1 | NP_001033043.1 | 182 |
| E1A binding protein p300 | EP300 | NP_001420.2 | 183 |
| FBJ murine osteosarcoma viral oncogene | FOS | NP_005243.1 | 184 |
| homolog | | | |
| F-box and WD repeat domain containing | FBXW11 | NP_036432.2 | 185 |
| 11 | | | |
| F-box and WD repeat domain containing 4 | FBXW4 | NP_071322.1 | 186 |
| Fibroblast growth factor 4 | FGF4 | NP_001998.1 | 187 |
| Forkhead box N1 | FOXN1 | NP_003584.2 | 188 |
| FOS-like antigen 1 | FOSL1 | NP_005429.1 | 189 |
| Frequently rearranged in advanced T-cell | FRAT1 | NP_005470.2 | 190 |
| lymphomas | | | |
| Frizzled family receptor 1 | FZD1 | NP_003496.1 | 191 |
| Frizzled family receptor 2 | FZD2 | NP_001457.1 | 192 |
| Frizzled family receptor 3 | FZD3 | NP_059108.1 | 193 |
| Frizzled family receptor 4 | FZD4 | NP_036325.2 | 194 |
| Frizzled family receptor 5 | FZD5 | NP_003459.2 | 195 |
| Frizzled family receptor 6 | FZD6 | NP_003497.2 | 196 |
| Frizzled family receptor 7 | FZD7 | NP_003498.1 | 197 |
| Frizzled family receptor 8 | FZD8 | NP_114072.1 | 198 |
| Frizzled family receptor 9 | FZD9 | NP_003499.1 | 199 |
| Frizzled-related protein | FRZB | NP_001454.2 | 200 |
| GLI family zinc finger 1 | GLI1 | NP_005260.1 | 201 |
| Glycogen synthase kinase 3 alpha | GSK3A | NP_063937.2 | 202 |
| Glycogen synthase kinase 3 beta | GSK3B | NP_002084.2 | 203 |
| Hairless homolog (mouse) | HR | NP_005135.2 | 204 |
| Hairy and enhancer of split 1, (Drosophila) | HES1 | NP_005515.1 | 205 |
| Hairy and enhancer of split 5 (Drosophila) | HES5 | NP_001010926.1 | 206 |
| Hairy/enhancer-of-split related with | HEY1 | NP_001035798.1 | 207 |
| YRPW (SEQ ID NO: 319) motif 1 | | | |
| Hairy/enhancer-of-split related with | HEY2 | NP_036391.1 | 208 |
| YRPW (SEQ ID NO: 319) motif 2 | | | |
| Hairy/enhancer-of-split related with | HEYL | NP_055386.1 | 209 |
| YRPW (SEQ ID NO: 319) motif-like | | | |
| Histone deacetylase 1 | HDAC1 | NP_004955.2 | 210 |
| Homeobox B4 | HOXB4 | NP_076920.1 | 211 |
| Inhibitor of DNA binding 1, dominant | ID1 | NP_002156.2 | 212 |
| negative helix-loop-helix protein | | | |
| Interferon, gamma | IFNG | NP_000610.2 | 213 |
| Interleukin 17B | IL17B | NP_055258.1 | 214 |
| Interleukin 2 receptor, alpha | IL2RA | NP_000408.1 | 215 |
| Jagged 1 | JAG1 | NP_000205.1 | 216 |
| Jagged 2 | JAG2 | NP_002217.3 | 217 |
| Jun proto-oncogene | JUN | NP_002219.1 | 218 |
| Keratin 1 | KRT1 | NP_006112.3 | 219 |
| Kringle containing transmembrane protein 1 | KREMEN1 | NP_114434.3 | 220 |
| LFNG O-fucosylpeptide 3-beta-N- | LFNG | NP_001035257.1 | 221 |
| acetylglucosaminyltransferase | | | |
| LIM domain only 2 (rhombotin-like 1) | LMO2 | NP_005565.2 | 222 |
| Loricrin | LOR | NP_000418.2 | 223 |
| Low density lipoprotein receptor-related | LRP5 | NP_002326.2 | 224 |
| protein 5 | | | |
| Low density lipoprotein receptor-related | LRP6 | NP_002327.2 | 225 |
| protein 6 | | | |
| Lymphoid enhancer-binding factor 1 | LEF1 | NP_057353.1 | 226 |
| Mastermind-like 1 (Drosophila) | MAML1 | NP_055572.1 | 227 |
| Mastermind-like 2 (Drosophila) | MAML2 | NP_115803.1 | 228 |
| Matrix metallopeptidase 7 (matrilysin, | MMP7 | NP_002414.1 | 229 |
| uterine) | | | |
| MFNG O-fucosylpeptide 3-beta-N- | MFNG | NP_002396.2 | 230 |
| acetylglucosaminyltransferase | | | |
| Mitogen-activated protein kinase 8 | MAPK8 | NP_620637.1 | 231 |
| Naked cuticle homolog 1 (Drosophila) | NKD1 | NP_149110.1 | 232 |
| Nemo-like kinase | NLK | NP_057315.3 | 233 |
| Neuralized homolog (Drosophila) | NEURL | NP_004201.3 | 234 |
| Nicastrin | NCSTN | NP_056146.1 | 235 |
| Notch 1 | NOTCH1 | NP_060087.3 | 236 |
| Notch 2 | NOTCH2 | NP_077719.2 | 237 |
| Notch 2 N-terminal like | NOTCH2NL | NP_982283.2 | 238 |
| Notch 3 | NOTCH3 | NP_000426.2 | 239 |
| Notch 4 | NOTCH4 | NP_004548.3 | 240 |
| Nuclear factor of activated T-cells, | NFATC1 | NP_765975.1 | 241 |
| cytoplasmic, calcineurin-dependent 1 | | | |
| Nuclear factor of kappa light polypeptide | NFKB1 | NP_003989.2 | 242 |
| gene enhancer in B-cells 1 | | | |
| Nuclear factor of kappa light polypeptide | NFKB2 | NP_001070962.1 | 243 |
| gene enhancer in B-cells 2 (p49/p100) | | | |
| Nuclear receptor corepressor 2 | NCOR2 | NP_006303.4 | 244 |
| Nuclear receptor subfamily 4, group A, | NR4A2 | NP_006177.1 | 245 |
| member 2 | | | |
| Numb homolog (Drosophila) | NUMB | NP_001005743.1 | 246 |
| Paired box 5 | PAX5 | NP_057953.1 | 247 |
| Paired-like homeodomain 2 | PITX2 | NP_001191326.1 | 248 |
| Peroxisome proliferator-activated receptor | PPARD | NP_001165289.1 | 249 |
| delta | | | |
| Peroxisome proliferator-activated receptor | PPARG | NP_056953.2 | 250 |
| gamma | | | |
| Porcupine homolog (Drosophila) | PORCN | NP_982301.1 | 251 |
| Pre T-cell antigen receptor alpha | PTCRA | NP_001230097.1 | 252 |
| Presenilin 1 | PSEN1 | NP_000012.1 | 253 |
| Presenilin 2 (Alzheimer disease 4) | PSEN2 | NP_000438.2 | 254 |
| Presenilin enhancer 2 homolog (C. elegans) | PSENEN | NP_758844.1 | 255 |
| Prickle homolog 1 (Drosophila) | PRICKLE1 | NP_001138355.1 | 256 |
| Protein O-fucosyltransferase 1 | POFUT1 | NP_056167.1 | 257 |
| Pygopus homolog 1 (Drosophila) | PYGO1 | NP_056432.1 | 258 |
| Ras homolog gene family, member A | RHOA | NP_001655.1 | 259 |
| Ras homolog gene family, member U | RHOU | NP_067028.1 | 260 |
| Recombination signal binding protein for | RBPJL | NP_055091.2 | 261 |
| immunoglobulin kappa J region-like | | | |
| RFNG O-fucosylpeptide 3-beta-N- | RFNG | NP_002908.1 | 262 |
| acetylglucosaminyltransferase | | | |
| Runt-related transcription factor 1 | RUNX1 | NP_001745.2 | 263 |
| RuvB-like 1 (E. coli) | RUVBL1 | NP_003698.1 | 264 |
| SCL/TAL1 interrupting locus | STIL | NP_001041631.1 | 265 |
| Secreted frizzled-related protein 1 | SFRP1 | NP_003003.3 | 266 |
| Secreted frizzled-related protein 4 | SFRP4 | NP_003005.2 | 267 |
| Sel-1 suppressor of lin-12-like (C. elegans) | SEL1L | NP_005056.3 | 268 |
| SH2 domain containing 1A | SH2D1A | NP_002342.1 | 269 |
| Signal transducer and activator of | STAT6 | NP_003144.3 | 270 |
| transcription 6, interleukin-4 induced | | | |
| Smoothened, frizzled family receptor | SMO | NP_005622.1 | 271 |
| SNW domain containing 1 | SNW1 | NP_036377.1 | 272 |
| Sonic hedgehog | SHH | NP_000184.1 | 273 |
| SRY (sex determining region Y)-box 17 | SOX17 | NP_071899.1 | 274 |
| Suppressor of fused homolog (Drosophila) | SUFU | NP_057253.2 | 275 |
| Transcription factor 7 (T-cell specific, | TCF7 | NP_003193.2 | 276 |
| HMG-box) | | | |
| Transcription factor 7-like 1 (T-cell | TCF7L1 | NP_112573.1 | 277 |
| specific, HMG-box) | | | |
| Transducin-like enhancer of split 1 (E(sp1) | TLE1 | NP_005068.2 | 278 |
| homolog, Drosophila) | | | |
| Vang-like 2 (van gogh, Drosophila) | VANGL2 | NP_065068.1 | 279 |
| V-erb-b2 erythroblastic leukemia viral | ERBB2 | NP_004439.2 | 280 |
| oncogene homolog 2, neuro/glioblastoma | | | |
| derived oncogene homolog (avian) | | | |
| V-myc myelocytomatosis viral oncogene | MYC | NP_002458.2 | 281 |
| homolog (avian) | | | |
| Wingless-type MMTV integration site | WNT2 | NP_003382.1 | 282 |
| family member 2 | | | |
| Wingless-type MMTV integration site | WNT1 | NP_005421.1 | 283 |
| family, member 1 | | | |
| Wingless-type MMTV integration site | WNT10A | NP_079492.2 | 284 |
| family, member 10A | | | |
| Wingless-type MMTV integration site | WNT11 | NP_004617.2 | 285 |
| family, member 11 | | | |
| Wingless-type MMTV integration site | WNT16 | NP_476509.1 | 286 |
| family, member 16 | | | |
| Wingless-type MMTV integration site | WNT2B | NP_078613.1 | 287 |
| family, member 2B | | | |
| Wingless-type MMTV integration site | WNT3 | NP_110380.1 | 288 |
| family, member 3 | | | |
| Wingless-type MMTV integration site | WNT3A | NP_149122.1 | 289 |
| family, member 3A | | | |
| Wingless-type MMTV integration site | WNT4 | NP_110388.2 | 290 |
| family, member 4 | | | |
| Wingless-type MMTV integration site | WNT5A | NP_003383.2 | 291 |
| family, member 5A | | | |
| Wingless-type MMTV integration site | WNT5B | NP_110402.2 | 292 |
| family, member 5B | | | |
| Wingless-type MMTV integration site | WNT6 | NP_006513.1 | 293 |
| family, member 6 | | | |
| Wingless-type MMTV integration site | WNT7A | NP_004616.2 | 294 |
| family, member 7A | | | |
| Wingless-type MMTV integration site | WNT7B | NP_478679.1 | 295 |
| family, member 7B | | | |
| Wingless-type MMTV integration site | WNT8A | NP_490645.1 | 296 |
| family, member 8A | | | |
| Wingless-type MMTV integration site | WNT9A | NP_003386.1 | 297 |
| family, member 9A | | | |
| WNT inhibitory factor 1 | WIF1 | NP_009122.2 | 298 |
| WNT1 inducible signaling pathway protein 1 | WISP1 | NP_003873.1 | 299 |
| Zic family member 2 | ZIC2 | NP_009060.2 | 300 |

### Natural Antagonists

A number of natural antagonists function to regulate development in vivo, such as the bone morphogenic protein (BMP) antagonists chordin, noggin, gremlin, sclerostin, and twisted gastrulation. The cysteine knot motif is present in many of these antagonists, and they mostly prevent the ligand from interacting with the receptor. Some antagonists prevent the processing of the mature ligand. And while one monoclonal antibody against sclerostin, also a powerful Wnt pathway inhibitor, is under clinical investigation as a new approach to increase bone mass in osteoporosis, there remains a need for antibodies directed to other natural antagonists.

For example, Dickkopf1 (DKK1) and secreted Frizzled-related protein (SFRP1) are two such inhibitors that negatively regulate bone mass and could be antagonized in osteoporosis. Another natural antagonists which may be targeted is follistatin. Follistatin is an activin-binding protein, interacting with high affinity and blocking activin-dependent signal transduction. The follistatin gene is also upregulated by activin signaling, providing negative feedback inhibition to the pathway. Follistatin expression is relatively ubiquitous throughout the body and the activin-follistatin system is implicated in multiple disorders in a variety of tissues making follistatin an attractive target for potential therapeutics (Aoki, F. et al., Therapeutic potential of follistatin to promote tissue regeneration and prevent tissue fibrosis. Endocr J. 2007 December; 54(6):849-54. Epub 2007 Oct. 15).

GDAs of the present invention may be designed to target such natural antagonists. These compositions would function to relieve signaling inhibition by blocking the inhibitor (natural antagonist) with the ultimate result being the release of the growth factor.

Natural antagonists which may be used to raise antibodies include those listed in Table 7.

TABLE 7
Natural Antagonist Targets

| | | Accession | SEQ |
|---|---|---|---|
| Name | Symbol | Number | ID NO |
| Dickkopf 1 homolog | DKK1 | NP_036374.1 | 176 |
| Secreted frizzled-related protein 1 | SFRP1 | NP_003003.3 | 266 |
| Chordin | CHRD | NP_003732.2 | 301 |
| Noggin | NOG | NP_005441.1 | 302 |
| Gremlin 1 | GREM1 | NP_037504.1 | 303 |
| Gremlin 2 | GREM2 | NP_071914.3 | 304 |
| Sclerostin | SOST | NP_079513.1 | 305 |
| Twisted gastrulation homolog 1 | TWSG1 | NP_065699.1 | 306 |
| Follistatin | FST | NP_037541.1 | 307 |
| Follistatin-like 1 | FSTL1 | NP_009016.1 | 308 |
| Follistatin-like 3 | FSTL3 | NP_005851.1 | 309 |
| Follistatin-like 4 | FSTL4 | NP_055897.1 | 310 |
| Follistatin-like 5 | FSTL5 | NP_064501.2 | 311 |
| Cerebrus 1 | CER1 | NP_005445.1 | 312 |
| Sclerostin domain containing 1 | SOSTDC1 | NP_056279.1 | 313 |
| DAN domain family, member 5 | DAND5 | NP_689867.1 | 314 |
| Neuroblastoma, suppression of | NBL1 | NP_877421.2 | 315 |
| tumorigenicity 1 | | | |
| BMP binding endothelial regulator | BMPER | NP_597725.1 | 316 |

### III. Methods and Uses

### Therapeutics

Compositions and methods of the invention may be used to treat a wide variety of disorders and conditions. These include, but are not limited to, fibrosis, anemia of the aging, cancer, facilitation of rapid hematopoiesis following chemotherapy, bone healing, endothelial proliferation syndromes and the orphan indications Marfan's syndrome, Camurati-Engelmann disease. Efficacy of treatment or amelioration of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. In connection with the administration of a GDA or pharmaceutical composition thereof, “effective against” for example a cancer, indicates that administration in a clinically appropriate manner results in a beneficial effect for at least a statistically significant fraction of patients, such as a improvement of symptoms, a cure, a reduction in disease load, reduction in tumor mass or cell numbers, extension of life, improvement in quality of life, or other effect generally recognized as positive by medical doctors familiar with treating the particular type of cancer.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for a given GDA drug or formulation of that drug can also be judged using an experimental animal model for the given disease as known in the art. When using an experimental animal model, efficacy of treatment is evidenced when a statistically significant change is observed.

### Therapeutics for Fibrosis

A major application of antagonists to TGF-beta is the treatment of fibrosis. TGF-beta is recognized as the central orchestrator of the fibrotic response.

One interesting aspect of fibrosis is that multiple members of the TGF-beta family can specifically antagonize each other's action. For instance, the BMP pathway can antagonize the effects of TGF-beta on fibrosis. BMPs are known to activate SMAD1, 5, and 8, in contrast to TGF-beta that activates SMAD2 and 3. These SMAD signaling transcription factors compete for the shared SMAD4 that dimerizes with each.

In models of experimental and renal fibrosis, TGF-beta is upregulated and BMP7 is downregulated. Even in a normal kidney, BMP7 has been shown to suppress fibronectin and collagen III. BMP7 appears to protect the kidney from fibrosis in several kidney damage models. Developing methods to induce BMP signal transduction may alter fibrosis.

Fibrosis is a common sequela of many types of tissue destructive diseases. When new space is created by the disruption of differentiated cells, progenitors or stem cells that normally occupy a niche in the tissue, the default pathway appears to be the proliferation of connective tissue cells, e.g. fibroblasts, to fill in the empty space. This is accompanied by the production of extracellular matrix constituents including collagens that result in scarring and permanent effacement of the tissue.

A difficult aspect of fibrosis is its chronicity, which may require continued therapy until the underlying destruction of parenchymal cells is terminated or the cells are replaced by stem cell pools, or by transplantation. Fibrosis is thought to be much easier to arrest than to reverse. The TGF-beta family is of central importance in regulating the growth of fibroblastic cells and the production of extracellular matrix constituents including collagen. Integrins αᵥβ₆ and αᵥβ₈ are required for activation of TGF-beta1 and 3. The integrin VLA-1 is a receptor for collagen and is expressed on lymphocytes only late after their activation and is strongly implicated in the development of fibrotic disease.

In some embodiments, GDA antibodies are designed to block integrin αᵥβ₆ activation of TGF-beta for inhibiting fibrosis. In some embodiments, GDA antibodies are designed to target interaction sites between GPCs and LTBP while leaving interaction sites between GPCs and GARP unaffected. Such GDA antibodies may act as inhibitory antibodies, preventing growth factor signaling and inhibiting fibrosis.

Assays useful in determining the efficacy of the GDAs for the treatment of fibrosis include histological assays for counting fibroblasts and basic immunohistochemical analyses known in the art.

Animal models are also available for analysis of the efficacy of GDAs in fibrosis treatment. On such model is the bleomycin induced lung injury model as described by Horan et al. (Horan G. S. et al., Partial inhibition of integrin alpha(v)beta6 prevents pulmonary fibrosis without exacerbating inflammation. Am J Respir Crit Care Med, 2008 Jan. 1; 177(1):56-65. Epub 2007 Oct. 4). In this model, SV129 mice are tracheally exposed to bleomycin which results in the development of lung fibrosis. With this model, potential therapeutics are administered through intraperitoneal injections while postmortem lung tissue or bronchoalveolar lavage collections can be assayed for levels of hydroxyproline as an indicator of fibrotic activity. Using the same technique, mice carrying a luciferase reporter gene, driven by the collagen Iα2 gene promoter may be used in the model so that fibrotic activity may be determined by luciferase activity assay as a function of collagen gene induction.

A well established model of renal fibrosis is the unilateral ureteral obstruction (UUO) model. In this model, mice are subjected to proximal ureteral ligation. After a period of hours to days, fibrosis is examined in the regions blocked by ligation. In one example, this method was utilized by Meng, X. M. et al. (Meng, X. M. et al., Smad2 Protects against TGF-beta/Smad3-Mediated Renal Fibrosis. J Am Soc Nephrol. 2010 September; 21(9):1477-87. Epub 2010 Jul. 1) to examine the role of Smad2 in renal fibrosis. Smad2 is an intracellular member of the TGF-beta cell signaling pathway.

### Therapeutics for Anemia, Thrombocytopenia and Neutropenia

In one embodiment, GDAs may be designed to treat patients suffering from anemia (the loss of red blood cells), thrombocytopenia (a decrease in the number of platelets) and/or neutropenia (a decrease in the number of neutrophils).

During chemotherapy, cell division is temporarily halted to prevent the growth and spread of cancerous cells. An unfortunate side effect is the loss of red blood cells, platelets and white blood cells which depend on active cell division of bone marrow cells. BMPs are important regulators of bone health and healing (Lissenberg-Thunnissen, S. N. et al. Use and efficacy of bone morphogenetic proteins in fracture healing. Int Orthop. 2011 September; 35(9):1271-80). GDAs of the present invention may function as BMP (preferably BMP2 and/or BMP7) agonists, thereby speeding up the recovery bone marrow cells and in turn, the production of neutrophil and platelet function after chemotherapy. Such GDAs may be important therapeutics for individuals undergoing chemotherapy or recovering from its effects.

A particularly interesting application for antagonizing BMP function is in the treatment of anemia of aging. Iron transport in the body is regulated by pathways studied for many years. Hepcidin is an acute phase protein that is aberrantly upregulated in chronic inflammation in the elderly and leads to iron imbalance. Hepcidin binds to the iron exporter ferroportin, and triggers the internalization of the exporter. Cells in many tissues are locked in a state of iron overload, and unable to deliver the iron to the erythron that requires it to make hemoglobin. Providing iron to these patients has no effect except to make the iron overload worse. Millions of patients have anemia of chronic disease, and no therapy is currently available. Hepcidin synthesis is signaled by BMP6 in conjunction with neogenin and hemojuvelin on liver cell surfaces (Zhang A. S. et al., Control of systemic iron homeostasis by the hemojuvelin-hepcidin axis. Adv Nutr. 2010 November; 1(1):38-45. Epub 2010 Nov. 16.). Hemojuvelin is a highly conserved protein that was only recently identified and to which few antibodies have been described. Its expression is limited to liver and skeletal muscle, in both of which it plays a role in iron regulation, with liver being the most important site.

Consequently, an antibody that would bind to hemojuvelin and block its interaction either with BMP6 or with neogenin would be a highly specific antagonist to prevent anemia of the aging. Such antibodies are contemplated herein.

Mouse as well as human models are known in the art to enable the optimization of such antibodies. Such models include diet-induced models of iron deficiency. In mice, restriction to an iron free diet and demineralized water for a period of 3 or more weeks is sufficient to induce iron deficiency (Kautz, L. et al., Iron regulates phosphorylation of Smad1/5/8 and gene expression of Bmp6, Smad7, Id1, and Atoh8 in the mouse liver. Blood. 2008 Aug. 15; 112(4):1503-9. Epub 2008 Jun. 6). Iron overload in mice can be induced through diet supplementation with carbonyl iron for a period of about 8 months. As an example, 3% carbonyl iron supplementation during this period results in about a 10-fold increase in serum iron concentration (Pigeon, C. et al., A new mouse liver-specific gene, encoding a protein homologous to human antimicrobial peptide hepcidin, is overexpressed during iron overload. J Biol Chem. 2001 Mar. 16; 276(11):7811-9. Epub 2000 Dec. 11).

Methods for studying elevated iron levels in humans are also available. One such study by Lin L, et al (Lin, L. et al., Iron transferrin regulates hepcidin synthesis in primary hepatocyte culture through hemojuvelin and BMP2/4. Blood. 2007 Sep. 15; 110(6):2182-9. Epub 2007 May 31) provided healthy volunteers with 65 mg of iron in the form of ferrous sulfate (Nature Made, Mission Hills, Calif.). Mouse strains with altered hepcidin expression are also known in the art and include the Bmp6-null (Bmp6^{m1Rob}) mouse (Andriopoulos, B. Jr. et al., BMP6 is a key endogenous regulator of hepcidin expression and iron metabolism. Nat Genet. 2009 April; 41(4):482-7. Epub 2009 Mar. 1) as well as the hemojuvelin-null (Hjv −/−) mouse (Huang, F. W. et al., A mouse model of juvenile hemochromatosis. J Clin Invest. 2005 August; 115(8):2187-91) that lack expression of BMP6 and hemojuvelin respectively. When compared with wildtype mice, young Bmp6-null mice exhibit a significant reduction (10-fold) in hepatic hepcidin mRNA levels and increased levels of serum iron. Hemojuvelin-null mice suffer from a rapid and systemic accumulation of iron. In addition, hepatic levels of hepcidin are downregulated.

For most BMPs, subsequent release of the growth factor would occur in the presence of receptors. Numerous proteins that antagonize BMPs could alternatively be targeted with inhibitory GDA antibodies, to activate BMP signaling.

Assays for the detection and or determination of efficacy of antibodies directed to BMP or hemojuvelin signaling include standard Smad signaling assays, measurement of iron metabolism, red cell indices, enzyme-linked immunosorbent assays (ELISA), gene and protein expression analyses, determining the shape of cells, hemoglobin content, and the like, each of which is known to those skilled in the art. Muscle wasting, satellite cell count and muscle mass determinations may also be used.

### Therapeutics for Cancer

Various cancers may be treated with the GDAs of the present invention. For example, a composition containing a GDA is used for treatment of a cancer. As used herein, cancer refers to any of various malignant neoplasms characterized by the proliferation of anaplastic cells that tend to invade surrounding tissue and metastasize to new body sites and also refers to the pathological condition characterized by such malignant neoplastic growths. A cancer can be a tumor or hematological malignancy, and includes but is not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus.

Leukemias, or cancers of the blood or bone marrow that are characterized by an abnormal proliferation of white blood cells i.e., leukocytes, can be divided into four major classifications including Acute lymphoblastic leukemia (ALL), Chronic lymphocytic leukemia (CLL), Acute myelogenous leukemia or acute myeloid leukemia (AML) (AML with translocations between chromosome 10 and 11 [t(10, 11)], chromosome 8 and 21 [t(8; 21)], chromosome 15 and 17 [t(15; 17)], and inversions in chromosome 16 [inv(16)]; AML with multilineage dysplasia, which includes patients who have had a prior myelodysplastic syndrome (MDS) or myeloproliferative disease that transforms into AML; AML and myelodysplastic syndrome (MDS), therapy-related, which category includes patients who have had prior chemotherapy and/or radiation and subsequently develop AML or MDS; d) AML not otherwise categorized, which includes subtypes of AML that do not fall into the above categories; and e) Acute leukemias of ambiguous lineage, which occur when the leukemic cells cannot be classified as either myeloid or lymphoid cells, or where both types of cells are present); and Chronic myelogenous leukemia (CML).

The types of carcinomas include, but are not limited to, papilloma/carcinoma, choriocarcinoma, endodermal sinus tumor, teratoma, adenoma/adenocarcinoma, melanoma, fibroma, lipoma, leiomyoma, rhabdomyoma, mesothelioma, angioma, osteoma, chondroma, glioma, lymphoma/leukemia, squamous cell carcinoma, small cell carcinoma, large cell undifferentiated carcinomas, basal cell carcinoma and sinonasal undifferentiated carcinoma.

The types of sarcomas include, but are not limited to, soft tissue sarcoma such as alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, synovial sarcoma, and Askin's tumor, Ewing's sarcoma (primitive neuroectodermal tumor), malignant hemangioendothelioma, malignant schwannoma, osteosarcoma, and chondrosarcoma.

The invention further relates to the use of a GDA or a pharmaceutical composition thereof, e.g., for treating a cancer, in combination with other pharmaceuticals and/or other therapeutic methods, e.g., with known pharmaceuticals and/or known therapeutic methods, such as, for example, those which are currently employed for treating these disorders. For example, the GDA or pharmaceutical composition thereof can also be administered in conjunction with one or more additional anti-cancer treatments, such as biological, chemotherapy and radiotherapy. Accordingly, a treatment can include, for example, imatinib (Gleevac), all-trans-retinoic acid, a monoclonal antibody treatment (gemtuzumab, ozogamicin), chemotherapy (for example, chlorambucil, prednisone, prednisolone, vincristine, cytarabine, clofarabine, farnesyl transferase inhibitors, decitabine, inhibitors of MDR1), rituximab, interferon-α, anthracycline drugs (such as daunorubicin or idarubicin), L-asparaginase, doxorubicin, cyclophosphamide, doxorubicin, bleomycin, fludarabine, etoposide, pentostatin, or cladribine), bone marrow transplant, stem cell transplant, radiation therapy, anti-metabolite drugs (methotrexate and 6-mercaptopurine), or any combination thereof.

Radiation therapy (also called radiotherapy, X-ray therapy, or irradiation) is the use of ionizing radiation to kill cancer cells and shrink tumors. Radiation therapy can be administered externally via external beam radiotherapy (EBRT) or internally via brachytherapy. The effects of radiation therapy are localized and confined to the region being treated. Radiation therapy may be used to treat almost every type of solid tumor, including cancers of the brain, breast, cervix, larynx, lung, pancreas, prostate, skin, stomach, uterus, or soft tissue sarcomas. Radiation is also used to treat leukemia and lymphoma.

Chemotherapy is the treatment of cancer with drugs that can destroy cancer cells. In current usage, the term “chemotherapy” usually refers to cytotoxic drugs which affect rapidly dividing cells in general, in contrast with targeted therapy. Chemotherapy drugs interfere with cell division in various possible ways, e.g. with the duplication of DNA or the separation of newly formed chromosomes. Most forms of chemotherapy target all rapidly dividing cells and are not specific to cancer cells, although some degree of specificity may come from the inability of many cancer cells to repair DNA damage, while normal cells generally can.

Most chemotherapy regimens are given in combination. Exemplary chemotherapeutic agents include, but are not limited to, 5-FU Enhancer, 9-AC, AG2037, AG3340, Aggrecanase Inhibitor, Aminoglutethimide, Amsacrine (m-AMSA), Asparaginase, Azacitidine, Batimastat (BB94), BAY 12-9566, BCH-4556, Bis-Naphtalimide, Busulfan, Capecitabine, Carboplatin, Carmustaine+Polifepr Osan, cdk4/cdk2 inhibitors, Chlorombucil, CI-994, Cisplatin, Cladribine, CS-682, Cytarabine HCl, D2163, Dactinomycin, Daunorubicin HCl, DepoCyt, Dexifosamide, Docetaxel, Dolastain, Doxifluridine, Doxorubicin, DX8951f, E 7070, EGFR, Epirubicin, Erythropoietin, Estramustine phosphate sodium, Etoposide (VP16-213), Farnesyl Transferase Inhibitor, FK 317, Flavopiridol, Floxuridine, Fludarabine, Fluorouracil (5-FU), Flutamide, Fragyline, Gemcitabine, Hexamethylmelamine (HMM), Hydroxyurea (hydroxycarbamide), Ifosfamide, Interferon Alfa-2a, Interferon Alfa-2b, Interleukin-2, Irinotecan, ISI 641, Krestin, Lemonal DP 2202, Leuprolide acetate (LHRH-releasing factor analogue), Levamisole, LiGLA (lithium-gamma linolenate), Lodine Seeds, Lometexol, Lomustine (CCNU), Marimistat, Mechlorethamine HCl (nitrogen mustard), Megestrol acetate, Meglamine GLA, Mercaptopurine, Mesna, Mitoguazone (methyl-GAG; methyl glyoxal bis-guanylhydrazone; MGBG), Mitotane (o.p′-DDD), Mitoxantrone, Mitoxantrone HCl, MMI 270, MMP, MTA/LY 231514, Octreotide, ODN 698, OK-432, Oral Platinum, Oral Taxoid, Paclitaxel (TAXOL®), PARP Inhibitors, PD 183805, Pentostatin (2′ deoxycoformycin), PKC 412, Plicamycin, Procarbazine HCl, PSC 833, Ralitrexed, RAS Farnesyl Transferase Inhibitor, RAS Oncogene Inhibitor, Semustine (methyl-CCNU), Streptozocin, Suramin, Tamoxifen citrate, Taxane Analog, Temozolomide, Teniposide (VM-26), Thioguanine, Thiotepa, Topotecan, Tyrosine Kinase, UFT (Tegafur/Uracil), Valrubicin, Vinblastine sulfate, Vindesine sulfate, VX-710, VX-853, YM 116, ZD 0101, ZD 0473/Anormed, ZD 1839, ZD 9331.

Biological therapies use the body's immune system, either directly or indirectly, to fight cancer or to lessen the side effects that may be caused by some cancer treatments. In one sense, GDAs can be considered in this group of therapies in that it can stimulate immune system action against a tumor, for example. However, this approach can also be considered with other such biological approaches, e.g., immune response modifying therapies such as the administration of interferons, interleukins, colony-stimulating factors, other monoclonal antibodies, vaccines, gene therapy, and nonspecific immunomodulating agents are also envisioned as anti-cancer therapies to be combined with the GDAs.

Small molecule targeted therapy drugs are generally inhibitors of enzymatic domains on mutated, overexpressed, or otherwise critical proteins within the cancer cell, such as tyrosine kinase inhibitors imatinib (Gleevec/Glivec) and gefitinib (Iressa). Examples of monoclonal antibody therapies that can be used with a GDA or pharmaceutical composition thereof include, but are not limited to, the anti-HER2/neu antibody trastuzumab (Herceptin) used in breast cancer, and the anti-CD20 antibody rituximab, used in a variety of B-cell malignancies. The growth of some cancers can be inhibited by providing or blocking certain hormones. Common examples of hormone-sensitive tumors include certain types of breast and prostate cancers. Removing or blocking estrogen or testosterone is often an important additional treatment. In certain cancers, administration of hormone agonists, such as progestogens may be therapeutically beneficial.

Cancer immunotherapy refers to a diverse set of therapeutic strategies designed to induce the patient's own immune system to fight the tumor, and include, but are not limited to, intravesical BCG immunotherapy for superficial bladder cancer, vaccines to generate specific immune responses, such as for malignant melanoma and renal cell carcinoma, and the use of Sipuleucel-T for prostate cancer, in which dendritic cells from the patient are loaded with prostatic acid phosphatase peptides to induce a specific immune response against prostate-derived cells.

In some embodiments, GDA antibodies are designed to prevent T cell inhibition. Such antibodies may prevent the dissociation of growth factors from the prodomain of the GPC or from ECCM components including, but not limited to GARP.

Therapeutics for Bone Healing

The GDA compositions of the present invention may be used to treat bone disorders and/or improve bone healing or repair. Cellular remodeling of bone is a lifelong process that helps to maintain skeletal integrity. This process involves cycles of osteoclastic bone resorption and new bone formation that function to repair defects and areas of weakness in bone. TGF-beta family members, preferably BMPs, are thought to be important factors in coupling the processes of resorption and formation by osteoclasts. TGF-beta family members are prevalent in the bone matrix and upregulated by bone injury. TGF-beta family members are also believed to impart strength to the fully formed bone matrix, imparting resistance to fracture. The role of TGF-beta family members in bone remodeling makes them attractive targets for potential therapeutics to treat bone disorder and disease.

Numerous diseases and disorders affect bones and joints. Such diseases and disorders may be congenital, genetic and/or acquired. Such diseases and disorders include, but are not limited to, bone cysts, infectious arthritis, Paget's disease of the bone, Osgood-Schlatter disease, Kohler's bone disease, bone spurs (osteophytes), bone tumors, craniosynostosis, fibrodysplasia ossificans progressive, fibrous dysplasia, giant cell tumor of bone, hypophosphatasia, Klippel-Feil syndrome, metabolic bone disease, osteoarthritis, osteitis deformans, osteitis fibrosa cystica, osteitis pubis, condensing osteitis, osteitis condensans ilii, osteochondritis dissecans, osteochondroma, osteogenesis imperfecta, osteomalacia, osteomyelitis, osteopenia, osteopetrosis, osteoporosis, osteosarcoma, porotic hyperostosis, primary hyperparathyroidism, renal osteodystrophy and water on the knee.

Mouse models for evaluating the effectiveness of therapeutics on bone development and repair are well known in the art. In one such model demonstrated by Mohammad, et al. (Mohammad, K. S. et al., Pharmacologic inhibition of the TGF-beta type I receptor kinase has anabolic and anti-catabolic effects on bone. PLoS One. 2009; 4(4):e5275. Epub 2008 Apr. 16), inhibition of the TGF-beta type I receptor was carried out in C57Bl/6 mice through twice daily administration of a potent inhibitor, SD-208, by gavage. Subsequently, bone mineral density (BMD) was analyzed using a PIXImus mouse densitometer (GE Lunar II, Faxitron Corp., Wheeling, Ill.). Changes in BMD are expressed as a percentage change in the area scanned. The study found that after 6 weeks of treatment, male mice exhibited a 4.12% increase in bone accrual while female mice exhibited a 5.2% increase.

GDAs of the present invention may be directed toward therapies for simple and complex bone fractures and/or bone repair. In such treatments, GDAs may be introduced to the site of injury directly or through the incorporation into implantation devices and coated biomatrices. Additionally, treatments are contemplated in which a GDA is supplied together with its GPC in a treatment area and facilitates the slow release of growth factors from the GPCs.

### Therapeutics for Angiogenic and Endothelial Proliferation Conditions

The GDA compositions of the present invention may be used to treat angiogenic and endothelial proliferation syndromes, diseases or disorders. The term “angiogenesis”, as used herein refers to the formation and/or reorganization of new blood vessels. Angiogenic disease involves the loss of control over angiogenesis in the body. In such cases, blood vessel growth, formation or reorganization may be overactive (including during tumor growth and cancer where uncontrolled cell growth requires increases blood supply) or insufficient to sustain healthy tissues. Such conditions may include, but are not limited to angiomas, angiosarcomas, telangiectasia, lymphangioma, congenital vascular anomalies, tumor angiogenesis and vascular structures after surgery. Excessive angiogenesis is noted in cancer, macular degeneration, diabetic blindness, rheumatoid arthritis, psoriasis as well as many other conditions. Excessive angiogenesis is often promoted by excessive angiogenic growth factor expression. GDAs of the present invention may act to block growth factors involved in excessive angiogenesis. Alternatively, GDAs of the present invention may be utilized to promote growth factor signaling to enhance angiogenesis in conditions where angiogenesis is inhibited. Such conditions include, but are not limited to coronary artery disease, stroke, diabetes and chronic wounds.

### Therapeutics for Orphan Indications and Diseases

The GDA compositions of the present invention may be used to treat orphan indications and/or diseases. Such diseases include Marfan's syndrome. This syndrome is a connective tissue disorder, effecting bodily growth and development. Tissues and organs that are most severely compromised include the heart, blood vessels, bones, eyes, lungs and connective tissue surrounding the spinal chord. Unfortunately, the effects can be life threatening. Marfan's syndrome is caused by a genetic mutation in the gene that produces fibrillin, a major component of bodily connective tissue. Latent TGF-beta binding protein (LTBP) is an important regulator of TGF-beta signaling that exhibits close identity to fibrillin protein family members. Functional LTBP is required for controlling the release of active TGF-beta (Oklu, R. et al., The latent transforming growth factor beta binding protein (LTBP) family. Biochem J. 2000 Dec. 15; 352 Pt 3:601-10). In this embodiment, GDA compositions are designed to alter the release profile of TGF-beta. In such cases, the GDA antibody would be an inhibitory antibody.

Another indication is Camurati-Engelmann disease (CED). This disease primarily affects the bones, resulting in increased bone density. Especially affected are the long bones of the legs and arms; however, the bones of the skill and hips can also be affected. The disease results in leg and arm pain as well as a variety of other symptoms. CED is very rare, reported in approximately 200 individuals worldwide and is caused by a mutation in the TGF-beta gene. TGF-beta produced in the bodies of these individuals has a defective prodomain, leading to overactive TGF-beta signaling (Janssens, K. et al., Transforming growth factor-beta 1 mutations in Camurati-Engelmann disease lead to increased signaling by altering either activation or secretion of the mutant protein. J Biol Chem. 2003 Feb. 28; 278(9):7718-24. Epub 2002 Dec. 18). As described by Shi et al., (Shi, M. et al., Latent TGF-beta structure and activation. Nature. 2011 Jun. 15; 474(7351):343-9. doi: 10.1038/nature10152), among CED mutations, Y52H disrupts an α2-helix residue that cradles the TGF-beta fingers. The charge-reversal E140K and H193D mutations disrupt a pH-regulated salt bridge between Glu 140 and His 193 in the dimerization interface of the prodomain. Residue Arg 189 is substantially buried: it forms a cation-π bond with Tyr 142 and salt bridges across the dimer interface with residue Asp 197 of the ‘bowtie’ region of the growth-factor prodomain complex (GPC). Moreover, CED mutations in Cys 194 and Cys 196 demonstrate the importance of disulphide bonds in the bowtie region for holding TGF-beta in inactive form. In this embodiment, an inhibitory GDA antibody would serve to alleviate symptoms. Also in this embodiment, administration would be to the neonate subject.

In yet another embodiment, GDA antibodies are designed to treat hereditary hemorrhagic telangiectasia (HHT), a genetic blood vessel disorder affecting about 1 in 5,000 people. The mutated genes in HHT are modulators of TGF-beta signaling in the vascular endothelium. Affected individuals develop abnormal vascular structures ranging from dilated microvessels to enlarged arteriovenous malformations. The fragile walls of these vessels leave them susceptible to hemorrhage (Govani, F. S. et al., Hereditary haemorrhagic telangiectasia: a clinical and scientific review. Eur J Hum Genet. 2009 July; 17(7):860-71. Epub 2009 Apr. 1). In one form of the disorder, HHT is caused by a mutation in activin receptor-like kinase 1 (ALK1), an endothelial-specific TGF-beta type 1 receptor. The physiological ligand for this receptor is a TGF-beta family member, BMP9. Overexpression of BMP9 has been shown to reduce endothelial cell migration. In one embodiment, symptoms of HHT would be alleviated by altering BMP9 signaling.

### Therapeutics for Immune and Autoimmune Diseases and Disorders

The GDA compositions of the present invention may be used to treat immune and autoimmune disorders. Such disorders include, but are not limited to Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune pancreatitis, Autoimmune retinopathy, Autoimmune thrombocytopenic purpura (ATP), Autoimmune thyroid disease, Autoimmune urticaria, Axonal & neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Diabetes Type I, Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Glomerulonephritis, Goodpasture's syndrome, Granulomatosis with Polyangiitis (GPA) see Wegener's, Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Insulin-dependent diabetes (type1), Interstitial cystitis, Juvenile arthritis, Juvenile diabetes, Kawasaki syndrome, Lambert-Eaton syndrome, Large vessel vasculopathy, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple endocrine neoplasia syndromes, Multiple sclerosis, Myositis, Myasthenia gravis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polyendocrinopathies, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic Pulmonary fibrosis, Pyoderma gangrenosum, Pure red cell aplasia, Raynauds phenomenon, Reactive arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Small vessel vasculopathy, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Tubular autoimmune disorder, Ulcerative colitis, Undifferentiated connective tissue disease (UCTD), Uveitis, Vesiculobullous dermatosis, Vasculitis, Vitiligo and Wegener's granulomatosis (also known as Granulomatosis with Polyangiitis (GPA)).

TGF-beta plays an active role in leukocyte differentiation, proliferation and activation making it an important factor in immune and autoimmune diseases. Additionally, TGF-beta promotes chemotaxis of leukocytes and influences adhesion molecule-mediated localization. A role for TGF-beta in cardiac, pulmonary and gastric inflammation has been demonstrated. Furthermore, Smad3-deficient mice are prone to chronic mucosal infections as a result of T-cell activation impairment and reduced mucosal immunity (Blobe, G. C. et al., Role of transforming growth factor beta in human disease. N Engl J Med. 2000 May 4; 342(18):1350-8). As an immunosuppressant, TGF-beta has been shown to both inhibit the function of inflammatory cells as well as enhance the function of regulatory T cells. Recent studies have shown that the latent TGF-beta growth factor prodomain complex (GPC) binds to regulatory T cells through an interaction with the Glycoprotein-A repetitions anonymous protein (GARP). This interaction provides the platform necessary to release active TGF-beta from the GPC in an integrin-dependent manner (Wang, R. et al., GARP regulates the bioavailability and activation of TGFβ. Mol Biol Cell. 2012 March; 23(6):1129-39. Epub 2012 Jan. 25). In one embodiment, a GDA may be used for the treatment of an immune or autoimmune disorder. In another embodiment, a GDA may specifically target GARP-bound GPC, GARP or the interaction site between GARP and the GPC. In one embodiment, GDA antibodies are designed to promote release of growth factors (including, but not limited to TGF-beta) from GARP-bound GPCs while not affecting growth factor release from LTBP-bound GPCs. Treatment of immune and autoimmune disorders with the GDA compositions may be in combination with standard of care (SOC) or synergistic combinations or with companion diagnostics.

### Therapeutics for Infectious Agents

The invention further relates to the use of a GDA for treatment of an infectious disease or disorder, for example, in a subject having an infection. In some preferred embodiments the subject has an infection or is at risk of having an infection. An “infection” as used herein refers to a disease or condition attributable to the presence in a host of a foreign organism or agent that reproduces within the host. Infections typically involve breach of a normal mucosal or other tissue barrier by an infectious organism or agent. A subject that has an infection is a subject having objectively measurable infectious organisms or agents present in the subject's body. A subject at risk of having an infection is a subject that is predisposed to develop an infection. Such a subject can include, for example, a subject with a known or suspected exposure to an infectious organism or agent. A subject at risk of having an infection also can include a subject with a condition associated with impaired ability to mount an immune response to an infectious organism or agent, e.g., a subject with a congenital or acquired immunodeficiency, a subject undergoing radiation therapy or chemotherapy, a subject with a burn injury, a subject with a traumatic injury, a subject undergoing surgery or other invasive medical or dental procedure.

Infections are broadly classified as bacterial, viral, fungal, or parasitic based on the category of infectious organism or agent involved. Other less common types of infection are also known in the art, including, e.g., infections involving rickettsiae, mycoplasmas, and agents causing scrapie, bovine spongiform encephalopathy (BSE), and prion diseases (e.g., kuru and Creutzfeldt-Jacob disease). Examples of bacteria, viruses, fungi, and parasites which cause infection are well known in the art. An infection can be acute, subacute, chronic, or latent, and it can be localized or systemic. As defined herein, a “chronic infection” refers to those infections that are not cleared by the normal actions of the innate or adaptive immune responses and persist in the subject for a long duration of time, on the order of weeks, months, and years. A chronic infection may reflect latency of the infectious agent, and may be include periods in which no infectious symptoms are present, i.e., asymptomatic periods. Examples of chronic infections include, but are not limited to, HIV infection and herpesvirus infections. Furthermore, an infection can be predominantly intracellular or extracellular during at least one phase of the infectious organism's or agent's life cycle in the host.

Exemplary viruses include, but are not limited to: Retroviridae (e.g., human immunodeficiency viruses, such as HIV-1 (also referred to as HTLV-III), HIV-2, LAV or HTLV-III/LAV, or HIV-III, and other isolates, such as HIV-LP; Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); adenovirus; Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae (e.g., reoviruses, orbiviurses and rotaviruses, i.e., Rotavirus A, Rotavirus B. Rotavirus C); Birnaviridae; Hepadnaviridae (Hepatitis A and B viruses); Parvoviridae (parvoviruses); Papovaviridae (papilloma viruses, polyoma viruses); Adenoviridae (most adenoviruses); Herpesviridae (herpes simplex virus (HSV) 1 and 2, Human herpes virus 6, Human herpes virus 7, Human herpes virus 8, varicella zoster virus, cytomegalovirus (CMV), herpes virus; Epstein-Barr virus; Rous sarcoma virus; West Nile virus; Japanese equine encephalitis, Norwalk, papilloma virus, parvovirus B19; Poxyiridae (variola viruses, vaccinia viruses, pox viruses); and Iridoviridae (e.g., African swine fever virus); Hepatitis D virus, Hepatitis E virus, and unclassified viruses (e.g., the etiological agents of Spongiform encephalopathies, the agent of delta hepatitis (thought to be a defective satellite of hepatitis B virus), the agents of non-A, non-B hepatitis (class 1=enterally transmitted; class 2=parenterally transmitted (i.e., Hepatitis C); Norwalk and related viruses, and astroviruses).

Bacteria include both Gram negative and Gram positive bacteria. Examples of Gram positive bacteria include, but are not limited to Pasteurella species, Staphylococci species, and Streptococcus species. Examples of Gram negative bacteria include, but are not limited to, Escherichia coli, Pseudomonas species, and Salmonella species. Specific examples of infectious bacteria include but are not limited to: Helicobacter pyloris, Borrelia burgdorferi, Legionella pneumophilia, Mycobacteria spp. (e.g., M. tuberculosis, M. avium, M. intracellulare, M. kansasii, M. gordonae, M. leprae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic spp.), Streptococcus pneumoniae, pathogenic Campylobacter spp., Enterococcus spp., Haemophilus influenzae (Hemophilus influenza B, and Hemophilus influenza non-typable), Bacillus anthracis, Corynebacterium diphtheriae, Corynebacterium spp., Erysipelothrix rhusiopathiae, Clostridium perfringens, Clostridium tetani, Enterobacter aerogenes, Klebsiella pneumoniae, Pasteurella multocida, Bacteroides spp., Fusobacterium nucleatum, Streptobacillus moniliformis, Treponema pallidum, Treponema pertenue, Leptospira, Rickettsia, Actinomyces israelii, meningococcus, pertussis, pneumococcus, Shigella, tetanus, Vibrio cholerae, yersinia, Pseudomonas species, Clostridia species, Salmonella typhi, Shigella dysenteriae, Yersinia pestis, Brucella species, Legionella pneumophila, Rickettsiae, Chlamydia, Clostridium perfringens, Clostridium botulinum, Staphylococcus aureus, Pseudomonas aeruginosa, Cryptosporidium parvum, Streptococcus pneumoniae, and Bordetella pertussis.

Exemplary fungi and yeast include, but are not limited to, Cryptococcus neoformans, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Aspergillus fumigatus, Aspergillus flavus, Blastomyces dermatitidis, Aspergillus clavatus, Cryptococcus neoformans, Chlamydia trachomatis, Coccidioides immitis, Cryptococcus laurentii, Cryptococcus albidus, Cryptococcus gattii, Nocardia spp, Histoplasma capsulatum, Pneumocystis jirovecii (or Pneumocystis carinii), Stachybotrys chartarum, and any combination thereof.

Exemplary parasites include, but are not limited to: Entamoeba histolytica; Plasmodium species (Plasmodium falciparum, Plasmodium malariae, Plasmodium ovale, Plasmodium vivax), Leishmania species (Leishmania tropica, Leishmania braziliensis, Leishmania donovani), Toxoplasmosis (Toxoplasma gondii), Trypanosoma gambiense, Trypanosoma rhodesiense (African sleeping sickness), Trypanosoma cruzi (Chagas' disease), Helminths (flat worms, round worms), Babesia microti, Babesia divergens, Giardia lamblia, and any combination thereof.

The invention further relates to the use of a GDA for the treatment of an infectious disease, such as hepatitis B or a chronic bacterial infection, in combination with other pharmaceuticals and/or other therapeutic methods, e.g., with known pharmaceuticals and/or known therapeutic methods, such as, for example, those which are currently employed for treating such infectious diseases or disorders (e.g., antibiotics, anti-viral agents). For example, in certain embodiments, a GDA is administered in combination with an antibacterial agent. Examples of anti-bacterial agents useful for the methods described herein include, but are not limited to, natural penicillins, semi-synthetic penicillins, clavulanic acid, cephalolsporins, bacitracin, ampicillin, carbenicillin, oxacillin, azlocillin, mezlocillin, piperacillin, methicillin, dicloxacillin, nafcillin, cephalothin, cephapirin, cephalexin, cefamandole, cefaclor, cefazolin, cefuroxine, cefoxitin, cefotaxime, cefsulodin, cefetamet, cefixime, ceftriaxone, cefoperazone, ceftazidine, moxalactam, carbapenems, imipenems, monobactems, eurtreonam, vancomycin, polymyxin, amphotericin B, nystatin, imidazoles, clotrimazole, miconazole, ketoconazole, itraconazole, fluconazole, rifampins, ethambutol, tetracyclines, chloramphenicol, macrolides, aminoglycosides, streptomycin, kanamycin, tobramycin, amikacin, gentamicin, tetracycline, minocycline, doxycycline, chlortetracycline, erythromycin, roxithromycin, clarithromycin, oleandomycin, azithromycin, chloramphenicol, quinolones, co-trimoxazole, norfloxacin, ciprofloxacin, enoxacin, nalidixic acid, temafloxacin, sulfonamides, gantrisin, and trimethoprim; Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefinenoxime Hydrochloride; Cefinetazole; Cefinetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Inipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; and Zorbamycin.

In other embodiments, administration of a GDA is performed in combination with an anti-viral medicament or agent. Exemplary antiviral agents useful for the methods described herein include, but are not limited to, immunoglobulins, amantadine, interferon, nucleoside analogues, and protease inhibitors. Specific examples of antiviral agents include but are not limited to Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscamet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; and Zinviroxime.

In other embodiments, administration of a GDA is performed in combination with an anti-fungal medicament or agent. An “antifungal medicament” is an agent that kills or inhibits the growth or function of infective fungi. Anti-fungal medicaments are sometimes classified by their mechanism of action. Some anti-fungal agents function as cell wall inhibitors by inhibiting glucose synthase, other antifungal agents function by destabilizing membrane integrity, and other antifungal agents function by breaking down chitin (e.g., chitinase) or immunosuppression (501 cream). Thus, exemplary antifungal medicaments useful for the methods described herein include, but are not limited to, imidazoles, 501 cream, and Acrisorcin, Ambruticin, Amorolfine, Amphotericin B, Azaconazole, Azaserine, Basifungin, BAY 38-9502, Bifonazole, Biphenamine Hydrochloride, Bispyrithione Magsulfex, Butenafine, Butoconazole Nitrate, Calcium Undecylenate, Candicidin, Carbol-Fuchsin, Chitinase, Chlordantoin, Ciclopirox, Ciclopirox Olamine, Cilofungin, Cisconazole, Clotrimazole, Cuprimyxin, Denofungin, Dipyrithione, Doconazole, Econazole, Econazole Nitrate, Enilconazole, Ethonam Nitrate, Fenticonazole Nitrate, Filipin, FK 463, Fluconazole, Flucytosine, Fungimycin, Griseofulvin, Hamycin, Isoconazole, Itraconazole, Kalafungin, Ketoconazole, Lomofungin, Lydimycin, Mepartricin, Miconazole, Miconazole Nitrate, MK 991, Monensin, Monensin Sodium, Naftifine Hydrochloride, Neomycin Undecylenate, Nifuratel, Nifurmerone, Nitralamine Hydrochloride, Nystatin, Octanoic Acid, Orconazole Nitrate, Oxiconazole Nitrate, Oxifungin Hydrochloride, Parconazole Hydrochloride, Partricin, Potassium Iodide, Pradimicin, Proclonol, Pyrithione Zinc, Pyrrolnitrin, Rutamycin, Sanguinarium Chloride, Saperconazole, Scopafungin, Selenium Sulfide, Sertaconazole, Sinefungin, Sulconazole Nitrate, Terbinafine, Terconazole, Thiram, Ticlatone, Tioconazole, Tolciclate, Tolindate, Tolnaftate, Triacetin, Triafungin, UK 292, Undecylenic Acid, Viridofulvin, Voriconazole, Zinc Undecylenate, and Zinoconazole Hydrochloride.

In further embodiments, administration of a GDA is performed with an anti-parasitic medicament or agent. An “antiparasitic medicament” refers to an agent that kills or inhibits the growth or function of infective parasites. Examples of antiparasitic medicaments, also referred to as parasiticides, useful for the methods described herein include, but are not limited to, albendazole, amphotericin B, benznidazole, bithionol, chloroquine HCl, chloroquine phosphate, clindamycin, dehydroemetine, diethylcarbamazine, diloxanide furoate, doxycycline, eflomithine, furazolidaone, glucocorticoids, halofantrine, iodoquinol, ivermectin, mebendazole, mefloquine, meglumine antimoniate, melarsoprol, metrifonate, metronidazole, niclosamide, nifurtimox, oxamniquine, paromomycin, pentamidine isethionate, piperazine, praziquantel, primaquine phosphate, proguanil, pyrantel pamoate, pyrimethanmine-sulfonamides, pyrimethanmine-sulfadoxine, quinacrine HCl, quinine sulfate, quinidine gluconate, spiramycin, stibogluconate sodium (sodium antimony gluconate), suramin, tetracycline, thiabendazole, timidazole, trimethroprim-sulfamethoxazole, and tryparsamide, some of which are used alone or in combination with others.

A GDA and an additional therapeutic agent may be administered in combination in the same composition, e.g., parenterally, or the additional therapeutic agent may be administered as part of a separate composition or by another method described herein.

### Veterinary Applications

It is contemplated that the compositions and methods of the invention will find utility in the area of veterinary care including the care and treatment of non-human vertebrates. As described herein, the term “non-human vertebrate” includes all vertebrates with the exception of Homo sapiens, including wild and domesticated species such as companion animals and livestock. Non-human vertebrates include mammals, such as alpaca, banteng, bison, camel, cat, cattle, deer, dog, donkey, gayal, goat, guinea pig, horse, llama, mule, pig, rabbit, reindeer, sheep water buffalo, and yak. Livestock includes domesticated animals raised in an agricultural setting to produce materials such as food, labor, and derived products such as fiber and chemicals. Generally, livestock includes all mammals, avians and fish having potential agricultural significance. In particular, four-legged slaughter animals include steers, heifers, cows, calves, bulls, cattle, swine and sheep.

### Bioprocessing

In one embodiment of the invention are methods for producing a biological product in a host cell by contacting the cell with a GDA (such as an antibody or fusion protein) capable of modulating expression of a target gene, or altering the levels of growth factor signaling molecules wherein such modulation or alteration enhances production of the biological product. According to the present invention, bioprocessing methods may be improved by using one or more of the GDAs of the present invention. They may also be improved by supplementing, replacing or adding one or more GDAs.

### IV. Pharmaceutical Compositions

The pharmaceutical compositions described herein can be characterized by one or more of bioavailability, therapeutic window and/or volume of distribution.

### Bioavailability

In one embodiment, the pharmaceutical composition consists of a GDA complex with a GPC. In such an embodiment, the GDA:GPC complex may be implanted at a desired therapeutic site where steady dissociation of the GPC and the growth factor from the GDA may occur over a desired period of time. In another embodiment, implantation of a GDA:GPC complex may be in association with a sponge or bone-like matrix. Such implantation sites may include, but are not limited to dental implant sites and sites of bone repair.

In another embodiment, the GPC is made in furin-deficient cells. Such underprocessed GPCs may be useful for treatment in areas where release is slowed due to the fact that furin cleavage in vivo is rate-limiting during GPC processing. In a further embodiment, one or both of the tolloid or furin sites in the GPC are mutated, to slow action of endogenous tolloid and/or furin proteases, resulting in even slower release at the site of implantation.

GDAs, when formulated into a composition with a delivery/formulation agent or vehicle as described herein, can exhibit an increase in bioavailability as compared to a composition lacking a delivery agent as described herein. As used herein, the term “bioavailability” refers to the systemic availability of a given amount of GDAs administered to a mammal. Bioavailability can be assessed by measuring the area under the curve (AUC) or the maximum serum or plasma concentration (Cₘₐₓ) of the unchanged form of a compound following administration of the compound to a mammal. AUC is a determination of the area under the curve plotting the serum or plasma concentration of a compound along the ordinate (Y-axis) against time along the abscissa (X-axis). Generally, the AUC for a particular compound can be calculated using methods known to those of ordinary skill in the art and as described in G. S. Banker, Modern Pharmaceutics, Drugs and the Pharmaceutical Sciences, v. 72, Marcel Dekker, New York, Inc., 1996, herein incorporated by reference.

The Cₘₐₓ value is the maximum concentration of the compound achieved in the serum or plasma of a mammal following administration of the compound to the mammal. The Cₘₐₓ value of a particular compound can be measured using methods known to those of ordinary skill in the art. The phrases “increasing bioavailability” or “improving the pharmacokinetics,” as used herein mean that the systemic availability of a GDA, measured as AUC, Cₘₐₓ, or Cₘᵢₙ in a mammal is greater, when co-administered with a delivery agent as described herein, than when such co-administration does not take place. In some embodiments, the bioavailability of the GDA can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

### Therapeutic Window

GDAs, when formulated into a composition with a delivery agent as described herein, can exhibit an increase in the therapeutic window of the administered GDA composition as compared to the therapeutic window of the administered GDA composition lacking a delivery agent as described herein. As used herein “therapeutic window” refers to the range of plasma concentrations, or the range of levels of therapeutically active substance at the site of action, with a high probability of eliciting a therapeutic effect. In some embodiments, the therapeutic window of the GDA when co-administered with a delivery agent as described herein can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

### Volume of Distribution

GDAs, when formulated into a composition with a delivery agent as described herein, can exhibit an improved volume of distribution (V_{dist}), e.g., reduced or targeted, relative to a composition lacking a delivery agent as described herein. The volume of distribution (V_{dist}) relates the amount of the drug in the body to the concentration of the drug in the blood or plasma. As used herein, the term “volume of distribution” refers to the fluid volume that would be required to contain the total amount of the drug in the body at the same concentration as in the blood or plasma: V_{dist} equals the amount of drug in the body/concentration of drug in blood or plasma. For example, for a 10 mg dose and a plasma concentration of 10 mg/L, the volume of distribution would be 1 liter. The volume of distribution reflects the extent to which the drug is present in the extravascular tissue. A large volume of distribution reflects the tendency of a compound to bind to the tissue components compared with plasma protein binding. In a clinical setting, V_{dist} can be used to determine a loading dose to achieve a steady state concentration. In some embodiments, the volume of distribution of the GDA when co-administered with a delivery agent as described herein can decrease at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%.

### Formulation, Administration, Delivery and Dosing

In some embodiments, GDAs comprise compositions and/or complexes in combination with one or more pharmaceutically acceptable excipients. Pharmaceutical compositions may optionally comprise one or more additional active substances, e.g. therapeutically and/or prophylactically active substances. General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference).

In some embodiments, compositions are administered to humans, human patients or subjects. For the purposes of the present disclosure, the phrase “active ingredient” generally refers to GDAs to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, or at least 80% (w/w) active ingredient. In one embodiment, active ingredients are antibodies directed toward regulatory elements and/or GPCs.

### Formulations

GDAs of the invention can be formulated using one or more excipients to: (1) increase stability; (2) increase cell permeability; (3) permit the sustained or delayed release (e.g., from a formulation of the GDA); and/or (4) alter the biodistribution (e.g., target the GDA to specific tissues or cell types). In addition to traditional excipients such as any and all solvents, dispersion media, diluents, or other liquid vehicles, dispersion or suspension aids, surface active agents, isotonic agents, thickening or emulsifying agents, preservatives, formulations of the present invention can include, without limitation, liposomes, lipid nanoparticles, polymers, lipoplexes, core-shell nanoparticles, peptides, proteins, cells transfected with the GDAs (e.g., for transplantation into a subject) and combinations thereof.

### Excipients

Various excipients for formulating pharmaceutical compositions and techniques for preparing the composition are known in the art (see Remington: The Science and Practice of Pharmacy, 21^{st} Edition, A. R. Gennaro, Lippincott, Williams & Wilkins, Baltimore, Md., 2006; incorporated herein by reference).

The use of a conventional excipient medium is contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEENn®60], polyoxyethylene sorbitan monooleate [TWEEN®80], sorbitan monopalmitate [SPAN®40], sorbitan monostearate [Span®60], sorbitan tristearate [Span® 65], glyceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC® F 68, POLOXAMER 188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN® II, NEOLONE™, KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, Eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

### Formulation Vehicles: Liposomes, Lipoplexes, and Lipid Nanoparticles

GDAs of the invention can be formulated using one or more liposomes, lipoplexes, or lipid nanoparticles. In one embodiment, pharmaceutical compositions of GDA include liposomes. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In one embodiment such formulations may also be constructed or compositions altered such that they passively or actively are directed to different cell types in vivo.

Formulations can also be selectively targeted through expression of different ligands on their surface as exemplified by, but not limited by, folate, transferrin, N-acetylgalactosamine (GalNAc), and antibody targeted approaches.

Liposomes, lipoplexes, or lipid nanoparticles may be used to improve the efficacy of GDA function as these formulations may be able to increase cell transfection by the GDA. The liposomes, lipoplexes, or lipid nanoparticles may also be used to increase the stability of the GDA.

Liposomes that are specifically formulated for antibody cargo are prepared according to techniques known in the art, such as described by Eppstein et al. (Eppstein, D. A. et al., Biological activity of liposome-encapsulated murine interferon gamma is mediated by a cell membrane receptor. Proc Natl Acad Sci USA. 1985 June; 82(11):3688-92); Hwang et al. (Hwang, K. J. et al., Hepatic uptake and degradation of unilamellar sphingomyelin/cholesterol liposomes: a kinetic study. Proc Natl Acad Sci USA. 1980 July; 77(7):4030-4); U.S. Pat. Nos. 4,485,045 and 4,544,545. Production of liposomes with sustained circulation time are also described in U.S. Pat. No. 5,013,556.

Antibody containing liposomes of the present invention may be generated using reverse phase evaporation utilizing lipids such as phosphatidylcholine, cholesterol as well as phosphatidylethanolamine that has been polyethylene glycol-derivatized. Filters with defined pore size are used to extrude liposomes of the desired diameter. In another embodiment, GDAs of the present invention can be conjugated to the external surface of liposomes by disulfide interchange reaction as is described by Martin et al. (Martin, F. J. et al., Irreversible coupling of immunoglobulin fragments to preformed vesicles. An improved method for liposome targeting. J Biol Chem. 1982 Jan. 10; 257(1):286-8).

### Formulation Vehicles: Polymers and Nanoparticles

The GDA of the invention can be formulated using natural and/or synthetic polymers. Non-limiting examples of polymers which may be used for delivery include, but are not limited to DMRI/DOPE, poloxamer, chitosan, cyclodextrin, and poly(lactic-co-glycolic acid) (PLGA) polymers. These may be biodegradable.

The polymer formulation can permit the sustained or delayed release of GDA (e.g., following intramuscular or subcutaneous injection). The altered release profile for the GDA can result in, for example, release of the GDA over an extended period of time. The polymer formulation may also be used to increase the stability of the GDA.

Polymer formulations can also be selectively targeted through expression of different ligands as exemplified by, but not limited by, folate, transferrin, and N-acetylgalactosamine (GalNAc) (Benoit et al., Biomacromolecules. 2011 12:2708-2714; Rozema et al., Proc Natl Acad Sci USA. 2007 104:12982-12887; Davis, Mol Pharm. 2009 6:659-668; Davis, Nature 2010 464:1067-1070; herein incorporated by reference in its entirety).

The GDA of the invention can also be formulated as a nanoparticle using a combination of polymers, lipids, and/or other biodegradable agents, such as, but not limited to, calcium phosphate. Components may be combined in a core-shell, hybrid, and/or layer-by-layer architecture, to allow for fine-tuning of the nanoparticle so delivery of the GDA may be enhanced. For GDA antibodies, systems based on poly(2-(methacryloyloxy)ethyl phosphorylcholine)-block-(2-(diisopropylamino)ethyl methacrylate), (PMPC-PDPA), a pH sensitive diblock copolymer that self-assembles to form nanometer-sized vesicles, also known as polymersomes, at physiological pH may be used. These polymersomes have been shown to successfully deliver relatively high antibody payloads within live cells. (Massignani, et al, Cellular delivery of antibodies: effective targeted subcellular imaging and new therapeutic tool. Nature Proceedings, May, 2010.)

In one embodiment, a PEG-charge-conversional polymer (Pitella et al., Biomaterials. 2011 32:3106-3114) may be used to form a nanoparticle to deliver the GDA of the present invention. The PEG-charge-conversional polymer may improve upon the PEG-polyanion block copolymers by being cleaved into a polycation at acidic pH, thus enhancing endosomal escape.

The use of core-shell nanoparticles has additionally focused on a high-throughput approach to synthesize cationic cross-linked nanogel cores and various shells (Siegwart et al., Proc Natl Acad Sci USA. 2011 108:12996-13001). The complexation, delivery, and internalization of the polymeric nanoparticles can be precisely controlled by altering the chemical composition in both the core and shell components of the nanoparticle.

In one embodiment, matrices of poly(ethylene-co-vinyl acetate), are used to deliver the GDAs of the invention. Such matrices are described in Nature Biotechnology 10, 1446-1449 (1992).

### Antibody Formulations

Antibody GDAs of the invention may be formulated for intravenous administration or extravascular administration (Daugherty, et al., Formulation and delivery issues for monoclonal antibody therapeutics. Adv Drug Deliv Rev. 2006 Aug. 7; 58(5-6):686-706, US patent publication number 2011/0135570, all of which are incorporated herein in their entirety). Extravascular administration routes may include, but are not limited to subcutaneous administration, intraperitoneal administration, intracerebral administration, intraocular administration, intralesional administration, topical administration and intramuscular administration.

Antibody structures may be modified to improve their effectiveness as therapeutics. Improvements may include, but are not limited to improved thermodynamic stability, reduced Fc receptor binding properties and imporved folding efficiency. Modifications may include, but are not limited to amino acid substitutions, glycosylation, palmitoylation and protein conjugation.

Antibody GDAs may be formulated with antioxidants to reduce antibody oxidation. Antibody GDAs may also be formulated with additives to reduce protein aggregation. Such additives may include, but are not limited to albumin, amino acids, sugars, urea, guanidinium chloride, polyalchohols, polymers (such as polyethylene glycol and dextrans), surfactants (including, but not limited to polysorbate 20 and polysorbate 80) or even other antibodies.

Antibody GDAs of the present invention may be formulated to reduce the impact of water on antibody structure and function. Antibody preparartions in such formulations may be may be lyophilized. Formulations subject to lyophilization may include carbohydrates or polyol compounds to protect and stabilize antibody structure. Such compounds include, but are not limited to sucrose, trehalose and mannitol.

Antibody GDAs of the present invention may be formulated with polymers. In one embodiment, polymer formulations may contain hydrophobic polymers. Such polymers may be microspheres formulated with polylactide-co-glycolide through a solid-in-oil-in-water encapsulation method. Microspheres comprising ethylene-vinyl acetate copolymer are also contemplated for antibody delivery and may be used to extend the time course of antibody release at the site of delivery. In another embodiment, polymers may be aqueous gels. Such gels may, for example, comprise carboxymethylcellulose. Aqueous gels may also comprise hyaluronic acid hydrogel. Antibodies may be covalently linked to such gels through a hydrazone linkage that allows for sustained delivery in tissues, including but not limited to the tissues of the central nervous system.

### Formulation Vehicles: Peptides and Proteins

The GDA of the invention can be formulated with peptides and/or proteins. In one embodiment, peptides such as, but not limited to, cell penetrating peptides and proteins and peptides that enable intracellular delivery may be used to deliver pharmaceutical formulations. A non-limiting example of a cell penetrating peptide which may be used with the pharmaceutical formulations of the present invention includes a cell-penetrating peptide sequence attached to polycations that facilitates delivery to the intracellular space, e.g., HIV-derived TAT peptide, penetratins, transportans, or hCT derived cell-penetrating peptides (see, e.g., Caron et al., Mol. Ther. 3(3):310-8 (2001); Langel, Cell-Penetrating Peptides: Processes and Applications (CRC Press, Boca Raton Fla., 2002); El-Andaloussi et al., Curr. Pharm. Des. 11(28):3597-611 (2003); and Deshayes et al., Cell. Mol. Life Sci. 62(16):1839-49 (2005), all of which are incorporated herein by reference). The compositions can also be formulated to include a cell penetrating agent, e.g., liposomes, which enhance delivery of the compositions to the intracellular space. GDAs of the invention may be complexed to peptides and/or proteins such as, but not limited to, peptides and/or proteins from Aileron Therapeutics (Cambridge, Mass.) and Permeon Biologics (Cambridge, Mass.) in order to enable intracellular delivery (Cronican et al., ACS Chem. Biol. 2010 5:747-752; McNaughton et al., Proc. Natl. Acad. Sci. USA 2009 106:6111-6116; Sawyer, Chem Biol Drug Des. 2009 73:3-6; Verdine and Hilinski, Methods Enzymol. 2012; 503:3-33; all of which are herein incorporated by reference in their entirety).

In one embodiment, the cell-penetrating polypeptide may comprise a first domain and a second domain. The first domain may comprise a supercharged polypeptide. The second domain may comprise a protein-binding partner. As used herein, “protein-binding partner” includes, but are not limited to, antibodies and functional fragments thereof, scaffold proteins, or peptides. The cell-penetrating polypeptide may further comprise an intracellular binding partner for the protein-binding partner. The cell-penetrating polypeptide may be capable of being secreted from a cell where the GDA may be introduced.

Formulations of the including peptides or proteins may be used to increase cell transfection by the GDA or alter the biodistribution of the GDA (e.g., by targeting specific tissues or cell types).

### Formulation Vehicles: Cells

Cell-based formulations of the GDA compositions of the invention may be used to ensure cell transfection (e.g., in the cellular carrier) or alter the biodistribution of the compositions (e.g., by targeting the cell carrier to specific tissues or cell types).

### Cell Transfer Methods

A variety of methods are known in the art and suitable for introduction of nucleic acids or proteins into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microproj ectile mediated transfer (nanoparticles), cationic polymer mediated transfer (DEAE-dextran, polyethylenimine, polyethylene glycol (PEG) and the like) or cell fusion.

The technique of sonoporation, or cellular sonication, is the use of sound (e.g., ultrasonic frequencies) for modifying the permeability of the cell plasma membrane. Sonoporation methods are known to those in the art and are used to deliver nucleic acids in vivo (Yoon and Park, Expert Opin Drug Deliv. 2010 7:321-330; Postema and Gilja, Curr Pharm Biotechnol. 2007 8:355-361; Newman and Bettinger, Gene Ther. 2007 14:465-475; all herein incorporated by reference in their entirety). Sonoporation methods are known in the art and are also taught for example as it relates to bacteria in US Patent Publication 20100196983 and as it relates to other cell types in, for example, US Patent Publication 20100009424, each of which are incorporated herein by reference in their entirety.

Electroporation techniques are also well known in the art and are used to deliver nucleic acids in vivo and clinically (Andre et al., Curr Gene Ther. 2010 10:267-280; Chiarella et al., Curr Gene Ther. 2010 10:281-286; Hojman, Curr Gene Ther. 2010 10:128-138; all herein incorporated by reference in their entirety). In one embodiment, GDAs may be delivered by electroporation.

### Administration and Delivery

The compositions of the present invention may be administered by any of the standard methods or routes known in the art.

GDAs of the present invention may be administered by any route which results in a therapeutically effective outcome. These include, but are not limited to enteral, gastroenteral, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection, (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops. In specific embodiments, compositions may be administered in a way which allows them cross the blood-brain barrier, vascular barrier, or other epithelial barrier. Non-limiting routes of administration for the GDAs of the present invention are described below.

### Parenteral and Injectible Administration

Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof. In other embodiments, surfactants are included such as hydroxypropylcellulose.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

### Rectal and Vaginal Administration

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

### Oral Administration

Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

### Topical or Transdermal Administration

As described herein, compositions containing the GDAs of the invention may be formulated for administration topically. The skin may be an ideal target site for delivery as it is readily accessible. Gene expression may be restricted not only to the skin, potentially avoiding nonspecific toxicity, but also to specific layers and cell types within the skin.

The site of cutaneous expression of the delivered compositions will depend on the route of nucleic acid delivery. Three routes are commonly considered to deliver GDAs to the skin: (i) topical application (e.g. for local/regional treatment and/or cosmetic applications); (ii) intradermal injection (e.g. for local/regional treatment and/or cosmetic applications); and (iii) systemic delivery (e.g. for treatment of dermatologic diseases that affect both cutaneous and extracutaneous regions). GDAs can be delivered to the skin by several different approaches known in the art.

In one embodiment, the invention provides for a variety of dressings (e.g., wound dressings) or bandages (e.g., adhesive bandages) for conveniently and/or effectively carrying out methods of the present invention. Typically dressing or bandages may comprise sufficient amounts of pharmaceutical compositions and/or GDAs described herein to allow a user to perform multiple treatments of a subject(s).

In one embodiment, the invention provides for the GDAs compositions to be delivered in more than one injection.

Dosage forms for topical and/or transdermal administration of a composition may include ointments, pastes, creams, lotions, gels, powders, solutions, sprays, inhalants and/or patches. Generally, an active ingredient is admixed under sterile conditions with a pharmaceutically acceptable excipient and/or any needed preservatives and/or buffers as may be required. Additionally, the present invention contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions.

Topically-administrable formulations may, for example, comprise from about 1% to about 10% (w/w) active ingredient, although the concentration of active ingredient may be as high as the solubility limit of the active ingredient in the solvent. Formulations for topical administration may further comprise one or more of the additional ingredients described herein.

### Depot Administration

As described herein, in some embodiments, the composition is formulated in depots for extended release. Generally, a specific organ or tissue (a “target tissue”) is targeted for administration.

In some aspects of the invention, the GDAs are spatially retained within or proximal to a target tissue. Provided are method of providing a composition to a target tissue of a mammalian subject by contacting the target tissue (which contains one or more target cells) with the composition under conditions such that the composition, in particular the GDA component(s) of the composition, is substantially retained in the target tissue, meaning that at least 10, 20, 30, 40, 50, 60, 70, 80, 85, 90, 95, 96, 97, 98, 99, 99.9, 99.99 or greater than 99.99% of the composition is retained in the target tissue. Advantageously, retention is determined by measuring the amount of the GDA present in the composition that enters one or more target cells. For example, at least 1, 5, 10, 20, 30, 40, 50, 60, 70, 80, 85, 90, 95, 96, 97, 98, 99, 99.9, 99.99 or greater than 99.99% of the GDA administered to the subject are present intracellularly at a period of time following administration. For example, intramuscular injection to a mammalian subject is performed using an aqueous composition containing a GDA and a transfection reagent, and retention of the composition is determined by measuring the amount of the GDA present in the muscle cells.

Certain aspects of the invention are directed to methods of providing a composition to a target tissue of a mammalian subject, by contacting the target tissue (containing one or more target cells) with the composition under conditions such that the composition is substantially retained in the target tissue. The composition contains an effective amount of a GDA such that the effect of interest is produced in at least one target cell. The compositions generally contain a cell penetration agent, although “naked” GDAs (such as GDAs without a cell penetration agent or other agent) is also contemplated, and a pharmaceutically acceptable carrier.

In some circumstances, the amount of a growth factor present in cells in a tissue is desirably increased. Preferably, this increase in growth factor is spatially restricted to cells within the target tissue. Thus, provided are methods of increasing the amount of growth factor of interest in a tissue of a mammalian subject. A composition is provided that contains GDAs characterized in that a unit quantity of composition has been determined to produce the level of growth factor of interest in a substantial percentage of cells contained within a predetermined volume of the target tissue.

In some embodiments, the composition includes a plurality of different GDAs, where one or more than one of the GDAs targets a biomolecule of interest. Optionally, the composition also contains a cell penetration agent to assist in the intracellular delivery of the composition. A determination is made of the dose of the composition required to target the biomolecule of interest in a substantial percentage of cells contained within the predetermined volume of the target tissue (generally, without targeting a biomolecule of interest in tissue adjacent to the predetermined volume, or distally to the target tissue). Subsequent to this determination, the determined dose is introduced directly into the tissue of the mammalian subject.

In one embodiment, the invention provides for the GDAs to be delivered in more than one injection or by split dose injections.

### Pulmonary Administration

A pharmaceutical composition may be prepared, packaged, and/or sold in a formulation suitable for pulmonary administration via the buccal cavity. Such a formulation may comprise dry particles which comprise the active ingredient and which have a diameter in the range from about 0.5 nm to about 7 nm or from about 1 nm to about 6 nm. Such compositions are suitably in the form of dry powders for administration using a device comprising a dry powder reservoir to which a stream of propellant may be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (w/w) of the composition, and active ingredient may constitute 0.1% to 20% (w/w) of the composition. A propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

### Intranasal, Nasal and Buccal Administration

Formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

### Ophthalmic or Otic Administration

A pharmaceutical composition may be prepared, packaged, and/or sold in a formulation suitable for ophthalmic or otic administration. Such formulations may, for example, be in the form of eye or ear drops including, for example, a 0.1/1.0% (w/w) solution and/or suspension of the active ingredient in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Subretinal inserts may also be used as form of administration.

### Payload Administration: Detectable Agents and Therapeutic Agents

GDAs described herein can be used in a number of different scenarios in which delivery of a substance (the “payload”) to a biological target is desired, for example delivery of detectable substances for detection of the target, or delivery of a therapeutic or diagnostic agent. Detection methods can include, but are not limited to, both imaging in vitro and in vivo imaging methods, e.g., immunohistochemistry, bioluminescence imaging (BLI), Magnetic Resonance Imaging (MRI), positron emission tomography (PET), electron microscopy, X-ray computed tomography, Raman imaging, optical coherence tomography, absorption imaging, thermal imaging, fluorescence reflectance imaging, fluorescence microscopy, fluorescence molecular tomographic imaging, nuclear magnetic resonance imaging, X-ray imaging, ultrasound imaging, photoacoustic imaging, lab assays, or in any situation where tagging/staining/imaging is required.

GDAs can be designed to include both a linker and a payload in any useful orientation. For example, a linker having two ends is used to attach one end to the payload and the other end to the GDA. The GDAs of the invention can include more than one payload as well as a cleavable linker. In another example, a drug that may be attached to the GDAs via a linker and may be fluorescently labeled can be used to track the drug in vivo, e.g. intracellularly.

Other examples include, but are not limited to, the use of GDAs in reversible drug delivery into cells.

GDAs described herein can be used in intracellular targeting of a payload, e.g., detectable or therapeutic agent, to specific organelle. In addition, the GDAs described herein can be used to deliver therapeutic agents to cells or tissues, e.g., in living animals. For example, the GDAs described herein can be used to deliver chemotherapeutics agents to kill cancer cells. The GDAs attached to the therapeutic agent through a linker can facilitate member permeation allowing the therapeutic agent to travel into a cell to reach an intracellular target.

In some embodiments, the payload may be a therapeutic agent such as a cytotoxin, radioactive ion, chemotherapeutic, or other therapeutic agent. A cytotoxin or cytotoxic agent includes any agent that may be detrimental to cells. Examples include, but are not limited to, taxol, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020 incorporated herein in its entirety), rachelmycin (CC-1065, see U.S. Pat. Nos. 5,475,092, 5,585,499, and 5,846,545, all of which are incorporated herein by reference), and analogs or homologs thereof. Radioactive ions include, but are not limited to iodine (e.g., iodine 125 or iodine 131), strontium 89, phosphorous, palladium, cesium, iridium, phosphate, cobalt, yttrium 90, samarium 153, and praseodymium. Other therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thiotepa chlorambucil, rachelmycin (CC-1065), melphalan, carmustine (BSNU), lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

In some embodiments, the payload may be a detectable agent, such as various organic small molecules, inorganic compounds, nanoparticles, enzymes or enzyme substrates, fluorescent materials, luminescent materials (e.g., luminol), bioluminescent materials (e.g., luciferase, luciferin, and aequorin), chemiluminescent materials, radioactive materials (e.g., ¹⁸F, ⁶⁷Ga, ^{81m}Kr, ⁸²Rb, ¹¹¹In ¹²³I, ¹³³Xe, ²⁰¹Tl, ¹²⁵I, ³⁵S, ¹⁴C, ³H, or ^{99m}Tc (e.g., as pertechnetate (technetate(VII), TcO₄⁻)), and contrast agents (e.g., gold (e.g., gold nanoparticles), gadolinium (e.g., chelated Gd), iron oxides (e.g., superparamagnetic iron oxide (SPIO), monocrystalline iron oxide nanoparticles (MIONs), and ultrasmall superparamagnetic iron oxide (USPIO)), manganese chelates (e.g., Mn-DPDP), barium sulfate, iodinated contrast media (iohexol), microbubbles, or perfluorocarbons). Such optically-detectable labels include for example, without limitation, 4-acetamido-4′-isothiocyanatostilbene-2,2′disulfonic acid; acridine and derivatives (e.g., acridine and acridine isothiocyanate); 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives (e.g., coumarin, 7-amino-4-methylcoumarin (AMC, Coumarin 120), and 7-amino-4-trifluoromethylcoumarin (Coumarin 151)); cyanine dyes; cyanosine; 4′,6-diaminidino-2-phenylindole (DAPI); 5′ 5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]-naphthalene-1-sulfonyl chloride (DNS, dansylchloride); 4-dimethylaminophenylazophenyl-4′-isothiocyanate (DABITC); eosin and derivatives (e.g., eosin and eosin isothiocyanate); erythrosin and derivatives (e.g., erythrosin B and erythrosin isothiocyanate); ethidium; fluorescein and derivatives (e.g., 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, X-rhodamine-5-(and-6)-isothiocyanate (QFITC or XRITC), and fluorescamine); 2-[2-[3-[[1,3-dihydro-1,1-dimethyl-3-(3-sulfopropyl)-2H-benz[e]indol-2-ylidene]ethylidene]-2-[4-(ethoxycarbonyl)-1-piperazinyl]-1-cyclopenten-1-yl]ethenyl]-1,1-dimethyl-3-(3-sulforpropyl)-1H-benz[e]indolium hydroxide, inner salt, compound with n,n-diethylethanamine(1:1) (IR144); 5-chloro-2-[2-[3-[(5-chloro-3-ethyl-2(3H)-benzothiazol-ylidene)ethylidene]-2-(diphenylamino)-1-cyclopenten-1-yl]ethenyl]-3-ethyl benzothiazolium perchlorate (IR140); Malachite Green isothiocyanate; 4-methylumbelliferone orthocresolphthalein; nitrotyrosine; pararosaniline; Phenol Red; B-phycoerythrin; o-phthaldialdehyde; pyrene and derivatives(e.g., pyrene, pyrene butyrate, and succinimidyl 1-pyrene); butyrate quantum dots; Reactive Red 4 (CIBACRON™ Brilliant Red 3B-A); rhodamine and derivatives (e.g., 6-carboxy-X-rhodamine (ROX), 6-carboxyrhodamine (R6G), lissamine rhodamine B sulfonyl chloride rhodarnine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N letramethyl-6-carboxyrhodamine (TAMRA) tetramethyl rhodamine, and tetramethyl rhodamine isothiocyanate (TRITC)); riboflavin; rosolic acid; terbium chelate derivatives; Cyanine-3 (Cy3); Cyanine-5 (Cy5); cyanine-5.5 (Cy5.5), Cyanine-7 (Cy7); IRD 700; IRD 800; Alexa 647; La Jolta Blue; phthalo cyanine; and naphthalo cyanine.

In some embodiments, the detectable agent may be a non-detectable precursor that becomes detectable upon activation (e.g., fluorogenic tetrazine-fluorophore constructs (e.g., tetrazine-BODIPY FL, tetrazine-Oregon Green 488, or tetrazine-BODIPY TMR-X) or enzyme activatable fluorogenic agents (e.g., PROSENSE® (VisEn Medical))). In vitro assays in which the enzyme labeled compositions can be used include, but are not limited to, enzyme linked immunosorbent assays (ELISAs), immunoprecipitation assays, immunofluorescence, enzyme immunoassays (EIA), radioimmunoassays (RIA), and Western blot analysis.

### Combinations

The GDAs may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, diagnostic, or imaging compositions in combination with agents that may improve their bioavailability, reduce and/or modify their metabolism, inhibit their excretion, and/or modify their distribution within the body.

### Dosing and Dosage Forms

The present disclosure encompasses delivery of GDAs for any of therapeutic, pharmaceutical, diagnostic or imaging by any appropriate route taking into consideration likely advances in the sciences of drug delivery. Delivery may be naked or formulated.

### Naked Delivery

GDAs of the present invention may be delivered to cells, tissues, organs or organisms in naked form. As used herein in, the term “naked” refers to GDAs delivered free from agents or modifications which promote transfection or permeability. The naked GDAs may be delivered to the cell, tissue, organ or organism using routes of administration known in the art and described herein. Naked delivery may include formulation in a simple buffer such as saline or PBS.

### Formulated Delivery

GDAs of the present invention may be formulated, using methods described herein. Formulations may contain GDAs which may be modified and/or unmodified. Formulations may further include, but are not limited to, cell penetration agents, pharmaceutically acceptable carriers, delivery agents, bioerodible or biocompatible polymers, solvents, and sustained-release delivery depots. Formulated GDAs may be delivered to cells using routes of administration known in the art and described herein.

Compositions may also be formulated for direct delivery to organs or tissues in any of several ways in the art including, but not limited to, direct soaking or bathing, via a catheter, by gels, powder, ointments, creams, gels, lotions, and/or drops, by using substrates such as fabric or biodegradable materials coated or impregnated with compositions, and the like.

### Dosing

The present invention provides methods comprising administering one or more GDAs in accordance with the invention to a subject in need thereof. Nucleic acids encoding GDAs, proteins or complexes comprising GDAs, or pharmaceutical, imaging, diagnostic, or prophylactic compositions thereof, may be administered to a subject using any amount and any route of administration effective for preventing, treating, diagnosing, or imaging a disease, disorder, and/or condition. The exact amount required will vary from subject to subject, depending on the species, age, and general condition of the subject, the severity of the disease, the particular composition, its mode of administration, its mode of activity, and the like. Compositions in accordance with the invention are typically formulated in dosage unit form for ease of administration and uniformity of dosage. It will be understood, however, that the total daily usage of the compositions of the present invention will be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In certain embodiments, compositions in accordance with the present invention may be administered at dosage levels sufficient to deliver from about 0.0001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

According to the present invention, GDAs may be administered in split-dose regimens. As used herein, a “split dose” is the division of single unit dose or total daily dose into two or more doses, e.g., two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24 hr period. It may be administered as a single unit dose. In one embodiment, the GDA of the present invention are administered to a subject in split doses. The GDA may be formulated in buffer only or in a formulation described herein. A GDA pharmaceutical composition described herein can be formulated into a dosage form described herein, such as a topical, intranasal, intratracheal, or injectable (e.g., intravenous, intraocular, intravitreal, intramuscular, intracardiac, intraperitoneal, subcutaneous). General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference).

### Coatings or Shells

Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

### V. Kits and Devices

### Kits

Any of the compositions described herein may be comprised in a kit. In a non-limiting example, reagents for generating GDAs, including antigen molecules are included in a kit. The kit may further include reagents or instructions for creating or synthesizing the GDA. It may also include one or more buffers. Other kits of the invention may include components for making a GDA protein or nucleic acid array or library and thus, may include, for example, a solid support.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit (labeling reagent and label may be packaged together), the kit also will generally contain a second, third or other additional container into which the additional components may be separately placed. The kits may also comprise a second container means for containing a sterile, pharmaceutically acceptable buffer and/or other diluent. However, various combinations of components may be comprised in a vial. The kits of the present invention also will typically include a means for containing the GDAs, e.g., proteins, nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits of the invention. The dye may then be resuspended in any suitable solvent, such as DMSO.

A kit can include instructions for employing the kit components as well the use of any other reagent not included in the kit. Instructions may include variations that can be implemented.

### Devices

Any of the compositions described herein may be combined with, coated onto or embedded in a device. Devices include, but are not limited to, dental implants, stents, bone replacements, artificial joints, valves, pacemakers or other implantable therapeutic device.

### VI. Definitions

At various places in the present specification, substituents of compounds of the present disclosure are disclosed in groups or in ranges. It is specifically intended that the present disclosure include each and every individual subcombination of the members of such groups and ranges. The following is a non-limiting list of term definitions.

Activity: As used herein, the term “activity” means the condition in which things are happening or being done. Compositions of the invention may have activity and this activity may involve one or more biological events which affect growth factors, receptors, GDAs, GPCs and/or GPC modulatory factors. In some embodiments, the biological event may include cell signaling events associated with growth factor and receptor interactions. In some embodiments, the biological event may include cell signaling events associated with TGF-beta or TGF-beta family member interactions with one or more corresponding receptors.

Administered in combination: As used herein, the term “administered in combination” or “combined administration” means that a subject is simultaneously exposed to two or more agents administered at the same time or within an interval such that the subject is at some point in time simultaneously exposed to both and/or such that there may be an overlap in the effect of each agent on the patient. In some embodiments, at least one dose of one or more agents is administered within about 24 hours, 12 hours, 6 hours, 3 hours, 1 hour, 30 minutes, 15 minutes, 10 minutes, 5 minutes, or 1 minute of at least one dose of one or more other agents. In some embodiments, administration occurs in overlapping dosage regimens. As used herein, the term “dosage regimen” refers to a plurality of doses spaced apart in time. Such doses may occur at regular intervals or may include one or more hiatus in administration. In some embodiments, the administration of individual doses of one or more GDAs, as described herein, are spaced sufficiently closely together such that a combinatorial (e.g., a synergistic) effect is achieved.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Antigens of interest or desired antigens: As used herein, the terms “antigens of interest” or “desired antigens” include those proteins and other biomolecules provided herein that are immunospecifically bound or interact with by the antibodies and fragments, mutants, variants, and alterations thereof described herein. In some embodiments, an antigen of interest may comprise a growth factor, growth factor regulatory element, prodomain, GPC, GPC modulatory factor, ECCM or a region of overlap between them.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Bifunctional: As used herein, the term “bifunctional” refers to any substance, molecule or moiety which is capable of or maintains at least two functions. The functions may affect the same outcome or a different outcome. The structure that produces the function may be the same or different.

Biomolecule: As used herein, the term “biomolecule” is any natural molecule which is amino acid-based, nucleic acid-based, carbohydrate-based or lipid-based, and the like.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, a GDA of the present invention may be considered biologically active if even a portion of the GDA is biologically active or mimics an activity considered biologically relevant.

Biological system: As used herein, the term “biological system” refers to a group of organs, tissues, cells, intracellular components, proteins, nucleic acids, molecules (including, but not limited to biomolecules) that function together to perform a certain biological task within cellular membranes, cellular compartments, cells, tissues, organs, organ systems, multicellular organisms, or any biological entity. In some embodiments, biological systems are cell signaling pathways comprising intracellular and/or extracellular cell signaling biomolecules. In some embodiments, biological systems comprise growth factor signaling events within the ECCM and/or cellular niches.

Compound: As used herein, the term “compound,” refers to a distinct chemical entity. In some embodiments, a particular compound may exist in one or more isomeric or isotopic forms (including, but not limited to stereoisomers, geometric isomers and isotopes). In some embodiments, a compound is provided or utilized in only a single such form. In some embodiments, a compound is provided or utilized as a mixture of two or more such forms (including, but not limited to a racemic mixture of stereoisomers). Those of skill in the art appreciate that some compounds exist in different such forms, show different properties and/or activities (including, but not limited to biological activities). In such cases it is within the ordinary skill of those in the art to select or avoid particular forms of the compound for use in accordance with the present invention. For example, compounds that contain asymmetrically substituted carbon atoms can be isolated in optically active or racemic forms. Methods on how to prepare optically active forms from optically active starting materials are known in the art, such as by resolution of racemic mixtures or by stereoselective synthesis.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved among more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an oligonucleotide or polypeptide or may apply to a portion, region or feature thereof.

In one embodiment, conserved sequences are not contiguous. Those skilled in the art are able to appricate how to achieve alignment when gaps in contiguous alignment are present between sequences, and to align corresponding residues not withstanding insertions or deletions present.

Cyclic or Cyclized: As used herein, the term “cyclic” refers to the presence of a continuous loop. Cyclic molecules need not be circular, only joined to form an unbroken chain of subunits. Cyclic molecules such as certain GDAs as described herein may be single units or multimers or comprise one or more components of a complex or higher order structure.

Cytostatic: As used herein, the term “cytostatic” is used to refer an agent that inhibits, reduces or suppresses the growth, division, or multiplication of a cell (e.g., a mammalian cell (e.g., a human cell)), bacterium, virus, fungus, protozoan, parasite, prion, or a combination thereof.

Cytotoxic: As used herein, the term “cytotoxic” is used to refer to an agent that kills or causes injurious, toxic, or deadly effects on a cell (e.g., a mammalian cell (e.g., a human cell)), bacterium, virus, fungus, protozoan, parasite, prion, or a combination thereof.

Delivery: As used herein, “delivery” refers to the act or manner of delivering a compound, substance, entity, moiety, cargo or payload.

Delivery Agent: As used herein, “delivery agent” refers to any substance which facilitates, at least in part, the in vivo delivery of an agent (including, but not limited to a GDA) to targeted cells.

Destabilized: As used herein, the term “destable,” “destabilize,” or “destabilizing region” means a region or molecule that is less stable than a starting, reference, wild-type or native form of the same region or molecule.

Detectable label: As used herein, “detectable label” refers to one or more markers, signals, or moieties which are attached, incorporated or associated with another entity, which markers, signals or moieties are readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the entity with which they are attached, incorporated or associated. For example, when attached, incorporated in or associated with a peptide or protein, they may be within the amino acids, the peptides, or proteins, or located at the N- or C-termini.

Distal: As used herein, the term “distal” means situated away from the center or away from a point or region of interest.

Engineered: As used herein, embodiments of the invention are “engineered” when they are designed to have a feature or property, whether structural or chemical, that varies from a starting point, wild type or native molecule. Thus, engineered agents or entities are those whose design and/or production include an act of the hand of man.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; (4) folding of a polypeptide or protein; and (5) post-translational modification of a polypeptide or protein.

Feature: As used herein, a “feature” refers to a characteristic, a property, or a distinctive element.

Formulation: As used herein, a “formulation” includes at least a GDA and a delivery agent.

Fragment: A “fragment,” as used herein, refers to a portion. For example, fragments of proteins may comprise polypeptides obtained by digesting full-length protein isolated from cultured cells. In some embodiments, a fragment of a protein includes at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250 or more amino acids. In some embodiments, fragments of an antibody include portions of an antibody subjected to enzymatic digestion or synthesized as such.

Functional: As used herein, a “functional” biological molecule is a biological entity with a structure and in a form in which it exhibits a property and/or activity by which it is characterized.

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the invention, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is typically determined by the ability to encode a stretch of at least 4-5 uniquely specified amino acids. In accordance with the invention, two protein sequences are considered to be homologous if the proteins are at least about 50%, 60%, 70%, 80%, or 90% identical for at least one stretch of at least about 20 amino acids. In many embodiments, homologous protein may show a large overall degree of homology and a high degree of homology over at least one short stretch of at least 3, 4, 5, 6, 7, 8, 9, 10, 11, 12, 13, 14, 15, 16, 17, 18, 19, 20, 25, 30, 35, 40, 45, 50 or more amino acids. In many embodiments, homologous proteins share one or more characteristic sequence elements. As used herein, the term “characteristic sequence element” refers to a motif present in related proteins. In some embodiments, the presence of such motifs correlates with a particular activity (such as biological activity).

Identity: As used herein, the term “identity” refers to the overall relatedness between polymeric molecules, e.g., between oligonucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined, for example using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically a reduction in the level of an mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” is synonymous with “separated”, but carries with it the inference separation was carried out by the hand of man. In one embodiment, an isolated substance or entity is one that has been separated from at least some of the components with which it was previously associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art. In some embodiments, isolation of a substance or entity includes disruption of chemical associations and/or bonds. In some embodiments, isolation includes only the separation from components with which the isolated substance or entity was previously combined and does not include such disruption.

Linker: As used herein, a linker refers to a moiety that connects two or more domains, moieties or entities. In one embodiment, a linker may comprise 10 or more atoms. In a further embodiment, a linker may comprise a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. In some embodiments, a linker may comprise one or more nucleic acids comprising one or more nucleotides. In some embodiments, the linker may comprise an amino acid, peptide, polypeptide or protein. In some embodiments, a moiety bound by a linker may include, but is not limited to an atom, a chemical group, a nucleoside, a nucleotide, a nucleobase, a sugar, a nucleic acid, an amino acid, a peptide, a polypeptide, a protein, a protein complex, a payload (e.g., a therapeutic agent). or a marker (including, but not limited to a chemical, fluorescent, radioactive or bioluminescent marker). The linker can be used for any useful purpose, such as to form multimers or conjugates, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers, Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bonds include an amido bond which may be cleaved for example by the use of tris(2-carboxyethyl)phosphine (TCEP), or other reducing agents, and/or photolysis, as well as an ester bond which may be cleaved for example by acidic or basic hydrolysis.

Modified: As used herein, the term “modified” refers to a changed state or structure of a molecule or entity as compared with a parent or reference molecule or entity. Molecules may be modified in many ways including chemically, structurally, and functionally. In some embodiments, GDAs of the present invention are modified by the introduction of non-natural amino acids.

Naturally occurring: As used herein, “naturally occurring” means existing in nature without artificial aid, or involvement of the hand of man.

Non-human vertebrate: As used herein, a “non human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Off-target: As used herein, “off target” refers to any unintended effect on any one or more target, gene, or cellular transcript.

Operably linked: As used herein, the phrase “operably linked” refers to a functional connection between two or more molecules, constructs, transcripts, entities, moieties or the like.

Paratope: As used herein, a “paratope” refers to the antigen-binding site of an antibody.

Patient: As used herein, “patient” refers to a subject who may seek or be in need of treatment, requires treatment, is receiving treatment, will receive treatment, or a subject who is under care by a trained (e.g., licensed) professional for a particular disease or condition.

Peptide: As used herein, “peptide” is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than active agents (e.g., as described herein) present in a pharmaceutical composition and having the properties of being substantially nontoxic and non-inflammatory in a patient. In some embodiments, a pharmaceutically acceptable excipient is a vehicle capable of suspending or dissolving the active agent. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: Pharmaceutically acceptable salts of the compounds described herein are forms of the disclosed compounds wherein the acid or base moiety is in its salt form (e.g., as generated by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Pharmaceutically acceptable salts include the conventional non-toxic salts, for example, from non-toxic inorganic or organic acids. In some embodiments a pharmaceutically acceptable salt is prepared from a parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety. Pharmaceutically acceptable solvate: The term “pharmaceutically acceptable solvate,” as used herein, refers to a crystalline form of a compound wherein molecules of a suitable solvent are incorporated in the crystal lattice. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.” In some embodiments, the solvent incorporated into a solvate is of a type or at a level that is physiologically tolerable to an organism to which the solvate is administered (e.g., in a unit dosage form of a pharmaceutical composition).

Pharmacokinetic: As used herein, “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prodrug: The present disclosure also includes prodrugs of the compounds described herein. As used herein, “prodrugs” refer to any substance, molecule or entity which is in a form predicate for that substance, molecule or entity to act as a therapeutic upon chemical or physical alteration. Prodrugs may by covalently bonded or sequestered in some way and which release or are converted into the active drug moiety prior to, upon or after administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

Proliferate: As used herein, the term “proliferate” means to grow, expand, replicate or increase or cause to grow, expand, replicate or increase. “Proliferative” means having the ability to proliferate. “Anti-proliferative” means having properties counter to or in opposition to proliferative properties.

Protein of interest: As used herein, the terms “proteins of interest” or “desired proteins” include those provided herein and fragments, mutants, variants, and alterations thereof.

Proximal: As used herein, the term “proximal” means situated nearer to the center or to a point or region of interest.

Purified: As used herein, “purify,” means to make substantially pure or clear from unwanted components, material defilement, admixture or imperfection. “Purified” refers to the state of being pure. “Purification” refers to the process of making pure.

Sample: As used herein, the term “sample” refers to an aliquot or portion taken from a source and/or provided for analysis or processing. In some embodiments, a sample is from a biological source such as a tissue, cell or component part (e.g. a body fluid, including but not limited to blood, mucus, lymphatic fluid, synovial fluid, cerebrospinal fluid, saliva, amniotic fluid, amniotic cord blood, urine, vaginal fluid and semen). In some embodiments, a sample may be or comprise a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. In some embodiments, a sample is a or comprises a medium, such as a nutrient broth or gel, which may contain cellular components, such as proteins or nucleic acid molecule. In some embodiments, a “primary” sample is an aliquot of the source. In some embodiments, a primary sample is subjected to one or more processing (e.g., separation, purification, etc.) steps to prepare a sample for analysis or other use.

Signal Sequences: As used herein, the phrase “signal sequences” refers to a sequence which can direct the transport or localization of a protein.

Single unit dose: As used herein, a “single unit dose” is a dose of any therapeutic administered in one dose/at one time/single route/single point of contact, i.e., single administration event. In some embodiments, a single unit dose is provided as a discrete dosage form (e.g., a tablet, capsule, patch, loaded syringe, vial, etc.).

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Split dose: As used herein, a “split dose” is the division of single unit dose or total daily dose into two or more doses.

Stable: As used herein “stable” refers to a compound or entity that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable. In some embodiments, stability is measured relative to an absolute value. In some embodiments, stability is measured relative to a reference compound or entity.

Subject: As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term typically means within about 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Synthetic: The term “synthetic” means produced, prepared, and/or manufactured by the hand of man. Synthesis of polynucleotides or polypeptides or other molecules of the present invention may be chemical or enzymatic.

Targeted Cells: As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24 hr period. It may be administered as a single unit dose.

Transcription factor: As used herein, the term “transcription factor” refers to a DNA-binding protein that regulates transcription of DNA into RNA, for example, by activation or repression of transcription. Some transcription factors effect regulation of transcription alone, while others act in concert with other proteins. Some transcription factor can both activate and repress transcription under certain conditions. In general, transcription factors bind a specific target sequence or sequences highly similar to a specific consensus sequence in a regulatory region of a target gene. Transcription factors may regulate transcription of a target gene alone or in a complex with other molecules.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild type or native form of a biomolecule or entity. Molecules or entities may undergo a series of modifications whereby each modified product may serve as the “unmodified” starting molecule or entity for a subsequent modification.

### VII. Equivalents and Scope

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

### Example 1. Identification and Selection of Antigens

In order to identify and select the antigens used in the preparation of the GDA antibodies of the present invention, investigations into the structure-activity relationship of the TGF-beta family was undertaken. The methods used were those described in Shi, M. et al., Latent TGF-β structure and activation. Nature. 2011 Jun. 15; 474(7351):343-9.

### Structural Analysis of TGF-Beta

The structure of pro-TGF-beta1 has a ring-like shape. Two prodomain arm domains connect at the elbows to crossed ‘forearms’ formed by the two growth-factor monomers and by prodomain ‘straitjacket’ elements that surround each growth-factor monomer. The centre of the ring contains solvent. The arms come together at the neck, where they are disulphide-linked in a bowtie, and RGD motifs locate to each shoulder. On the opposite side of the ring where the straitjacketed forearms cross, is the site where LTBP links to straitjacket residue Cys 4 of the alpha1 helix.

### Structural Alignment Among TGF-Beta Family Members

Given the structural insights obtained from crystal structure analysis of TGF-beta1 and the identification of target sites for antibody development, it was of interest to conduct sequence alignments with the other members of the TGF-beta family to identify corresponding targets. The TGF-beta family consists of 33 members. Although growth-factor domains are highly conserved, prodomains vary in length from 169 to 433 residues, and are variously described as unrelated in sequence or low in homology. However, alignment shows that all prodomains have a similar fold. Deeply buried hydrophobic residues in core secondary-structure elements of the arm domain, that is, the α2 helix and β-strands 1-3, 6, 7 and 10, are conserved in all members.

Most family members also contain clear sequence signatures for the amphipathic C-terminal portion of the α1 helix that inserts intimately between the two growth-factor monomers. A similar insertion in inhibin-α and inhibin-βA has been demonstrated by mapping disruptive mutations to the equivalents of Ile 24 and Leu 28 in TGF-beta. Many family members also contain proline-rich latency lasso loops with lengths that are compatible with encirclement of the growth-factor β-finger. Thus, a prodomain structure similar to that of proTGF-beta, including a portion of the straitjacket, is widespread in the TGF-beta family. However, the low sequence identity and many insertions and deletions indicate substantial specializations.

Differences in prodomain dimerization among family members are indicated by variations in cysteine positions. The bowtie (β-strands 8 and 9) and its disulphides are specializations. Inhibin-α and -β sub-units have cysteines in similar positions, whereas other family members either have cysteine residues in the β7 strand or lack cysteines altogether in this region.

The interface between the two arm domains in the β4 and β5 strands is modest in size and lacks hydrophobic and conserved residues. GDF1 and GDF15 specifically lack the β4 and β5 strands, which are adjacent in sequence and structure, on the edge of a β-sheet. Therefore, arm-domain dimerization seems to be variable or absent in some family members.

The close relatives of TGF-beta, myostatin and GDF11, which are also latent, show conservation of the fastener residues Lys 27 and Tyr 75. Myostatin regulates muscle mass and is stored in the extracellular matrix, bound to LTBP3. Release of myostatin and GDF11 from latency requires cleavage of the prodomain between Arg 75 and Asp 76 by BMP1/tolloid metalloproteinases. This cleavage is between the α2 helix and the fastener. Thus at least two different methods of unfastening the straitjacket, force and proteolysis, can release family members from latency.

An increasingly large number of TGF-beta family members are recognized to remain associated with their prodomains after secretion, including BMP4, BMP7, BMP10, GDF2, GDF5 and GDF8. Furthermore, many of these prodomains bind with high affinity to fibrillin-1 and fibrillin-2. Targeting by the prodomain to the extra-cellular matrix may be of wide importance in regulating bioactivity in the TGF-beta family. Moreover, binding to LTBPs or fibrillins seems to strengthen the prodomain-growth-factor complex. Thus, although only a limited number of TGF-beta family members are latent as prodomain-growth-factor complexes, the concept of latency may extend to other members when their physiologically relevant complexes with LTBPs and fibrillins are considered.

The signalling range of BMP4 in vivo is increased by extracellular cleavage of the prodomain by furin-like proteases at a second site upstream of the prodomain-growth-factor cleavage site. Notably, the second site is in the disordered loop bearing the arginine of RGD in TGF-beta1. Loss of the central β10 strand between the two cleavage sites results in loss of binding of the BMP4 prodomain to its growth factor.

The prodomain of Nodal, which binds to Cripto, targets Nodal for cleavage by proteases secreted by neighbouring cells. AMH is secreted largely uncleaved and association with the prodomain greatly potentiates its activity in vivo. Lefty protein, which is involved in establishing bilateral asymmetry, is not cleaved between the arm and growth-factor domains, and is cleaved instead between the α2 helix and the fastener. Notably, release of the straitjacket should be sufficient to enable access of type II receptors to growth-factor domains.

### Example 2. Generation of Antibodies for Therapeutic Treatments

### Antibody GDAs Produced by Standard Monoclonal Antibody Generation

GDAs of the current invention can be antibodies that specifically target TGF-beta family members, their GPCs or other targets. In one embodiment, such antibodies are generated in knockout mice, lacking the gene that encodes for the desired target antigen. Such mice would not be tolerized to the target antigen and therefore would generate antibodies against it that could cross react with human and mouse forms of the antigen. For the production of monoclonal antibodies, host mice are immunized with the target peptide to elicit lymphocytes that specifically bind that peptide. Lymphocytes are collected and fused with an immortalized cell line. The resulting hybridoma cells are cultured in a suitable culture medium with a selection agent to support the growth of only the fused cells.

Desired hybridoma cell lines are then identified through binding specificity analysis of the secreted antibodies for the target peptide and clones of these cells are subcloned through limiting dilution procedures and grown by standard methods. Antibodies produced by these cells are isolated and purified from the culture medium by standard immunoglobulin purification procedures

### Antibody GDAs Produced Recombinantly

Recombinant antibody GDAs are produced using the hybridoma cells produced above. Heavy and light chain variable region cDNA sequences of the antibody GDA are determined using standard biochemical techniques. Total RNA are extracted from antibody GDA-producing hybridoma cells and converted to cDNA by reverse transcriptase (RT) polymerase chain reaction (PCR). PCR amplification will be carried out on the resulting cDNA using primers specific for amplification of the heavy and light chain sequences. PCR products are then be subcloned into plasmids for sequence analysis. Once sequenced, the antibody GDA coding sequence are placed into an expression vector. For humanization of the antibody produced, coding sequences for human heavy and light chain constant domains are used to substitute for the homologous murine sequences. The resulting construct are transfected into mammalian cells capable of large scale translation of the antibody GDA.

### GDAs Produced by Using Antibody Fragment Display Library Screening Techniques

GDAs of the present invention may be produced using high throughput methods of discovery. In one embodiment, GDAs comprising synthetic antibodies are designed by screening target antigens using a phage display library. The phage display libraries are composed of millions to billions of phage particles, each expressing a unique Fab antibody fragment on their viral coat. The cDNA encoding each Fab contains the same sequence with the exception of a unique sequence encoding the variable loops of the complementarity determining regions (CDRs). The V_{H} chains of the CDR are expressed as a fusion protein, linked to the N-terminus of the viral pIII coat protein. The V_{L} chain is expressed separately and assembles with the V_{H} chain in the periplasm prior to incorporation of the complex into the viral coat. Target antigens are incubated, in vitro, with members of the phage display library and bound phage particles are precipitated. The cDNA encoding the CDRs of the bound Fab subunits is sequenced from the bound phage. These sequences can be directly incorporated into antibody sequences for recombinant antibody production, or be mutated and utilized for further optimization through in vitro affinity maturation.

### GDAs Produced Using Affinity Maturation Techniques

Fabs capable of binding target antigens are identified using the libraries described above and high affinity mutants are derived from these through the process of affinity maturation. Affinity maturation technology is used to identify sequences encoding CDRs that have the highest affinity for the target antigen. Using this technology, the CDR sequences isolated using the phage display library selection process described above are mutated randomly as a whole or at specific residues to create a millions to billions of variants. These variants are expressed in Fab antibody fragment fusion proteins in a phage display library and screened for their ability to bind the target antigen. Several rounds of selection, mutation and expression are carried out to identify antibody fragment sequences with the highest affinity for the target antigen. These sequences can be directly incorporated into antibody sequences for recombinant antibody production and incorporation into GDAs.

### Example 3. In Vivo Testing of GDAs

### Determination of the Efficacy of GDAs in the Treatment of Lung Fibrosis

GDAs of the present invention are utilized in combination with mouse models of disease to assess their efficacy in the treatment of those diseases. In one such case, GDAs produced to treat lung fibrosis are used to treat mice subjected to bleomycin induced lung injury. Injured as well as sham injured mice are given weekly intraperitoneal injections of a GDA directed against TGF-beta. After 30 days, postmortem lung tissue is collected and assayed for levels of hydroxyproline as an indicator of fibrotic activity. Levels of hydroxyproline are then correlated with GDA dose to determine efficacy.

### Determination of the Ability of GDAs to Alter Bone Density

GDAs of the present invention are administered to C57Bl/6 mice daily by injection. Bone mineral density is then analyzed using a densitometer as described by others (Mohammad, K. S. et al., Pharmacologic inhibition of the TGF-beta type I receptor kinase has anabolic and anti-catabolic effects on bone. PLoS One. 2009; 4(4):e5275) and changes in bone mineral density are assessed as a percentage change in the area scanned.

### Use of GDAs to Treat Human Disease

GDAs of the present invention may be used in the treatment of human disease. One such disease is Camurati-Engelmann disease (CED). Patients suffering from CED experience symptoms related to overactive TGF-beta signaling. GDAs designed to treat these patients are designed to stabilize the TGF-beta GPC to decrease TGF-beta signaling. Such GDAs may be directed against GPC regions containing a Y52H mutation in patients with CED where the GDA is specifically designed to stabilize the association between the α2-helix and the TGF-beta fingers. GDAs may also be designed to target a pH-regulated salt bridge between Glu 140 and His 193 of the GPC that is disrupted in CED patients due to H193D and E140K mutations. Such a GDA would be designed to stabilize that region and reduce TGF-beta release from the GPC.

### Example 4. Furin Cleavage Assay

The present invention includes an assay developed for assaying furin-dependent cleavage of GPCs. This assay is carried out using polyacrylamide gel electrophoresis (PAGE) technology. The assay is useful in determining the level of GPC processing. In the case of TGF-beta, the GPC is synthesized as a precursor polypeptide. Upon dimerization, furin-dependent cleavage occurs converting the dimer to a tightly associated complex of four polypeptides that include a prodomain dimer and growth factor dimer. Samples to be assayed are solubilized and separated by PAGE under reducing or non-reducing conditions (to allow the complexes to remain disulfide-linked). GPC that has not undergone furin-dependent cleavage migrates more slowly than the two cleaved components. Resulting bands of protein can be visualized using standard antibody blotting techniques or through total protein staining and their position on the gel can be correlated with the migration of protein standards to determine the GPC fraction contained in each band. Band density may be determined through densitometric analysis and values can be used to determine the level of GPC processing or overall furin cleavage activity.

### Example 5. TGF-Beta Reporter Assay

Transformed mink lung TGF-beta reporter cell (TMLC) lines are cultured in each well of a white, opaque assay plate. TMLC cells comprise a luciferase reporter plasmid that further comprises a luciferase gene controlled by the plasminogen activator inhibitor-1 (PAI-1) gene promoter (Abe, M. et al., An assay for transforming growth factor-beta using cells transfected with a plasminogen activator inhibitor-1 promoter-luciferase construct. Anal Biochem. 1994 Feb. 1; 216(2):276-84). The PAI-1 gene promoter is responsive to TGF-beta-induced cell signaling, leading the TMLC cells to produce luciferase in response to TGF-beta ligand.

Biological samples to be tested for TGF-beta activity are obtained. In some embodiments, samples comprise tissue homogenates, bodily fluids (including, but not limited to blood, urine and spinal fluid), cell culture medium, buffer and the like. In some embodiments, samples are obtained from patients treated with or without GDAs. In some embodiments, samples are obtained from cells and/or tissues grown or maintained in culture with or without exposure to GDAs or commercially obtained TGF-beta. In some embodiments, samples are obtained from animals treated with or without GDAs.

Samples including various amounts of diluent are cultured in TMLC culture plates for a period of at least 4 hours. Cell lysates are collected and analyzed for luciferase activity using a Luciferase Assay System (Promega, Madison, Wis.) and Synergy 2 Multi-Mode Microplate Reader (BioTek, Winooski, Vt.) according to the methods described by Wang, et al. (Wang, R. et al., GARP regulates the bioavailability and activation of TGFβ. Mol Biol Cell. 2012 March; 23(6):1129-39). Data are presented as the mean of triplicate samples tested (+/−SEM).

1.-39. (canceled)

40. A method for selecting an antibody, or an antigen-binding portion thereof, that binds a precursor of a growth factor, thereby modulating a cell signaling event associated with the function of the growth factor, wherein the growth factor is a member of the TGFβ superfamily, the method comprising the steps of:
(a) providing an antigen comprising a growth factor pro-protein dimer or a fragment thereof;
(b) selecting a pool of antibodies or antigen-binding portions thereof for the ability to bind the antigen; and
(c) selecting a pool of antibodies, or antigen-binding portions thereof, for the ability to:
(i) inhibit the release of the growth factor from a growth factor prodomain complex (GPC); or
(ii) increase the level of free growth factor in a cell niche.

41. The method of claim 40, wherein the antibody, or the antigen-binding portion thereof, binds an epitope formed by the combination of a region or portion of any of the growth factor, a prodomain of the growth factor, and an extracellular and cellular matrix (ECCM) component selected from latent TGFβ-binding protein (LTBP), fibrillin, elastin and collagen.

42. The method of claim 40, wherein the growth factor is a TGFβ.

43. The method of claim 42, wherein the TGFβ is TGFβ1.

44. The method of claim 42, wherein the antibody, or the antigen-binding portion thereof, inhibits the release of TGFβ from a GPC associated with a latent TGFβ-binding protein (LTBP) but not those associated with glycoprotein-A repetitions predominant protein (GARP).

45. The method of claim 44, wherein the GPC comprises i) a proTGFβ and ii) an LTBP, or a fragment thereof.

46. The method of claim 40, wherein the antibody, or the antigen-binding portion thereof, binds an epitope comprising a region of a prodomain of the growth factor.

47. The method of claim 40, wherein the TGFβ superfamily member is myostatin.

48. The method of claim 40, wherein the antibody, or the antigen-binding portion thereof, is human or humanized.

49. The method of claim 40, wherein the antibody, or the antigen-binding portion thereof, is of class IgA, IgD, IgE, IgG, or IgM.

50. The method of claim 40, wherein the antibody, or the antigen-binding portion thereof, is a monoclonal antibody.

51. The method of claim 40, further comprising a negative selection step, wherein antibodies, or antigen-binding portions thereof, that bind a free, mature form of the growth factor are removed from the pool.

52. The method of claim 40, wherein the selecting step (b) comprises screening a library.

53. The method of claim 52, wherein the library is selected from the group consisting of: an antibody display library, an antibody fragment display library or an Fab fragment display library.

54. The method of claim 53, wherein the display library is expressed in a host selected from the group consisting of yeast, bacteriophage, bacteria and retroviruses.
